SEQUENCE LISTING (84 sequences)

SEQ ID NO: 1 - DNA, 1183 bp, synthetic construct
  source 1..1183: mol_type = other DNA
ctgtggatggagggagggaaggagggggggagtgagtgagtcgagagaacgacgactggggcaaagagaggatgacgccactgcaacaaaagacagggaagaatagaggctgttggaaaacaggaacacagtgaagaatagaggctgcagggtaacaggcacatttatcgcagagaggcgggcggcaggggcgatacgtaaagagacccctaaacttctccgcgtcatccgttcacgctacctccttccctccctttactcccattgatcaccaaggcagcgcccgcagccaccagcagccaccgtcctcagtgtcacagctagacgcgtccccatcaggcagcagaccaccagaatatgcgctttcgcttcccatcatatccctccctcctccccttctattcctcatcatgcatcatttctttctccttgctgcatgcccctcttcttaccttggctaagatctaccggaggaagaagtcttcaacgatagataaatgcctgtatcgtgatggttgtttgaatggtggaagtgagccaggtattgagaacgaggggcgcaagggtgcgtggaagaaggcagggagaggcgaggcgaaggagaagggcatgtggtgggaacgaagcctccacaccacagccacccatgcttttctctgttcgagttcccgggttctactctcacacactacacgtgcacagaaaacattcacgcacaacagagatacagagggatccgtcgagatggtctacacgtcctaatgtcccctgctttcgaagcccagagccatcgctttctgtttttatccattcgcaacacatcctgttctcgcgacctctcctcttcctggccaccatcgcttctccgtgccatgccctgtacatccttccctccctcgttgtctaccacactaatcatgcgctctgcagcagacactcggctactatttgtgtcgtacggattgaggggagggaggggaagggaggcaagaagtaaatcggtggtcaacaacagacgcggtctccccaccctccctcattcctttcccctcctataatgagcgaagaaagccatggacaccgagtcacccactctcctaatccaccaccaccagggcactaacggactcacgctcaccaaactcggtaacaatatccactgcacacagatacacacgcaacc

SEQ ID NO: 2 - DNA, 1180 bp, synthetic construct
  source 1..1180: mol_type = other DNA
tctgtggatggagggagggaaggagggggggagtgagtgagtcgagagaacgacgactggggcaaagagaggatgacgccactgcaacaaaagacagggaagaatagaggctgttggaaaacaggaacacagtgaagaatagaggctgcagggtaacaggcacatttatcgcagagaggcgggcggcaggggcgatacgtaaagagacccctaaacttctccgcgtcatccgttcacgctacctccttccctccctttactcccattgatcaccaaggcagcgcccgcagccaccagcagccaccgtcctcagtgtcacagctagactcccatcaggcagcagaccaccagaatatgcgctttcgcttcccatcatatccctccctcctccccttctattcctcatcatgcatcatttctttctccttgctgcatgcccctcttcttaccttggctaagatctaccggaggaagaagtcttcaacgatagataaatgcctgtatcgtgatggttgtttgaatggtggaagtgagccaggtattgagaacgaggggcgcaagggtgcgtggaagaaggcagggagaggcgaggcgaaggagaagggcatgtggtgggaacgaagcctccacaccacagccacccatgcttttctctgttcgagttcccgggttctactctcacacactacacgtgcacagaaaacattcacgcacaacagagatacagagggatccgtcgagatggtctacacgtcctaatgtcccctgctttcgaagcccagagccatcgctttctgtttttatccattcgcaacacatcctgttctcacgacctctcctcttcctggccaccatcgcttctccgtgccatgccctgtacatccttccctccctcgttgtctaccacactaatcatgcgctctgcagcagacactcggctactatttgtgtcgtacggattgaggggagggaggggaagggaggcaagaagtaaatcggtggtcaacaacagacgcggtctccccaccctccctcattcctttcccctcctataatgagcgaagaaagccatggacaccgagtcacccactctcctaatccaccaccaccagggcactaacggactcacgctcaccaaactcggtaacaatatccactgcacacagatacacacgcaacc

SEQ ID NO: 3 - protein, 19 aa, unidentified
  source 1..19: note = viral protein
GEGRGSLLTCGDVEENPGP

SEQ ID NO: 4 - protein, 24 aa, unidentified
  source 1..24: note = viral protein
APVKQTLNFDLLKLAGDVESNPGP

SEQ ID NO: 5 - protein, 19 aa, unidentified
  source 1..19: note = viral protein
ATNFSLLKQAGDVEENPGP

SEQ ID NO: 6 - protein, 60 aa, synthetic construct
APVTELLYRMKRAETYCPRPLLAIHPTEARHKQKIVAPVKQTLNFDLLKLAGDVESNPGP

SEQ ID NO: 7 - protein, 29 aa, synthetic construct
MTLLSFQGPGATNFSLLKQAGDVEENPGP

SEQ ID NO: 8 - protein, 60 aa, synthetic construct
FCPRPGIHDPAAFSKSHPENSDGRSERLSTAMTTLSFQGPGATNFSLLKQAGDVEENPGP

SEQ ID NO: 9 - protein, 45 aa, synthetic construct
SHPENSDGRSERLSTAMTTLSFQGPGATNFSLLKQAGDVEENPGP

SEQ ID NO: 10 - protein, 29 aa, synthetic construct
MTTLSFQGPGATNFSLLKQAGDVEENPGP

SEQ ID NO: 11 - DNA, 102 bp, synthetic construct
  source 1..102: mol_type = other DNA
gggtcccgggttctcctccacgtcaccggcctgcttcagcaaggagaagttcgtcgctcccggtccctggaaggacagggtcgtcatgccgcttcccgtacg

SEQ ID NO: 12 - DNA, 84 bp, synthetic construct
  source 1..84: mol_type = other DNA
acgaccctgtccttccaggggccgggggcgacgaacttctccttgctgaagcaggccggggacgtggaggagaacccggggccc

SEQ ID NO: 13 - DNA, 84 bp, synthetic construct
  source 1..84: mol_type = other DNA
accactctctcatttcaaggacccggagctacaaacttctctttactaaagcaagcaggggatgttgaagaaaaccccgggccc

SEQ ID NO: 14 - protein, 8 aa, synthetic construct
DXEXNPGP

SEQ ID NO: 15 - protein, 7 aa, synthetic construct
PKKKRKV

SEQ ID NO: 16 - DNA, 106 bp, unidentified
  source 1..106: mol_type = genomic DNA; note = adeno-associated virus
ctgcgcgctcgctcgctcactgaggccgcccgggcaaagcccgggcgtcgggcgacctttggtcgcccggcctcagtgagcgagcgagcgcgcagagagggagtgg

SEQ ID NO: 17 - DNA, 143 bp, unidentified
  source 1..143: mol_type = genomic DNA; note = adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 18 - DNA, 410 bp, synthetic construct
  source 1..410: mol_type = other DNA
tgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataa

SEQ ID NO: 19 - DNA, 176 bp, synthetic construct
  source 1..176: mol_type = other DNA
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 20 - DNA, 161 bp, synthetic construct
  source 1..161: mol_type = other DNA
gtgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggtt

SEQ ID NO: 21 - DNA, 270 bp, synthetic construct
  source 1..270: mol_type = other DNA
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 22 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 23 - DNA, 280 bp, synthetic construct
  source 1..280: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 24 - DNA, 72 bp, synthetic construct
  source 1..72: mol_type = other DNA
gggggaggctgctggtgaatattaaccaaggtcaccccagttatcggaggagcaaacaggggctaagtccac

SEQ ID NO: 25 - protein, 736 aa, unidentified
  source 1..736: note = adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 599 aa, unidentified
  source 1..599: note = adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 534 aa, unidentified
  source 1..534: note = adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 738 aa, unidentified
  source 1..738: note = adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 29 - protein, 601 aa, unidentified
  source 1..601: note = adeno-associated virus
TAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 30 - protein, 535 aa, unidentified
  source 1..535: note = adeno-associated virus
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 31 - DNA, 2205 bp, synthetic construct
  source 1..2205: mol_type = other DNA
gctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 32 - DNA, 1794 bp, synthetic construct
  source 1..1794: mol_type = other DNA
gctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 33 - DNA, 1794 bp, synthetic construct
  source 1..1794: mol_type = other DNA
gctccgggtaagaagcgccccgtggaacagagcccccaggagccggatagtagcgcgggaattggtaaatctggagcgcaaccagctaagaaacgcttgaacttcggtcaaaccggagacacagagtccgtcccagacccgcagccaattggggaacctcctgctgcaccgtctggtgtcggttccttgactatggcttctggtggcggtgcgccggttgcagacaataatgagggtgctgacggggtgggttcaagtagcggaaattggcattgcgacagccagtggttgggggatagggttatcacaacttcaacgaggacgtgggctctgcctacttataacaaccacctttataaacagatcagcaacagcacatcagggggaagctccaatgataatgcgtacttcgggtactcaactccttggggatattttgatttcaatagatttcattgtcacttttccccgagggactggcagcgcttgatcaacaacaattggggtttcagaccgaaaagactgaatttcaaactctttaatatccaagtgaaagaagtaaccgataacaacggtgtgaaaactatagcgaataacttgacgtctactgtgcaggtatttactgacagcgactaccagcttccgtatgttctcgggtctgctcatgaaggatgcctcccacctttccccgccgacgtgtttatgatcccacaatacgggtatctgacgttgaacgatgggagccaagcggtgggaaggagttccttctattgccttgaatactttcctagccaaatgctgcggaccgggaacaatttccaatttagctatgagttcgaaaatgttcctttccacagtagttacgctcattcccagagtctcgataggcttatgaatcctctcatcgatcagtatctgtattacctctccaagactatcaacggctctggccaaaatcaacagacccttaaattctcagtagccggacctagcaatatggcagtccaggggaggaattatatacctggtcccagctatagacaacagagagtgagcacgactgtgacacagaacaataacagcgaattcgcgtggccaggcgcatcatcatgggcactcaatggccgcaacagtctcatgaatcccggtcccgccatggcgtcacataaggagggagaagaccgatttttcccgctttcaggatctctgattttcggaaaacaagggactggccgggataatgtcgacgccgataaagtgatgattaccaatgaagaagagataaagaccacaaacccggtagctaccgaatcttatggtcaggtagccacgaatcatcagtcagcccaagcgcaggcccaaaccggttgggttcaaaatcagggtatattgcctggtatggtgtggcaggaccgcgatgtatatttgcagggcccgatatgggcaaaaattccccataccgacgggaattttcacccatccccactcatgggagggttcggcatgaagcatcctcctccacagatcctcattaagaatacgccagttcccgctgacccaccaactgcttttaacaaggacaaactcaactcttttattactcaatattccacgggccaagtgagtgtagagatcgagtgggaattgcaaaaggaaaacagcaagcgctggaacccagagatccagtatacgagtaactattacaagagtaacaacgtggaattcgcggttaacactgaaggggtctattctgaaccacgacctatcggtacgcgataccttactagaaacctg

SEQ ID NO: 34 - DNA, 1599 bp, synthetic construct
  source 1..1599: mol_type = other DNA
gcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 35 - DNA, 1599 bp, synthetic construct
  source 1..1599: mol_type = other DNA
gctagtggtgggggagcgcccgttgccgataataacgagggtgcagacggtgttggctctagtagtggtaattggcattgcgatagccagtggctcggtgaccgggtcataacgacgtccactagaacttgggccctgccaacctataacaatcacttgtacaaacagatcagtaacagcacgtctgggggttccagcaatgacaacgcttacttcggttattcaactccttgggggtacttcgactttaaccgcttccattgtcatttcagcccgcgggattggcagcggcttatcaacaacaattggggatttcggcctaaacggctgaactttaaacttttcaatattcaagtgaaagaggtaacagacaacaatggggtaaagacaatcgcaaacaatcttacttccactgtccaggtgttcacagacagtgactatcaattgccatatgtgttgggctcagcacacgagggttgccttcctccttttccggctgacgtctttatgattccccagtatggttacttgacgctcaatgatggttcccaagcggtgggtagaagtagcttctattgtctggagtattttccttctcaaatgctgcgaaccggcaataattttcagttcagctacgagtttgagaatgtgccattccattcttcctacgcgcactctcagagcctcgaccgcctgatgaatcccttgatcgaccaatacctgtactatttgagcaagacgataaatggatcaggtcagaatcagcaaacgctgaagtttagcgtagctggcccgagtaatatggcggtacaaggcagaaattatatcccaggtccatcatatcggcagcaacgggttagtacgacggttacgcagaataataactccgaatttgcgtggccgggggcgagttcttgggcgttgaatggtcgaaattccctcatgaatcctggaccagccatggcgtctcacaaggaaggcgaggaccggttcttccccttgagtggctctttgatttttggtaagcaaggcactggtcgcgataacgttgatgctgataaagtgatgatcactaatgaagaagaaattaagacaacgaatccagttgcaacggaaagctatggacaagtagcaactaaccaccaaagcgcacaagcccaggcacaaacgggctgggtacaaaaccaggggattttgccaggcatggtatggcaagatagggacgtgtacctgcaaggtccaatttgggcaaaaatacctcataccgacgggaatttccacccttctcctctgatgggtggcttcggcatgaaacatccaccaccccagatccttattaaaaacacacctgtcccggcagacccgccgacggcttttaataaagacaaacttaactcttttataacacagtacagtaccggccaagtcagtgtagaaatcgagtgggagctgcaaaaggaaaacagcaagagatggaatcccgaaattcaatatacatccaattactataaaagtaacaatgtcgaatttgcggtgaatactgaaggtgtgtatagtgaaccaaggccgatcggaacacgatatctgacccgaaacttg

SEQ ID NO: 36 - DNA, 641 bp, synthetic construct
  source 1..641: mol_type = other DNA
gaaagatccaagagagacgagtagagattttttttttgggattgatgtttgtcgttctttgagttgtcgtcgagttacgccttttgtaagaatgttccgcaggagaggaggaggatgggcatgagtgagggtgagagggcttgcccgcttttttttttaaaaacgctgaagacgtggttgtcaaacaaaccccccatagaaacgattttgttacggtgcggtccagacgtcacttgaatggctccgcggaaaggccagggagggaaggggggagggaggaaacatgaaacatgttgaacggctcaacagggtttgggggacaagagaggtagcgccctgatggactgctccctcccctcctttccctcaatgtctcattcatccatgcttcccccttctctctctcccctccgttccatcccccgcgggcgtggtagtggcgtgatgggatccactaaaatgtacgtgtaagaaaagccggtgagcttacgcttttgtgaaagtgggagtacgagtgttgtgtgtgtgtgtagtggtttcagaccccagacagaggcgaagcagaaaaagcagacgatgaagacgacgaagaaatgagcagtctatttttatcgtggaaacagaagaggtgatatcgtctc

SEQ ID NO: 37 - DNA, 634 bp, synthetic construct
  source 1..634: mol_type = other DNA
ggccgccgacacctggtgtgtgtgggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctt

SEQ ID NO: 38 - DNA, 617 bp, synthetic construct
  source 1..617: mol_type = other DNA
tggccagggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctccctt

SEQ ID NO: 39 - DNA, 1001 bp, synthetic construct
  source 1..1001: mol_type = other DNA
atgggaaagaaaggatgagaaaggagaaaggacatctagataaccggcatatcacggtggtgtattagtgtagaatagtgaagagaagacttgggaaaatgtgtaggaaaggttgtttctgtgtatgtgggttgggatgggtggctgtttgagaaggaacagcgggcagggcgatgtagtgctgaacgggcacggaaccactgagactgaaggaagtagggagagagaggggcaggggacgtgcactttaatctttgcctcggtagagtatacccatgcaagagtatgtggccacctgtggtggcttttggccaggtctggtgcagtgccaatcatctcccatcaataatacaacttcagaacaacggcgcattgatggggagagagaaagtaaatttaagtaaggggtacgtagtagaggattcaactgaaatattttcgaggagcggttgggaagttgaccgattgaaaggagaagggaggggagcaggtgtgatagtcatgtgtaaagtaattcttttttgccgtcgtcacacaatccacatcaatgataaaatatgtttaaggatcaatcacacggagtcggtcataaggcaaccgcaaacgcaatgcaaactagcaagcaagcaggaccacaacaacaaccattaccatcacaggcgacagcagcagcagctgcagcagcagcaaggcaagcaaaggccatctgcgcgtggacttttccaggcaccggctttaagcgtaattttcaatgattgttgtccgtgtatcctctcaccctttcaccgcttgcgcgcacgtgagcagcacaactggtaatgcccgaggacaatagacgaggaaaaagaagaagaaaaatgaagagggagtgatgaagaagaagagaagaataaaagaagacagaaaagaagataaaaaagtcaaaagacgacaaagacggaaaaaaaagaaacgcgcaattttaacaccagcaacgacgacgaaaagggcgtcagctttgc

SEQ ID NO: 40 - DNA, 696 bp, synthetic construct
  source 1..696: mol_type = other DNA
tttggaagagagtttgctgtttgtaagagaaaagggaaaggggaaaactaatcgtcaggaggtatgatatggatataaagagtgcgccgtggaaagaaaactagagttgtttcattcctttgattccatttaagcaagcaacagcaacgccaacagcaaccgcatttagaaaaagatagagggaacagtctgagtagagagaatacaagtagagcggaagtgatagagtgaggcaagagggaagtatctgcggggcaggagatgggggacagattgcgcatcataaaaggagtagaaatgccattggttagcgtcgctttcctctgccctctctgtcctcaaattcctatgcacccacacttctcggtcccgtgttccacgcatgccttcccccgtgtctccctctccgccttaccctctcccccatctttatccattattagaaatagttgtgtcgagtcgatcggatcgcagaagtatgtgtacagagtgaggggggagggagaagaagacgacggaagttaagaggacggacatgatgacggagtagaaaaagaaacgcaagcacaccagcagcggtagaaacttttaaaaaaagaccaaggactggaaatctgtaatcttggcgaagacaaaaaaatccacctacttaatctctgaatcaagaaaataaaacactcactcccctcggtttta

SEQ ID NO: 41 - DNA, 646 bp, synthetic construct
  source 1..646: mol_type = other DNA
accgtcggttggtcggagaagaaggaaggatggagggaaggagcgaaggaaggtcaatgtaccgatagagtctttctcacttatctgaaataagggaggaggaggacgggataatcaaaagcaatcagcagggcaattatataatttggagaaaagcaacatccaactgagcgtctccttgcgtgtgtttcagcctgtgtcggatgtccggcgcgggagtgatggcgcttgcttgtcttgatttggatgcgtgtgaatgtgctctgtgtaggtgatagttcaggcaggggttatatttgatagtgacgctctgtgtaagtgatagaattgcagaggctatatatggtggggaacgccgagaagtgtatgcgggataagcagtggctgttgtctgtctcagatacagaccatatcccctctcatttattttttaaacaaggacctttttctcaaagaaataggaggggaaagaaaagagtactgcctgctttaaataaaaaagtgcaaccctgtccttgggaaagaaacttagcaagagagaaagcgcaaaaaacacatgaacagccctgcgacaaggacgtgattttatcgagtcctttgaacaaaatgcataaaaaaaaaagccgagacgcgaaccgagtaacaa

SEQ ID NO: 42 - DNA, 593 bp, synthetic construct
  source 1..593: mol_type = other DNA
tagagcgaatgagggaggtgagggaggggaagtacgaatgtagtcgagtcgtggggttccgtttaatgtcttttcttgagaaataaaggtgggtcggtatggaagcgatgaggaggtttggaaagcgaaatttacagcgattgtgtggcccgttctgccagacggagacgacgccggcttcactctcggccgcgtttttttttgggtcctgccgccacaatgatagagacgacgctgtcttctctagggcgcgggaatcctgccgcaacaacaaaagatgagagtatgaaaaatcgatgataatcaatatgagagagagaaaaggagtacatgggaagcaggaatatatacattcactcgtgttccagcgttgtgtgccatacggttataccgcttcgcatgtccttacgtttcacctgatgacataacatgccttttgtgctccttcttcgatcgtaaattccttcatttcaccatcaagaattcacactctacttgatgctgtaccatctccatcgccttgcacgttcacaccgccctccgtaacaatttgccaccagaggcggccccccttaccagagccatgaactcgg

SEQ ID NO: 43 - DNA, 471 bp, synthetic construct
  source 1..471: mol_type = other DNA
aggggggatcgggagggaggaaaggagggaggaacaaggagtttgctttaggtgggattgagaatgagtgtgtgggggggctatagtaagagaacaaggatttctgtttggaagtcaaggaatctagtattttgagttatggaagagtttgacggaaagtgttgtgactgaaaggcgatcagggtagaaaacacgatcaaacgtgaagaactcggacgagataataagagcagaagagaggcgagaggaagagggaagtggaagatgattgtggtggtatttatgccgtcacaagcgacagacagcagagaaaaaaggggcggaagaatgaagtgacgacgacacgctttgcctgtatttgcttggcccgtttctctcccgtaggcactcattcgcccaccccgaaacggtgtgttttggggaagagtgttacccctccccccatccttttgactccacaggagaactaag

SEQ ID NO: 44 - DNA, 227 bp, synthetic construct
  source 1..227: mol_type = other DNA
tagagtgttaggaggaagggagggacggagaggagggtggaccccgtggatgagtaggggtgtctttccttgaggagtgagtgagaggaaatgattttgtgtagtgagtgacaacgaaacaaacgaggaagacgaccattgcgaattcgagtttccaacgaacgaagacatgaaagaagcacagagagggaatacatgtttgttagagagtatgcgcggccttttcc

SEQ ID NO: 45 - DNA, 763 bp, synthetic construct
  source 1..763: mol_type = other DNA
agggagaagggtgagagggaggaatggagggaggagggagctcgggcaaggcatggttatggatgcagattgatagcgccgccttacgtttgctaatgtttatgattaggggaagggcaccaaaatagacgagccagccccacctagcaagagaagagagtagccatagacaccgcagcaatagcagcagtaccgggacgcgcttccctatgttggatacaggtaagccctgcacgtgtgtcatgcatgaaggatagcaagaacgaggccgggccactatttccagcagcagactccagaaaaggccattttgggatgtaacttcattttgtatcaagagtggaagggaaaggaaaagaacaagagagaggaaagggcgaaggacacagcagagatagtgagtgagataaagggtgtacccactttttgggatgtacgacatggtgaaagagggacatgacataaaaatagagaaaatagaaggcgccgcttccttagtgaattcggtgggaagaagatctttgggagtccttgggaggggaacaagagggaaaaaggagataacatcagagattccatgagagtaacagattcacggatgtggcattaggcaaaggtgccgaaattaggataggacgtcgcaacaaataaagatatgaaaaggaagacttccactagacaagagaaggcgacgtttccttaaatatgtctgtatcatattttccgcgcaccttttatattgtattcttttgatcgaacgagt

SEQ ID NO: 46 - DNA, 983 bp, synthetic construct
  source 1..983: mol_type = other DNA
gatgcgcagtatcaggttgtagggcttctgtttaggatggggtagcggaggaggggatcgtgcgtgaagggcgctgttttcagaggcggaggcggtggctacacgcccgtctgaagcacaagcgaagcactgctgcgccgggccgcgcgtgggcatatttttatgtcagacgcaaggtctctaaaagtgagaaagggccgtcattaagtaagggcgcaagatcgggcctagggcacatactctcccgatatgtgtaaatggccgcttttgtgcctccagcacgcgtgcaaagtgtcaccaaagcgcacgacgcaatggcagtagactccagtgaaatgggtaaaagcggcatgtcattaggtgctttggaaaaaacaatgccaagctttcataaagaagcaaacaagcaagacaaatcataatacccccactcaagagtaactggagatgaccacgttggctaagtccggttgaaaatcggcgggttttcaatttatgtcgacatccctgccccggttcgcgggtcaccatggaaacatactttttttgttgcgccacttctcggccgtaccccacaagacgtatccacgattgcgaaaggtaaagttctatttcatgtcccctggtcgactggtttgtgtaactccttgccaaaaacctacctgttactttatcaccgacggcgttggcacaacgggcactgcaccaaaaaaccgccgcctcgattgggggccgcgaacatgacgtgtcggaacaggaaaagcaaccgctgcaaggcgccggatcttagcgcgcagtcgtagcagcaggaatcgacaatatgtgttctcgtttctgcatacgtcgactctggacagctatgactgacccgactctgaccacacttcatgacccacccaccacacaacgagggcaggtgccgcagcagctttagcacgttgctcctctaccagactcagccctaaaacccgcctttagacaag

SEQ ID NO: 47 - DNA, 674 bp, synthetic construct
  source 1..674: mol_type = other DNA
taggcgtgtgtctttggcttgtttttcttttgttcttttcgctcttccccccactacaagctggtgtcttaattgcacgcgtctctgaagtcttgagcttttgtctcgtgctccacttttaggacgacgaccacggcaagtgtcatgggacgttaagccctggtgtgcgctgaaacttgtacagcagagctttgctttgcatgtgtagcatcccagcttgtgcacttaattgctcggggtcgtttatttctctgccagccttggacctcgtggttgggcgtttcctcctccttggtgcactgaacctgtccgcatcctgcgtcggtttggcggtcttttgtcctttcctctatagcccgcccgtctagagggcacacgcgatgatctttatatctcttcatgtgtctttgttttaactaggatactgccgggtgaatgcccatcagacaagaggccaaactctatctacacccttttgacttctgttgtggtcgtagtgtgtgcttgcatgccctgaaagtccaggcatcccacttgtgctctaaccccattcaaaacagcagaagtgcctaattaagatatagattcatgatctcctgtcccctccttcttaccttttcacaaacctcacacagaagtctccactcttcgcctctaaaacctctttttaaatt

SEQ ID NO: 48 - DNA, 1167 bp, synthetic construct
  source 1..1167: mol_type = other DNA
gttacttcttgagagagtggtggagttgactatcgtgtggagtgtttggggagggaaagggcggagcgtgagtgcaatgcgaggtgggcgaagtgggcatgtgataaatggctgtgtggtggaggccttcgctgcgtgtctgtgactgtcttgattgtgtgcttagagtgagataccaaagcaagatcttccctgccatcccttcattgtcccacgggccgaagagatggggggcttgacgagaggacagggatgcaggtgcgatgcggtcctgtccatggggcaggaaccgctggggtgcagtggcacagaagacagaaggagaaaacacatgcaccaaataaacatatgacaaagagtcaagcagtagtcaaaacaaccaaaacgtaagcaagacggaacaagatggcacgcgtctgcaacagaccggctcgcgccgaacgtgcctcctgcttttcaacgatcctgcgaggtcaaccaggatttgctcgccgggacgatttcatccccttatcaacgagcccttgaggctccaggcgtgcttccacaccccagttggtaacaggacattggggcatcttgcctatcttgtcttagtgccgaaagcctcaacgacctcccatggggtctgctcaacgcctcaaccttgcagtaaggatccccgagggcaagacccgcaaagccttctgtcgtcggacaaagcggagcgagggaacaggctcagctcaaccctcttgagagcccataagtgccccctgatctatcttcaacagtctttccctgtcacaagaaaacccagctagttgaccaagttgctagagctgataccttgtacttcgctctttgtgtgctttacctgattggacatggacagacctccccttgctcttccttctaggagcctgggctctcgctcttgttctttcgagagacctttcccttgagttgcgtatccagcgatcaagtatgaagagtgctttcaaacctagatacgttctgcccagttctcttgcccttttccacacgtgctccacatcttcacacgactcgcaccatacccgacgaaacccctcaaaacatcgcaacacttacatcccgctcgtgtcccacccccgatgccatatcctctacagcagcagcaccaccaccaccacttcttaagttaac

SEQ ID NO: 49 - DNA, 1379 bp, synthetic construct
  source 1..1379: mol_type = other DNA
gttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagcaacaccaaacagtttcgacttggcggcatcttctcggtgccataacaaacactgagaaagcctttggactgatcctggcactcgttgccgtgtcattccatctccaattcggacctccaatcaagcaaggctacccgctgaatttaagcatataactaagcggaggaaaagaaactaaccaggattcccctagtaacggcgacaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcag

SEQ ID NO: 50 - DNA, 993 bp, synthetic construct
  source 1..993: mol_type = other DNA
gtatagctacatggtagctagtagtcgtcaaaggaaataaagacatagcgtaaagtatacaaaatgacttccgcggcgcgggagcggcaggcagcacttgcaaaacgaagcccaatgggccagattacggtcaacctcgtcattttattacggtcgtcagcaagagcggtttagaccgtcgccggcacgggtggctcccgatattcttggctttcgtgggtggaggcggcgtgtgactcctcatctttaagtagtggtcgcttttgctgcatgcttttgatcacctcgtgattgatggctggattctcagctgtcttctgacatctttgggtgcatgggttgaccggtgtcgcgggaggcagaccctgttccgtggaggtgtgtggctgcggatgcggcgacacggaagaagggcgtgcatcacttctctcttagtcgatttacgatgcgcttctgcgcacagctcattcacccacccgccaccactcctcttgcacatgtatgcaggtgtcgggccaagttagtcgcgcgctgagtggcccgaaccccgcatgtggtagtggaatgcgtggtcggcagaagccttgagcacagcgaaagaagaaaacaagataagcaggggatggctgcggtttgcgcgtgcctttgccgcgcagcccgtcacaacaatacgtatccctcatgggaattacactcacctgtttcggcgcgcaggtgggacatgcctcgttcagcatgagacgcaaggcttctcgttgttgggccggacggcggcgtggcttctttgctgcactgccggcgctgcaccacctcgccctgccagccaccggccagggtcgtggcatcgatgggcatgtacgcgcctcacgcacggcccgcgccggcattttgttcataggactttgataaacacctctggctcattctccctctcctcctcacccatccaccacgagcacagcacccccacccctcaaccctcacttcgtaaca

SEQ ID NO: 51 - DNA, 789 bp, synthetic construct
  source 1..789: mol_type = other DNA
attgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 52 - DNA, 1020 bp, synthetic construct
  source 1..1020: mol_type = other DNA
aaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaa

SEQ ID NO: 53 - DNA, 384 bp, synthetic construct
  source 1..384: mol_type = other DNA
ccactgagccaggaagaatctactctcatcgagcgcgcgacagctactatcaacagcattcctatcagcgaggactactcagtcgcaagcgcagctctgtcttcagatggaaggatctttacaggggtgaatgtgtaccactttaccgggggtccgtgcgcagaactcgttgttctgggcacagctgccgcagctgctgcaggaaacctgacctgtatcgtggccatcggcaacgaaaatcggggaatcctttcaccttgtggaagatgtcgccaggtacttttggatcttcatccaggaatcaaggcgatcgtgaaagattcagacggacagccaactgcggtagggatcagagaacttcttcccagcggctacgtatgggaa

SEQ ID NO: 54 - DNA, 369 bp, synthetic construct
  source 1..369: mol_type = other DNA
gccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggac

SEQ ID NO: 55 - DNA, 789 bp, synthetic construct; identical to SEQ ID NO: 51
  source 1..789: mol_type = other DNA

SEQ ID NO: 56 - DNA, 789 bp, synthetic construct; identical to SEQ ID NO: 51
  source 1..789: mol_type = other DNA

SEQ ID NO: 57 - DNA, 591 bp, synthetic construct
  source 1..591: mol_type = other DNA
cttgctacacagagccaatcccagaccttgaatcagagcgaaaacctgcctcaaccaccccaggtgtgggatctgcttcagtggcttcaggttgtggcccaccagtggcagactatcaccaaggtcccgatggagtgggtcgtgcctcgagagatcggaattgcaatccctaatggatggggcacagaaagttcaccgcctgctcctgaaccaggaccatgcccgccaactacgattacctctacaagcaagtctcctacagctcacctcgaggacctgcaaatgaccacaccaacaagcgcaacagcccctcctggtggaattctgacttccactgactccactgccacgtctcaccatgtgacagggtcagacagttcaactactacgggggattctggtctcagcgattctacctccagttcctcaactttcaggtcaaaacggctgcgcacaaccatggaatcccgcccaagtcccatcaccctccccgctcggagcagatcttcccgaactcagaccatcagcagcaggacctgctcaggcagacttacaagagcggctagcaggcggtcacagagaacattctct

SEQ ID NO: 58 - DNA, 615 bp, synthetic construct
  source 1..615: mol_type = other DNA
gattacaaggatgacgacgacaaacttgctacacagagccaatcccagaccttgaatcagagcgaaaacctgcctcaaccaccccaggtgtgggatctgcttcagtggcttcaggttgtggcccaccagtggcagactatcaccaaggtcccgatggagtgggtcgtgcctcgagagatcggaattgcaatccctaatggatggggcacagaaagttcaccgcctgctcctgaaccaggaccatgcccgccaactacgattacctctacaagcaagtctcctacagctcacctcgaggacctgcaaatgaccacaccaacaagcgcaacagcccctcctggtggaattctgacttccactgactccactgccacgtctcaccatgtgacagggtcagacagttcaactactacgggggattctggtctcagcgattctacctccagttcctcaactttcaggtcaaaacggctgcgcacaaccatggaatcccgcccaagtcccatcaccctccccgctcggagcagatcttcccgaactcagaccatcagcagcaggacctgctcaggcagacttacaagagcggctagcaggcggtcacagagaacattctct

SEQ ID NO: 59 - DNA, 618 bp, synthetic construct
  source 1..618: mol_type = other DNA
tacccctacgatgttcctgattatgcaatggctacccaatcccaatctcaaacactgaaccaatccgaaaatctcccacagccacctcaagtttgggatctcctccagtggttgcaagtcgtagcacaccagtggcagaccataacaaaggtccctatggagtgggttgtaccaagggagatcggaatcgccatccccaacgggtggggaaccgagtcatcaccacctgctcccgaacctggaccctgtccacccacaacaatcacatccacatccaagagtcccaccgcgcatctcgaagacctccagatgaccactcctacttctgcaacagcgcctcccggtggcatcctcacgtctacggattctaccgccacttctcaccatgtaacaggctccgatagctccactactactggcgattcaggactgagcgactctacgtctagctcctctacctttaggagtaaacggctgcggactactatggagagtagaccttctcccataactctgcctgctagatctcgctccagccggacacagaccatttccagtagaacatgttccggacgcctcacacgggctgcatctaggcggtcacagcggaccttttcc

SEQ ID NO: 60 - DNA, 1188 bp, synthetic construct
  source 1..1188: mol_type = other DNA
gaacttgtaggctggcttgttgacaaaggcattacgtcagagaagcagtggattcaagaagaccaggcttcatatatctcattcaatgctgcttcaaattccagaagccaaatcaaggcagcacttgataacgctggtaaaattatgtccctgacgaagactgctcccgactacttagtcggtcagcaacctgttgaagatatttcatccaataggatatacaaaatactcgaactcaacggatacgaccctcaatacgctgccagcgtttttttgggctgggcaacaaagaagttcggaaagagaaatacaatctggctcttcggaccagcaactacgggaaagactaacattgcagaggccattgcacatactgtccctttttatggatgtgtgaactggacgaacgaaaatttccccttcaatgattgcgttgacaagatggttatttggtgggaagaagggaaaatgacggctaaggtcgtcgagagtgctaaagcaattctcggagggtccaaggtccgcgtggaccagaagtgcaaaagtagcgctcagattgaccctacgcctgttatagttacttccaacacgaatatgtgtgcagtgattgacggcaattccacaacttttgaacaccaacagccacttcaggataggatgttcaagttcgaacttactagaaggttggaccatgattttggcaaagttacaaaacaggaggtgaaagatttttttaggtgggcaaaagaccatgtagtagaagtcgaacacgagttctacgtcaaaaaaggtggtgccaagaaacgacctgcaccaagtgatgcagacatctccgagcctaagagagtgagagagagtgttgctcaaccatcaacgtctgatgctgaagcatctatcaactacgcagacagataccaaaacaagtgctcacgtcatgtgggcatgaatttgatgcttttcccttgtcggcagtgcgaaagactgaatcaaaattctaatatctgcttcactcatggcgtaaaggattgccttgagtgctttcctgtttccgaatctcaacctgtgagtgtggtcaagaaggcttatcagaagttgtgctacatccaccacatcatgggcaaggtgccagatgcatgtaccgcttgcgatttggtgaatgttgaccttgatgactgtgtgtccgagcag

SEQ ID NO: 61 - DNA, 1860 bp, synthetic construct
  source 1..1860: mol_type = other DNA
cctggcttttacgaaatcgtaatcaaagtgcccagtgaccttgatgagcacctgcccggtatttctgattcattcgtgaactgggtggctgaaaaagagtgggaactccctcccgactccgacatggatttgaacctcattgagcaggccccactgacagtggcggaaaaactccaacgagatttcttgaccgagtggcgaagagtttccaaagcacccgaggctctcttctttgttcaatttgagaaaggagaatcatacttccacatgcatgttttggtcgagacaacaggtgtgaagtcaatggtgctgggacgcttcctgagccagatccgcgaaaaactcatccagcgcatttatcgcggcatagaacccaccctccccaattggtttgcggttacaaaaaccagaaacggtgccggtggtggaaacaaagttgtggacgagtgttatatccctaattatcttctgcccaagacacagccggaattgcagtgggcctggacaaatatggaacagtacctgtctgcctgcctcaacctcacagaacgcaagaggcttgtcgcccagcatcttacacatgtctcacaaacccaggagcagaacaaggaaaatcagaatccaaattctgacgccccagtgattcgctctaaaaccagtgctagatatatggaactggtagggtggctggttgacaaaggaatcacctccgagaagcagtggattcaggaagaccaggcttcctacatttccttcaacgctgcttctaacagccgcagccagatcaaagcagcgttggacaacgccgggaagataatgtctctgacaaaaactgcccctgattacttggtcggtcagcaacctgtagaggacatctcctctaacagaatctataagattctggaactgaacggatacgatccgcagtatgctgcgtctgtgtttctgggttgggctactaagaaatttgggaagcggaacactatctggttgtttgggccagccactacagggaagacaaacatcgcagaagccatcgctcacacggtcccgttttacggatgcgtgaactggacgaatgaaaatttccccttcaacgactgtgtcgataaaatggtcatctggtgggaagagggaaagatgaccgccaaagtggtggagagtgcgaaggctatcctgggtggctccaaagtgcgcgtcgatcagaagtgcaagtcatcagcccagatagacccaacacccgtgattgtgaccagtaacaccaacatgtgcgccgtgatcgacggaaactctacaactttcgaacaccagcagcctttgcaggacaggatgttcaagtttgagttgacccggcggctggatcatgatttcggaaaggtgaccaagcaagaagtaaaagacttctttaggtgggcaaaagaccacgttgtcgaggtcgagcacgagttctacgtgaaaaaagggggtgctaagaagcgacctgcccccagcgatgccgatatttcagaaccaaagcgagtcagagagtcagtagcgcaaccaagtacctccgatgcagaggcgagtatcaattacgcggaccgctatcaaaacaagtgttcccgacatgtaggcatgaacttgatgttgttcccttgccgccaatgtgagaggttgaaccagaacagcaacatctgcttcacacacggggtgaaagactgcctcgaatgctttcctgtgtccgagtcacagcccgtttctgtggtcaagaaggcgtatcagaagttgtgttatattcaccatataatgggaaaggttcccgacgcctgtactgcatgtgatctcgtcaacgtggatttggatgactgcgtttccgagcag

SEQ ID NO: 62 - DNA, 1584 bp, synthetic construct
  source 1..1584: mol_type = other DNA
gcaagcagggaagaggaacaaagagagaccactcccgaacgcggcagaggagcagccaggagacctccaacgatggaagacgtcagttctccttcccccagccctcctcccccaagagcacctcctaagaaaaggctcaggcggagacttgagtcagaggacgaagaggactccagccaagatgctctcgttccacgcacaccatcacccaggccatctaccagcacagccgacctcgccatcgcatctaaaaaaaagaagaaacggccaagcccaaagcctgaaagaccaccctcccccgaagtgatagtagactctgaggaggaaagagaggacgtggctctccagatggtaggtttctcaaacccaccagttctcatcaagcatgggaaagggggaaaaagaaccgtacggaggctcaatgaggatgaccccgttgcaaggggcatgaggactcaggaagaaaaggaggagtcttccgaggcagagtctgagtccactgttatcaatcccctgagtctgcccatcgtcagtgcatgggagaaggggatggaagccgccagggcactgatggacaaataccatgtggataacgatttgaaagcaaacttcaaacttctgccagaccaagtggaagcattggccgcagtttgcaagacctggctgaatgaggaacatcgaggtctccaactgacttttacttccaacaagacattcgttaccatgatgggacgatttctccaagcctatctccagtcattcgccgaggtgacgtacaagcaccacgaacctacaggctgtgccttgtggttgcaccgctgtgccgaaatcgagggagaactgaaatgcctccacgggagcatcatgataaacaaggagcacgtaatcgagatggacgttacaagcgagaatggtcaacgcgccctcaaggagcaatcaagcaaggccaaaatcgtgaaaaaccgctggggaaggaacgtcgtccagattagtaatacagacgcccgctgctgtgtacacgacgctgcttgccccgcaaaccagttctctgggaaaagttgtgggatgttcttttccgagggagccaaggcccaggtggccttcaagcaaatcaaagcatttatgcaggccctctacccaaacgctcagacaggccacgggcatttgcttatgccactgcggtgtgaatgcaactcaaagccagggcacgcacccttcctcgggagacaattgcccaaacttaccccctttgctctgtccaacgccgaggacctggacgcggacctcatcagtgacaaaagcgtgctggcctcagtccatcatcctgctctcatcgtcttccagtgctgtaaccccgtgtacagaaacagcagagcccagggcgggggtccgaattgtgacttcaagatttctgcgcctgacctgctgaatgctctcgttatggttcggtcactctggagcgagaattttaccgaactccctcgcatggtagtgcccgagttcaaatggagcactaaacaccagtacagaaatgtctctctcccagttgcgcatagtgacgcccgccagaaccccttcgacttc

SEQ ID NO: 63 - DNA, 879 bp, synthetic construct
  source 1..879: mol_type = other DNA
acgaccagcggtgttcctttcggcatgactctcagacccacccgctcaaggctgagtaggcggaccccatactcccgcgacagactgccaccatttgaaactgaaacccgagcgaccattttggaggaccaccccctgttgccagagtgtaacactctgaccatgcacaatgtttcatacgtcagggggctcccttgcagcgtcggattcaccttgattcaggagtgggtcgtcccttgggacatggttctgaccagagaagaactcgtcatactcaggaagtgtatgcatgtgtgtctgtgctgcgccaatatcgacatcatgaccagcatgatgattcacggctacgagtcttgggctctgcattgccattgctcatctccgggcagccttcagtgcatagcaggtgggcaagttctcgcatcctggttccgaatggtcgtggatggtgcaatgttcaaccagcggtttatctggtacagagaggtggtcaattacaacatgccaaaggaagtgatgtttatgtcctcagtgttcatgaggggacggcacctcatctatctgcggctttggtatgatgggcacgtgggaagcgtagttcctgccatgtcatttggatactctgccctgcattgtggcatcttgaacaatattgtcgtgctgtgttgtagttattgtgcggacctctccgaaattagagttcgatgctgcgctcgaagaactcgaagactgatgttgcgggccgtccgaataatcgccgaggagacgacggctatgctgtacagttgcaggacagagcggcggaggcagcagtttataagagccctgcttcagcaccataggcccattcttatgcatgactacgactccacacccatg

SEQ ID NO: 64 - DNA, 2613 bp, synthetic construct
  source 1..2613: mol_type = other DNA
gctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctg

SEQ ID NO: 65 - DNA, 714 bp, synthetic construct
  source 1..714: mol_type = other DNA
gtgagcaaaggtgaagaactctttacgggagtcgtaccgatattggtggaattggacggtgacgtgaacgggcacaaatttagcgttcgcggtgaaggcgagggagacgccacaaacgggaaactcactctgaaatttatctgcaccacaggaaaactgcctgtcccttggcctacactggttacgacgtttgggtacggcgtggcctgtttctctcggtatcccgaccacatgaagcagcacgacttcttcaagtctgctatgcccgagggctacgttcaggaacgcaccatctcattcaaagacgacggcacctataagacgcgggcagaggtcaagttcgagggcgacacccttgtcaaccgcatagaactgaagggcatcgatttcaaagaggacggcaatattctcgggcacaaactggaatacaatttcaacagtcactacgtgtatatcaccgccgataagcaaaagaattgcatcaaggccaacttcaagatacgccataacgtggaagacgggtccgtgcaattggccgaccattaccagcagaacacacctattggcgacgggccagtattgcttcccgataatcactacctgtcccaccaatccaaactgtccaaagaccctaatgaaaagcgagaccacatggtgctgctcgaatttgtcaccgcggctgggattactcacggcatggatgaactctacaaa

SEQ ID NO: 66 - DNA, 1143 bp, synthetic construct
  source 1..1143: mol_type = other DNA
aggtaaggagtagggagggggagagagaaataggagagcgagaatgagcggaaggtgagcaagaggaagtagttatacgacagcattagaagcagcgagaggcaaccgagaaattgctggggggagcggcaaggttctaagaaattggtaagagaaccaaaatcaatagggcaacgcattgtcctgaagaagaaataagaaccaaaccctgcgacagcaatgctttgttttgaaaaacaattgcttgtacctgctgacactccacttaagcctacccatgctgccactgctattgctaccgccactgcaaccatcccgactacggttgcccttttctcctcggcaacgagccataagagggcaccgccaactcaacaactcaaaaacagaccccttgacccaccctcccaacccaatcccacttgagggccagggcggtgcggagcttttgacttttaccctccgagtcctcaccgtctgcgtggccttccggaggatggatccaacggcacgcgcgataagtgccccccttccaccagcccgcatggcggcgacggcatggcgggccttgacgaggaatgaggcctgactcgatgataagcagaggcagggcgagtgctgcgacttacaccagcttgacttgccacggcatcaggaagcaccgaagtgctaggtggagacgcgatggccgtggtcgtgaggttcatgccgcagaaggagcggaggcgctactcctgttgcagcgctatcactgttgcagcactatatgtaacaacttattgattccatcgttctgtacaaagtgcatctttggtgttcaattgggaaaggaacgtgagacaaggccacatcgttctcacaggcctgcatgacgaaaactatgcaggcggcggaaaaagagcaagggaggctgcgtaacacaacgagaacaaaacaaaaagtgtgactggcatttttgtgtgtgttaacagctggtcgcagtgatgggaatcaaaatgcaaaaaggaaaaagggagaagaacgaaagtacgcaggcctacaacgtgtgagcgtcttcatttccacgacgtctcatcacacgaattgtccatcatgtattctagcgatactcgaaaagaattcaagagaagaagaaagagctta

SEQ ID NO: 67 - DNA, 1213 bp, synthetic construct
  source 1..1213: mol_type = other DNA
tcatcgtcatcgtcctttcccgtacgagaaatttaaaagctagacctctacttaccccctactattaacccgctcatcctgacctctcactcctcttcctcatcttcctccaccaccggcacaccctccacttctccttcgtgccgtacaggtaacaaagccaacctctcgagcacttctaacgctgggcccttggggttgccatggaccagggcctctatctgtcgacgttgagagcgtgtcatggatagctcaaaatcaattatcacatctactaccgacagggcattcttccagctttcactaattagcaaactctctagcaatgtcacgagaatatccgccccagcgacatcatctcgatctcgcatctcttgcagcaagcccgcaacccgatccaggcgacctcgatccacgtgacagagcgctgctactgctgcagggtccccgccctgaaatgctggaggcaaattgtgatatacatcaaatgccttctcaatctgctttggattcgccgcccagcatccacggagcaaagacccatagaaccctcggtccgccttccacccctgttcctccaccaggccgaggatggtttcagctgcggccaggtcaggcggatctagcttggtgcaatagacaaacgtggcccaccggtaagtgtcataacttgtgtttggcttcaagaccaagtcccccagcagatctctcgcccggtccgactggccaacgcgtaaggaggcgagaatcgcagggacacctaagcgatcatgttttaccagcatctcccgcggtaaggtagcagccacgtccgccagctcctggaagcgacatgcttgcttcagacacttgaaaagccggcggtaggtcagtgggttcggcatcgaggatggcccatggctgagcatttcgtgatatagacgcaaagccctctccgacatgcccgtcgggtcatcacgcaccaaacggtaggtcacgtacatcgcctcattgtacatgatcgtgttgggggtacgcccactcgctttgaatgtctcaagcaattctaaggcctcacgtcggcttttcacatctgccttggccaacgcatgcatgaatgcaccgaacatgatatcattcgaagcctcttccggatcgcccgccaacgttctgaagcgcgtcagaaggccattcgcctgcgtgcgattcctcaaatccgccgtcacactaatca

SEQ ID NO: 68 - DNA, 508 bp, synthetic construct
  source 1..508: mol_type = other DNA
gacccaggaggaggtgagggagctcttgcaggcgttacgggcgagtgtgcgatggacggaaggagggcgggaggaaggcgccctcaaggaggagcaggacggaggggcagcgccaatggaggcggcagagtgaggaaaggagactgctctgtcgtgttttgaatggacagaaacaggaaaaaatgggagagagagtgaagagggcagaaggagagagataatgtgagtggaggtgcctgttggtgtgtgtgtccgcggcaagagagaaaacgaaaaagagagtaaaatactgctcacgcagagagtagggcggcgaaagagaaggagaacagagcgtcgatgtacatttttgtgttgatacattcagcgagagccccccccaaaacaaaaggggatagtgtaacaggaggaataatcttctgcttgtctgaagagagagaaaaagagagccgagaaaagagggcggaaagcagaggtgtacagaacaagaaatccaagccaagccatg

SEQ ID NO: 69 - DNA, 533 bp, synthetic construct
  source 1..533: mol_type = other DNA
atgtcctcttgcttctcttgcatgcgatttgattttatctttctcacgtctgattgcttttcccaccttttgcttacttacatttagatcttttgtttttagcgtctgtggcctcattgccctttctaatccaccaacccctccgcaatcgccccgacaccagcatcagcatcagcatcagcaccctcccttcctctcccctcctcctcctcctcccctaaaagccctttttctgcttctgtgaccgagggcctttctgtcctcctcgtgcctggaagtccatcaaccctctcgcgattgcagcctgcatcggagtcatcatatccatatcctcaatcgtcccatccagcacccccgcatgaattcccgccccgagagcaacggcctcgtcaggattaaccgttacacgcggctttcgccccgtgactgcttcaaccaagcgctgtatcgagggcatcctggtcgcccctcccaccataaccacctcctcgatcactctcccctgcgggaactcccttatcttcaaccccggg

SEQ ID NO: 70 - DNA, 504 bp, synthetic construct
  source 1..504: mol_type = other DNA
gatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagtacaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgat

SEQ ID NO: 71 - DNA, 1425 bp, synthetic construct
  source 1..1425: mol_type = other DNA
gtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccgctacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

SEQ ID NO: 72 - DNA, 84 bp, synthetic construct; identical to SEQ ID NO: 12
  source 1..84: mol_type = other DNA

SEQ ID NO: 73 - DNA, 1797 bp, synthetic construct
  source 1..1797: mol_type = other DNA
acggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 74 - DNA, 21 bp, synthetic construct
  source 1..21: mol_type = other DNA
cttgctacacagagccaatcc

SEQ ID NO: 75 - DNA, 27 bp, synthetic construct
  source 1..27: mol_type = other DNA
tacccctacgatgttcctgattatgca

SEQ ID NO: 76 - DNA, 588 bp, synthetic construct
  source 1..588: mol_type = other DNA
gctacccaatcccaatctcaaacactgaaccaatccgaaaatctcccacagccacctcaagtttgggatctcctccagtggttgcaagtcgtagcacaccagtggcagaccataacaaaggtccctatggagtgggttgtaccaagggagatcggaatcgccatccccaacgggtggggaaccgagtcatcaccacctgctcccgaacctggaccctgtccacccacaacaatcacatccacatccaagagtcccaccgcgcatctcgaagacctccagatgaccactcctacttctgcaacagcgcctcccggtggcatcctcacgtctacggattctaccgccacttctcaccatgtaacaggctccgatagctccactactactggcgattcaggactgagcgactctacgtctagctcctctacctttaggagtaaacggctgcggactactatggagagtagaccttctcccataactctgcctgctagatctcgctccagccggacacagaccatttccagtagaacatgttccggacgcctcacacgggctgcatctaggcggtcacagcggaccttttcc

SEQ ID NO: 77 - DNA, 16245 bp, synthetic construct
  source 1..16245: mol_type = other DNA
aggtaaggagtagggagggggagagagaaataggagagcgagaatgagcggaaggtgagcaagaggaagtagttatacgacagcattagaagcagcgagaggcaaccgagaaattgctggggggagcggcaaggttctaagaaattggtaagagaaccaaaatcaatagggcaacgcattgtcctgaagaagaaataagaaccaaaccctgcgacagcaatgctttgttttgaaaaacaattgcttgtacctgctgacactccacttaagcctacccatgctgccactgctattgctaccgccactgcaaccatcccgactacggttgcccttttctcctcggcaacgagccataagagggcaccgccaactcaacaactcaaaaacagaccccttgacccaccctcccaacccaatcccacttgagggccagggcggtgcggagcttttgacttttaccctccgagtcctcaccgtctgcgtggccttccggaggatggatccaacggcacgcgcgataagtgccccccttccaccagcccgcatggcggcgacggcatggcgggccttgacgaggaatgaggcctgactcgatgataagcagaggcagggcgagtgctgcgacttacaccagcttgacttgccacggcatcaggaagcaccgaagtgctaggtggagacgcgatggccgtggtcgtgaggttcatgccgcagaaggagcggaggcgctactcctgttgcagcgctatcactgttgcagcactatatgtaacaacttattgattccatcgttctgtacaaagtgcatctttggtgttcaattgggaaaggaacgtgagacaaggccacatcgttctcacaggcctgcatgacgaaaactatgcaggcggcggaaaaagagcaagggaggctgcgtaacacaacgagaacaaaacaaaaagtgtgactggcatttttgtgtgtgttaacagctggtcgcagtgatgggaatcaaaatgcaaaaaggaaaaagggagaagaacgaaagtacgcaggcctacaacgtgtgagcgtcttcatttccacgacgtctcatcacacgaattgtccatcatgtattctagcgatactcgaaaagaattcaagagaagaagaaagagcttagttcggaaactatcgatagggttttggcagttcaactcctcataagtttttctttcaccgtgggcaaaatcggctgtattgctgcccatcacaagacgccctggattcctcccttgtcctcacttcatgagaaggagatgagtgtgtgggagcacagcatttctgcttcgtcatgaccactgagaacgaattgttctgcatgaatgtttgcaatacaatgcgttcttggagatgagagcttctcgacatctcctgccataaacacatgcgcagttgacaagagagcaccaggggctgaggacggaatggtacgtgtgaccgagagatcgaatacgcatccagatccccactcagttctcctgtcgaagacttgcttcgatcagtgaagatcacctggccctttttctttgtaaaaggcaaggcccagggcggtcggaaggtgttgtattggtacgctttgacgtagccttctcgttgatactttgattggcatacgaaacttggtgggacggcttgataaccggctgttgcaacaaggactatgcatcgaaggtgcacaccctggaagccccgagtcctctgttcgagatactgctccatcacacgagcaggactcacaacgagagttcaatcaatcgggtcagcttcgtatgagtgatgcacctggtgaagttgcagagaggcatcggaagaaggtgctttttgacactgtcacgagcgtgtgtccatgagtcatggcacatctgatctcgtgaccaccttttcattcttacggagaatgagaaacgtctcttgaaaaaatttgctgtccaatcagtggcgatgcggtgttcttgaacacgcttgccctgactgaaccattgaagttgtgtgatgtgtgaagatgaatgtcttcaccataactcaacaacaataacgatagcaacaccaaacagtttcgacttggcggcatcttctcggtgccataacaaacactgagaaagcctttggactgatcctggcactcgttgccgtgtcattccatctccaattcggacctccaatcaagcaaggctacccgctgaatttaagcatataactaagcggaggaaaagaaactaaccaggattcccctagtaacggcgacaaccagctcagaactggagcggacaaggggaatccgactgtttaattaaaacaaagcattgcgatggtcggagacgatgttgacgcaatgtgatttctgcccagtgctctgaatgtcaaagtgaaggaattcaaccaagcgcgggtaaacggcgggagtaactatgactctcttaaggtagccaaatgcctcgtcatctaattggaatgcccctcccccctccctccttcccttcatcctcccctccgagcagatggctagtggtgggggagcgcccgttgccgataataacgagggtgcagacggtgttggctctagtagtggtaattggcattgcgatagccagtggctcggtgaccgggtcataacgacgtccactagaacttgggccctgccaacctataacaatcacttgtacaaacagatcagtaacagcacgtctgggggttccagcaatgacaacgcttacttcggttattcaactccttgggggtacttcgactttaaccgcttccattgtcatttcagcccgcgggattggcagcggcttatcaacaacaattggggatttcggcctaaacggctgaactttaaacttttcaatattcaagtgaaagaggtaacagacaacaatggggtaaagacaatcgcaaacaatcttacttccactgtccaggtgttcacagacagtgactatcaattgccatatgtgttgggctcagcacacgagggttgccttcctccttttccggctgacgtctttatgattccccagtatggttacttgacgctcaatgatggttcccaagcggtgggtagaagtagcttctattgtctggagtattttccttctcaaatgctgcgaaccggcaataattttcagttcagctacgagtttgagaatgtgccattccattcttcctacgcgcactctcagagcctcgaccgcctgatgaatcccttgatcgaccaatacctgtactatttgagcaagacgataaatggatcaggtcagaatcagcaaacgctgaagtttagcgtagctggcccgagtaatatggcggtacaaggcagaaattatatcccaggtccatcatatcggcagcaacgggttagtacgacggttacgcagaataataactccgaatttgcgtggccgggggcgagttcttgggcgttgaatggtcgaaattccctcatgaatcctggaccagccatggcgtctcacaaggaaggcgaggaccggttcttccccttgagtggctctttgatttttggtaagcaaggcactggtcgcgataacgttgatgctgataaagtgatgatcactaatgaagaagaaattaagacaacgaatccagttgcaacggaaagctatggacaagtagcaactaaccaccaaagcgcacaagcccaggcacaaacgggctgggtacaaaaccaggggattttgccaggcatggtatggcaagatagggacgtgtacctgcaaggtccaatttgggcaaaaatacctcataccgacgggaatttccacccttctcctctgatgggtggcttcggcatgaaacatccaccaccccagatccttattaaaaacacacctgtcccggcagacccgccgacggcttttaataaagacaaacttaactcttttataacacagtacagtaccggccaagtcagtgtagaaatcgagtgggagctgcaaaaggaaaacagcaagagatggaatcccgaaattcaatatacatccaattactataaaagtaacaatgtcgaatttgcggtgaatactgaaggtgtgtatagtgaaccaaggccgatcggaacacgatatctgacccgaaacttgtaaggccgccgacacctggtgtgtgtgggatcaggaggagggagtgaagaggagaagggatctggtttcagagatccccacttctgccgtcgtctttcggccttccttccttttaggtgtcatgccttaggtccttcaagtcctcacctgtcgtcgtcatgtgtgtgtgtgcccgtcatacaagtcactcgatccaattcacgcatcggttcaatcaaaataagactagaccccgagggaagaagggcagaaggaaatcgaaggggtgggatgtgtgtgagagagggaaggagaaatgaaagaagtgaacaatgtcatggtagccagtaaggagagagtagaagcgaagaaagcaaaagcactgttgtgaagaaacgaaatggaagatggtcatcgctcctggctctacttgtggtttttctatctttaatttcaggcgtcctggactcgttacatcagctcccttatctcattggtttatcccctactctactgctgcttcttccttccatccgtgactgtataacaacgaattgtagtaccgcagatagacaatagaaaaatgccaaaaaaggcatcattgatttgctcctccccattaagtcactgtacgccaccatcgccactaccctcccttgtatagctacatggtagctagtagtcgtcaaaggaaataaagacatagcgtaaagtatacaaaatgacttccgcggcgcgggagcggcaggcagcacttgcaaaacgaagcccaatgggccagattacggtcaacctcgtcattttattacggtcgtcagcaagagcggtttagaccgtcgccggcacgggtggctcccgatattcttggctttcgtgggtggaggcggcgtgtgactcctcatctttaagtagtggtcgcttttgctgcatgcttttgatcacctcgtgattgatggctggattctcagctgtcttctgacatctttgggtgcatgggttgaccggtgtcgcgggaggcagaccctgttccgtggaggtgtgtggctgcggatgcggcgacacggaagaagggcgtgcatcacttctctcttagtcgatttacgatgcgcttctgcgcacagctcattcacccacccgccaccactcctcttgcacatgtatgcaggtgtcgggccaagttagtcgcgcgctgagtggcccgaaccccgcatgtggtagtggaatgcgtggtcggcagaagccttgagcacagcgaaagaagaaaacaagataagcaggggatggctgcggtttgcgcgtgcctttgccgcgcagcccgtcacaacaatacgtatccctcatgggaattacactcacctgtttcggcgcgcaggtgggacatgcctcgttcagcatgagacgcaaggcttctcgttgttgggccggacggcggcgtggcttctttgctgcactgccggcgctgcaccacctcgccctgccagccaccggccagggtcgtggcatcgatgggcatgtacgcgcctcacgcacggcccgcgccggcattttgttcataggactttgataaacacctctggctcattctccctctcctcctcacccatccaccacgagcacagcacccccacccctcaaccctcacttcgtaacaatggctccgggtaagaagcgccccgtggaacagagcccccaggagccggatagtagcgcgggaattggtaaatctggagcgcaaccagctaagaaacgcttgaacttcggtcaaaccggagacacagagtccgtcccagacccgcagccaattggggaacctcctgctgcaccgtctggtgtcggttccttgactatggcttctggtggcggtgcgccggttgcagacaataatgagggtgctgacggggtgggttcaagtagcggaaattggcattgcgacagccagtggttgggggatagggttatcacaacttcaacgaggacgtgggctctgcctacttataacaaccacctttataaacagatcagcaacagcacatcagggggaagctccaatgataatgcgtacttcgggtactcaactccttggggatattttgatttcaatagatttcattgtcacttttccccgagggactggcagcgcttgatcaacaacaattggggtttcagaccgaaaagactgaatttcaaactctttaatatccaagtgaaagaagtaaccgataacaacggtgtgaaaactatagcgaataacttgacgtctactgtgcaggtatttactgacagcgactaccagcttccgtatgttctcgggtctgctcatgaaggatgcctcccacctttccccgccgacgtgtttatgatcccacaatacgggtatctgacgttgaacgatgggagccaagcggtgggaaggagttccttctattgccttgaatactttcctagccaaatgctgcggaccgggaacaatttccaatttagctatgagttcgaaaatgttcctttccacagtagttacgctcattcccagagtctcgataggcttatgaatcctctcatcgatcagtatctgtattacctctccaagactatcaacggctctggccaaaatcaacagacccttaaattctcagtagccggacctagcaatatggcagtccaggggaggaattatatacctggtcccagctatagacaacagagagtgagcacgactgtgacacagaacaataacagcgaattcgcgtggccaggcgcatcatcatgggcactcaatggccgcaacagtctcatgaatcccggtcccgccatggcgtcacataaggagggagaagaccgatttttcccgctttcaggatctctgattttcggaaaacaagggactggccgggataatgtcgacgccgataaagtgatgattaccaatgaagaagagataaagaccacaaacccggtagctaccgaatcttatggtcaggtagccacgaatcatcagtcagcccaagcgcaggcccaaaccggttgggttcaaaatcagggtatattgcctggtatggtgtggcaggaccgcgatgtatatttgcagggcccgatatgggcaaaaattccccataccgacgggaattttcacccatccccactcatgggagggttcggcatgaagcatcctcctccacagatcctcattaagaatacgccagttcccgctgacccaccaactgcttttaacaaggacaaactcaactcttttattactcaatattccacgggccaagtgagtgtagagatcgagtgggaattgcaaaaggaaaacagcaagcgctggaacccagagatccagtatacgagtaactattacaagagtaacaacgtggaattcgcggttaacactgaaggggtctattctgaaccacgacctatcggtacgcgataccttactagaaacctgtaatttggaagagagtttgctgtttgtaagagaaaagggaaaggggaaaactaatcgtcaggaggtatgatatggatataaagagtgcgccgtggaaagaaaactagagttgtttcattcctttgattccatttaagcaagcaacagcaacgccaacagcaaccgcatttagaaaaagatagagggaacagtctgagtagagagaatacaagtagagcggaagtgatagagtgaggcaagagggaagtatctgcggggcaggagatgggggacagattgcgcatcataaaaggagtagaaatgccattggttagcgtcgctttcctctgccctctctgtcctcaaattcctatgcacccacacttctcggtcccgtgttccacgcatgccttcccccgtgtctccctctccgccttaccctctcccccatctttatccattattagaaatagttgtgtcgagtcgatcggatcgcagaagtatgtgtacagagtgaggggggagggagaagaagacgacggaagttaagaggacggacatgatgacggagtagaaaaagaaacgcaagcacaccagcagcggtagaaacttttaaaaaaagaccaaggactggaaatctgtaatcttggcgaagacaaaaaaatccacctacttaatctctgaatcaagaaaataaaacactcactcccctcggttttagcaaagctgacgcccttttcgtcgtcgttgctggtgttaaaattgcgcgtttcttttttttccgtctttgtcgtcttttgacttttttatcttcttttctgtcttcttttattcttctcttcttcttcatcactccctcttcatttttcttcttctttttcctcgtctattgtcctcgggcattaccagttgtgctgctcacgtgcgcgcaagcggtgaaagggtgagaggatacacggacaacaatcattgaaaattacgcttaaagccggtgcctggaaaagtccacgcgcagatggcctttgcttgccttgctgctgctgcagctgctgctgctgtcgcctgtgatggtaatggttgttgttgtggtcctgcttgcttgctagtttgcattgcgtttgcggttgccttatgaccgactccgtgtgattgatccttaaacatattttatcattgatgtggattgtgtgacgacggcaaaaaagaattactttacacatgactatcacacctgctcccctcccttctcctttcaatcggtcaacttcccaaccgctcctcgaaaatatttcagttgaatcctctactacgtaccccttacttaaatttactttctctctccccatcaatgcgccgttgttctgaagttgtattattgatgggagatgattggcactgcaccagacctggccaaaagccaccacaggtggccacatactcttgcatgggtatactctaccgaggcaaagattaaagtgcacgtcccctgcccctctctctccctacttccttcagtctcagtggttccgtgcccgttcagcactacatcgccctgcccgctgttccttctcaaacagccacccatcccaacccacatacacagaaacaacctttcctacacattttcccaagtcttctcttcactattctacactaatacaccaccgtgatatgccggttatctagatgtcctttctcctttctcatcctttctttcccatctaagagaatgttctctgtgaccgcctgctagccgctcttgtaagtctgcctgagcaggtcctgctgctgatggtctgagttcgggaagatctgctccgagcggggagggtgatgggacttgggcgggattccatggttgtgcgcagccgttttgacctgaaagttgaggaactggaggtagaatcgctgagaccagaatcccccgtagtagttgaactgtctgaccctgtcacatggtgagacgtggcagtggagtcagtggaagtcagaattccaccaggaggggctgttgcgcttgttggtgtggtcatttgcaggtcctcgaggtgagctgtaggagacttgcttgtagaggtaatcgtagttggcgggcatggtcctggttcaggagcaggcggtgaactttctgtgccccatccattagggattgcaattccgatctctcgaggcacgacccactccatcgggaccttggtgatagtctgccactggtgggccacaacctgaagccactgaagcagatcccacacctggggtggttgaggcaggttttcgctctgattcaaggtctgggattggctctgtgtagcaagtttgtcgtcgtcatccttgtaatccatgggtcccgggttctcctccacgtcaccggcctgcttcagcaaggagaagttcgtcgctcccggtccctggaaggacagggtcgtcatgccgcttcccgtacgttcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatctgtggatggagggagggaaggagggggggagtgagtgagtcgagagaacgacgactggggcaaagagaggatgacgccactgcaacaaaagacagggaagaatagaggctgttggaaaacaggaacacagtgaagaatagaggctgcagggtaacaggcacatttatcgcagagaggcgggcggcaggggcgatacgtaaagagacccctaaacttctccgcgtcatccgttcacgctacctccttccctccctttactcccattgatcaccaaggcagcgcccgcagccaccagcagccaccgtcctcagtgtcacagctagacgcgtccccatcaggcagcagaccaccagaatatgcgctttcgcttcccatcatatccctccctcctccccttctattcctcatcatgcatcatttctttctccttgctgcatgcccctcttcttaccttggctaagatctaccggaggaagaagtcttcaacgatagataaatgcctgtatcgtgatggttgtttgaatggtggaagtgagccaggtattgagaacgaggggcgcaagggtgcgtggaagaaggcagggagaggcgaggcgaaggagaagggcatgtggtgggaacgaagcctccacaccacagccacccatgcttttctctgttcgagttcccgggttctactctcacacactacacgtgcacagaaaacattcacgcacaacagagatacagagggatccgtcgagatggtctacacgtcctaatgtcccctgctttcgaagcccagagccatcgctttctgtttttatccattcgcaacacatcctgttctcgcgacctctcctcttcctggccaccatcgcttctccgtgccatgccctgtacatccttccctccctcgttgtctaccacactaatcatgcgctctgcagcagacactcggctactatttgtgtcgtacggattgaggggagggaggggaagggaggcaagaagtaaatcggtggtcaacaacagacgcggtctccccaccctccctcattcctttcccctcctataatgagcgaagaaagccatggacaccgagtcacccactctcctaatccaccaccaccagggcactaacggactcacgctcaccaaactcggtaacaatatccactgcacacagatacacacgcaaccatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagaaagatccaagagagacgagtagagattttttttttgggattgatgtttgtcgttctttgagttgtcgtcgagttacgccttttgtaagaatgttccgcaggagaggaggaggatgggcatgagtgagggtgagagggcttgcccgcttttttttttaaaaacgctgaagacgtggttgtcaaacaaaccccccatagaaacgattttgttacggtgcggtccagacgtcacttgaatggctccgcggaaaggccagggagggaaggggggagggaggaaacatgaaacatgttgaacggctcaacagggtttgggggacaagagaggtagcgccctgatggactgctccctcccctcctttccctcaatgtctcattcatccatgcttcccccttctctctctcccctccgttccatcccccgcgggcgtggtagtggcgtgatgggatccactaaaatgtacgtgtaagaaaagccggtgagcttacgcttttgtgaaagtgggagtacgagtgttgtgtgtgtgtgtagtggtttcagaccccagacagaggcgaagcagaaaaagcagacgatgaagacgacgaagaaatgagcagtctatttttatcgtggaaacagaagaggtgatatcgtctctcatcgtcatcgtcctttcccgtacgagaaatttaaaagctagacctctacttaccccctactattaacccgctcatcctgacctctcactcctcttcctcatcttcctccaccaccggcacaccctccacttctccttcgtgccgtacaggtaacaaagccaacctctcgagcacttctaacgctgggcccttggggttgccatggaccagggcctctatctgtcgacgttgagagcgtgtcatggatagctcaaaatcaattatcacatctactaccgacagggcattcttccagctttcactaattagcaaactctctagcaatgtcacgagaatatccgccccagcgacatcatctcgatctcgcatctcttgcagcaagcccgcaacccgatccaggcgacctcgatccacgtgacagagcgctgctactgctgcagggtccccgccctgaaatgctggaggcaaattgtgatatacatcaaatgccttctcaatctgctttggattcgccgcccagcatccacggagcaaagacccatagaaccctcggtccgccttccacccctgttcctccaccaggccgaggatggtttcagctgcggccaggtcaggcggatctagcttggtgcaatagacaaacgtggcccaccggtaagtgtcataacttgtgtttggcttcaagaccaagtcccccagcagatctctcgcccggtccgactggccaacgcgtaaggaggcgagaatcgcagggacacctaagcgatcatgttttaccagcatctcccgcggtaaggtagcagccacgtccgccagctcctggaagcgacatgcttgcttcagacacttgaaaagccggcggtaggtcagtgggttcggcatcgaggatggcccatggctgagcatttcgtgatatagacgcaaagccctctccgacatgcccgtcgggtcatcacgcaccaaacggtaggtcacgtacatcgcctcattgtacatgatcgtgttgggggtacgcccactcgctttgaatgtctcaagcaattctaaggcctcacgtcggcttttcacatctgccttggccaacgcatgcatgaatgcaccgaacatgatatcattcgaagcctcttccggatcgcccgccaacgttctgaagcgcgtcagaaggccattcgcctgcgtgcgattcctcaaatccgccgtcacactaatca

SEQ ID NO: 78-79 - skipped (2 entries)

SEQ ID NO: 80 - DNA, 611 bp, synthetic construct
  source 1..611: mol_type = other DNA
gaaagatccaagagagacgagtagagattttttttttgggattgatgtttgtcgttctttgagttgtcgtcgagttacgccttttgtaagaatgttccgcaggagaggaggaggatgggcatgagtgagggtgagagggcttgcccgcttttttttttaaaaacgctgaagacgtggttgtcaaacaaaccccccatagaaacgattttgttacggtgcggtccagacgtcacttgaatggctccgcggaaaggccagggagggaaggggggagggaggaaacatgaaacatgttgaacggctcaacagggtttgggggacaagagaggtagcgccctgatggactgctccctcccctcctttccctcaatgtctcattcatccatgcttcccccttctctctctcccctccgttccatcccccgcgggcgtggtagtggcgtgatgggatccactaaaatgtacgtgtaagaaaagccggtgagcttacgcttttgtgaaagtgggagtacgagtgttgtgtgtgtgtgtagtggtttcagaccccagacagaggcgaagcagaaaaagcagacgatgaagacgacgaagaaatgagcagtctattttta

SEQ ID NO: 81-84 - skipped (4 entries)

(19) United States
(12) Patent Application Publication
(10) Pub. No.: US 2026/0297502 A1
(43) Pub. Date: Oct. 1, 2026

(54) NANNOCHLOROPSIS-PRODUCING VIRUSES AND METHODS AND COMPOSITIONS FOR MAKING THE SAME

(71) Applicant: Insmed Incorporated, Bridgewater, NJ (US)

(72) Inventors: Anthony BERNDT, Bridgewater, NJ (US); Allan KASPAR, Bridgewater, NJ (US); Brian KASPAR, Bridgewater, NJ (US); Samit VARMA, Bridgewater, NJ (US); Gretchen THOMSEN, Bridgewater, NJ (US)

(73) Assignee: Insmed Incorporated, Bridgewater, NJ (US)

(21) Appl. No.: 19/480,661

(22) PCT Filed: May 3, 2024
(86) PCT No.: PCT/US2024/027680

§ 371 Date: Oct. 31, 2025

Related U.S. Application Data
(60) Provisional application No. 63/500,546, filed on May 5, 2023.

(51) Int. Cl. C12N 1/12; A61K 39/23; A61K 48/00; C07K 14/015; C12N 15/864; C12R 1/89

(52) U.S. Cl. CPC C12N 1/12; A61K 39/23; A61K 48/0058; A61K 48/0091; C07K 14/015; C12N 15/8645; C12N 2750/14132; C12N 2750/14134; C12N 2750/14143; C12N 2750/14152; C12N 2830/34; C12R 2001/89

(57) ABSTRACT

The present disclosure relates in part to Nannochloropsis microalgae that produce recombinant viruses. Expression cassettes, transgenes and methods for producing recombinant viruses in the Nannochloropsis are also provided. In particular, methods and materials are provided for producing recombinant adeno associated viruses (AAV) in Nannochloropsis. In some embodiments described herein, the Nannochloropsis is Nannochloropsis oceanica.

### CROSS REFERENCE TO RELATED APPLICATION

This application claims priority from U.S. Provisional Patent Application Ser. No. 63/500,546, filed May 5, 2023, the disclosure of which is incorporated by reference in its entirety.

### REFERENCE TO AN ELECTRONIC SEQUENCE LISTING

The contents of the sequence electronic listing (INMD_192_01WO_SeqList_ST26.xml; Size: 139,860 bytes; and Date of Creation: May 3, 2024) are herein incorporated by reference in its entirety.

### BACKGROUND

Microalgae are unicellular, eukaryotic, photosynthetic microorganisms which have been successfully utilized to produce a variety of human recombinant proteins. The promise of algal systems, for the mass production of biologic therapeutics, lies in their inherent low cost of goods and capitalization costs. With their simple production process and faster growth rate, microalgae can be grown in as little as four weeks at the flask scale, with the potential to scale up to 64,000 liters in another four to six weeks. Being phototrophic organisms, transgenic microalgae are suitable for growth in indoor photobioreactors as well as outdoor raceway ponds. With the help of various tools of genetic manipulation, stable transgenic lines of microalgae can be generated in as little as ten days and further scaled up to large production volumes within few weeks. Microalgae also possess complex post-transcriptional modification pathways and thus can produce properly glycosylated proteins.

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka (1992). Current Topics in Microbiology and Immunology 158:97-129.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

In recent years, the use of AAV as a gene therapy vector has been the subject of increasing interest. This is because of the ability of AAV to infect both dividing as well as non-dividing cells and to target multiple cell types within peripheral tissues and central nervous system, and its relatively low risk of pathogenicity to humans. Despite these advantages AAV-based gene therapy, like other virus-based approaches, faces the need for large scale vector production to enable human administration at an affordable price.

Mammalian expression systems, for example systems in HEK293 cells, are the current industry standard for gene therapy and vaccine vector production. HEK293 cells are grown either in adherent or suspension manner. Adherent culture systems suffer from the inability to be readily scaled up. This restriction can be overcome to a limited extent by utilizing suspension culture systems. Recently, baculoviral expression systems in insect cells are also being investigated for obtaining higher yields of viral vector production, although this system is also restricted due to its genetic and physical instability. Thus, all of these systems suffer from high production costs which are directly proportional to the scale of production.

To address the issues of scalability and cost-effectiveness, alternative methods for gene therapy vector and vaccine vector production are needed in the art. The present disclosure addresses this and other needs.

### SUMMARY

In one aspect of the disclosure, an expression cassette is provided, comprising, (i) a first Nannochloropsis-endogenous promoter comprising a first end and a second end, (ii) a first gene coding sequence operably linked to the first end of the first Nannochloropsis-endogenous promoter, (iii) a first Nannochloropsis-endogenous 3′ untranslated region (UTR) downstream of the first gene coding sequence and operably linked thereto, and (iv) an antibiotic resistance gene coding sequence. The antibiotic resistance gene coding sequence is operably linked to the first Nannochloropsis-endogenous promoter or a second Nannochloropsis-endogenous promoter, and the antibiotic resistance gene coding sequence is operably linked to the first Nannochloropsis-endogenous 3′ UTR or a different Nannochloropsis-endogenous 3′ UTR, and the first gene coding sequence is an adeno-associated virus (AAV) cap coding sequence.

The AAV cap coding sequence, in one embodiment, is a VP1, VP2 or VP3 coding sequence. In a further embodiment, the AAV cap coding sequence is an AAV9 VP1, AAV9 VP2 or AAV9 VP3 coding sequence.

In one embodiment, the expression cassette further comprises an assembly activating protein (AAP) coding sequence operably linked to the first Nannochloropsis-endogenous promoter, or operably linked to a second Nannochloropsis-endogenous promoter. The AAP coding sequence, in one embodiment, is an AAV9 AAP coding sequence.

In one embodiment of the expression cassette, the antibiotic resistance coding sequence is a zeocin, neomycin, G418, kanamycin, rifampicin, benomyl, nystatin, spectinomycin, ampicillin, apramycin, hygromycin B or chloramphenicol resistance coding sequence.

In one embodiment of the expression cassette comprising a cap coding sequence, the first Nannochloropsis-endogenous 3′ UTR comprises a lipid droplet surface protein (LDSP) 3′ UTR. In another embodiment, the first Nannochloropsis-endogenous 3′ UTR comprises a heat shock protein 3′ UTR; a cellulose synthase (CS) 3′ UTR, a violaxanthin chlorophyll a-binding protein 1 (VCP1) 3′ UTR or a VCP2 3′ UTR.

In another embodiment of an expression cassette comprising a cap coding sequence, the first Nannochloropsis endogenous promoter is a Nannochloropsis endogenous bidirectional promoter. In a further embodiment, the bidirectional promoter is the ribosomal subunit (Ribi) bidirectional promoter, the nitrate reductase (NR) bidirectional promoter, or a violaxanthin chlorophyll a-binding protein (VCP) bidirectional promoter. In a preferred embodiment, the Nannochloropsis-endogenous promoter is the ribosomal subunit (Ribi) bidirectional promoter. The Ribi bidirectional promoter, in one embodiment, comprises a nucleic acid sequence of SEQ ID NO:1 or SEQ ID NO:2.

In yet another embodiment of an expression cassette comprising a cap coding sequence, the first Nannochloropsis-endogenous promoter is the ubiquitin extension protein (UEP) promoter, a violaxanthin chlorophyll a-binding protein (VCP) promoter, the β-tubulin (β-tub) promoter, the lipid droplet surface protein (LDSP) promoter, the elongation factor (EF) promoter, the ribosomal Pol I promoter, or the ribosomal subunit bidirectional promoter (Ribi).

In another embodiment of an expression cassette comprising a cap coding sequence, the expression cassette further comprises a viral 2A (2A) peptide coding sequence downstream of the first gene coding sequence, and a second gene coding sequence downstream of the 2A peptide coding sequence and upstream of the first Nannochloropsis-endogenous 3′ UTR, and operably linked thereto. The second gene coding sequence is operably linked to the first end of the first Nannochloropsis-endogenous promoter, and is the antibiotic resistance coding sequence, an AAV cap coding sequence, or an AAV AAP gene coding sequence. In addition, the second gene coding sequence is different from the first gene coding sequence.

In yet another embodiment of an expression cassette comprising a cap coding sequence, the first Nannochloropsis-endogenous promoter is a bidirectional promoter, and the expression cassette further comprises a third gene coding sequence operably linked to the second end of the bidirectional promoter, and a second Nannochloropsis-endogenous 3′ UTR downstream of the third gene coding sequence and operably linked thereto. The third gene coding sequence is the antibiotic resistance coding sequence, an adeno-associated virus (AAV) cap coding sequence or an AAV assembly activating protein (AAP) gene coding sequence, and the third gene coding sequence is different from the first and second gene coding sequences.

In a further embodiment of an expression cassette comprising a cap coding sequence and a bidirectional promoter as the first Nannochloropsis-endogenous promoter, the expression cassette comprises a 2A peptide coding sequence downstream of the third gene coding sequence, and a fourth gene coding sequence downstream of the second 2A peptide coding sequence and upstream of the second Nannochloropsis-endogenous 3′ UTR. The 2A peptide coding sequence and the fourth gene coding sequence are each operably linked to the second end of the bidirectional promoter and the fourth gene coding sequence is the antibiotic resistance coding sequence, an adeno-associated virus (AAV) cap coding sequence or an AAV assembly activating protein (AAP) gene coding sequence. The fourth gene coding sequence is different from the first, second and third gene coding sequences.

In one embodiment of an expression cassette comprising a cap coding sequence and a bidirectional promoter as the first Nannochloropsis-endogenous promoter, the expression cassette further comprises a second Nannochloropsis-endogenous promoter operably linked to a fifth gene coding sequence, a 3′ UTR downstream of the fifth gene coding and operably linked thereto, and the fifth gene coding sequence is different from the first, second, third and fourth gene coding sequences. In a further embodiment, the second promoter is a ribosomal Pol I promoter, β-tubulin (β-tub) promoter or an elongation factor (EF) promoter. The fifth gene coding sequence, in one embodiment, is an AAV VP coding sequence or an AAV AAP coding sequence.

In a further embodiment of an expression cassette comprising a cap coding sequence, a bidirectional promoter as the first Nannochloropsis-endogenous promoter, and a second Nannochloropsis-endogenous promoter, the expression cassette comprises a third promoter operably linked to a sixth nucleic acid coding sequence, and a 3′ UTR is downstream of the sixth gene coding and operably linked thereto. The sixth gene coding sequence is different from the first, second, third, fourth and fifth gene coding sequences. In a further embodiment, the third promoter is a unidirectional promoter. In a further embodiment, the third promoter is a Pol I promoter, β-tubulin (β-tub) promoter or an elongation factor (EF) promoter. The sixth gene coding sequence, in one embodiment, is an AAV VP coding sequence or an AAV AAP coding sequence.

The Nannochloropsis endogenous nucleic acids in the expression cassettes provided herein, in one embodiment, are endogenous to Nannochloropsis oceanica.

In another aspect of the disclosure, an expression cassette comprising two Nannochloropsis-endogenous bidirectional promoters is provided. The expression cassette comprises, (i) a constitutively active Nannochloropsis-endogenous bidirectional promoter comprising a first end and a second end, (ii) a first gene coding sequence operably linked to the first end of the constitutively active Nannochloropsis-endogenous bidirectional promoter, wherein the first gene coding sequence is a Rep52 coding sequence, (iii) a first Nannochloropsis-endogenous 3′ untranslated region (UTR) downstream of the first gene coding sequence and operably linked thereto, (iv) a second gene coding sequence operably linked to the second end of the constitutively active Nannochloropsis-endogenous bidirectional promoter, wherein the second gene coding sequence is selected from an AAV assembly activating protein (AAP) gene coding sequence, and an antibiotic resistance gene coding sequence, (v) a second Nannochloropsis-endogenous 3′ untranslated region (UTR) downstream of the second gene coding sequence and operably linked thereto, (vi) an inducible Nannochloropsis-endogenous bidirectional promoter comprising a first end and a second end, (vii) a third gene coding sequence operably linked to the first end of the inducible Nannochloropsis-endogenous bidirectional promoter, wherein the third gene coding sequence is a Rep78 coding sequence, (viii) a third Nannochloropsis-endogenous 3′ untranslated region (UTR) downstream of the third coding sequence and operably linked thereto, (ix) a fourth gene coding sequence operably linked to the second end of the inducible Nannochloropsis-endogenous bidirectional promoter, wherein the fourth gene coding sequence is selected from an adenoviral gene coding sequence and an antibiotic resistance gene coding sequence, and (x) a fourth Nannochloropsis-endogenous 3′ untranslated region (UTR) downstream of the fourth coding sequence and operably linked thereto. The expression cassette comprises a single antibiotic resistance gene coding sequence.

In a further embodiment of an expression cassette comprising two Nannochloropsis-endogenous bidirectional promoters, the first, second, third and fourth Nannochloropsis-endogenous 3′ UTR are each selected from the phosphoglycerate kinase (PGK), pyruvate dehydrogenase (PDH), 1,3-β-D-glucan synthase (β-GS) and the Redoxin 3′ UTR.

The fourth gene coding sequence, in one embodiment of an expression cassette comprising two Nannochloropsis-endogenous bidirectional promoters, is an adenovirus E4orf6 coding sequence or a DNA binding protein (DBP) coding sequence.

In even embodiment of an expression cassette comprising two Nannochloropsis-endogenous bidirectional promoters, the expression cassette further comprises an adeno-associated virus (AAV) transgene comprising a promoter operably linked to a therapeutic gene coding sequence, wherein the promoter and therapeutic gene coding sequence are flanked by a 3′ AAV inverted terminal repeat (ITR) and a 5′ AAV ITR.

In one embodiment of an expression cassette provided herein, the expression cassette comprises a left homology arm and a right homology arm so that the expression cassette may be incorporated into a gene specific locus. The homology arms flank each end of the expression cassette. In one embodiment, the homology arms are designed for expression cassette integration into the ribosomal DNA (rDNA) locus docking-site. In a further embodiment, the homology arms comprise the nucleic acid sequences of SEQ ID NOS: 66 and 67.

Yet another aspect of the disclosure is directed to a Nannochloropsis packaging cell comprising at least one of the expression cassettes provided herein.

The present disclosure in yet another aspect, relates in part to a Nannochloropsis microalgal system for producing recombinant viruses, e.g., recombinant adeno-associated viruses (rAAVs). The rAAVs have utility, in some embodiments, as gene therapy or vaccine vectors. In some embodiments, a Nannochloropsis microalgae host is transformed with an expression cassette provided herein, and the microalgae is subjected to conditions suitable for culture, expansion, and production of a recombinant virus, e.g., a recombinant adeno-associated virus (AAV). In some embodiments, the rAAV encapsidates a therapeutic transgene.

In yet another aspect of the disclosure, a Nannochloropsis microalga that produces a recombinant adeno-associated virus (rAAV) is provided. The Nannochloropsis microalga in one embodiment, comprises one or more of the expression cassettes provided herein. In a further embodiment, the Nannochloropsis microalga comprises an adeno-associated virus (AAV) transgene comprising a promoter operably linked to a therapeutic gene coding sequence, wherein the promoter and therapeutic gene coding sequence are flanked by a 3′ AAV inverted terminal repeat (ITR) and a 5′ AAV ITR. In a further embodiment, the AAV transgene is an AAV9 transgene. The rAAV, in one embodiment, is a viral vaccine vector or a viral gene therapy vector, e.g., an AAV9 gene therapy vector.

In even another aspect of the disclosure, a method of producing a recombinant virus is provided. The method comprises, growing one of the Nannochloropsis packaging cells provided herein. The recombinant virus, in one embodiment, is a viral vaccine vector or a viral gene therapy vector.

### BRIEF DESCRIPTION OF THE DRAWINGS

FIG. 1 shows various diagrams of expression cassettes of the disclosure that employ unidirectional promoters (*Pr: unidirectional promoter; GCS: gene coding sequence; UTR: untranslated region; 2A CS: viral 2A peptide coding sequence). Expression cassettes of FIG. 1 in one embodiment, are stacked together to form more complexed cassettes.

FIG. 2 shows various diagrams of expression cassettes for use herein that employ a bidirectional promoter (*Bi-Pr: bidirectional promoter; GCS: gene coding sequence; UTR: untranslated region; 2A CS: viral 2A peptide coding sequence).

FIGS. 3-5 show various diagrams of expression cassettes for use herein that employ one or more Nannochloropsis-endogenous promoters, including one or more Nannochloropsis-endogenous bidirectional promoters (*Bi-Pr: bidirectional promoter; GCS: gene coding sequence; UTR: untranslated region; 2A CS: 2A peptide coding sequence).

FIG. 6 provides the architecture of the expression cassettes IN100, IN101, IN102 and IN103. CS: coding sequence; Hyg: hygromycin B; viral 2A: viral 2A peptide coding sequence; RPS15: 40S ribosomal protein S15; Ribi Prom.: ribosomal subunit bidirectional promoter; RPL12e: large ribosomal subunit protein uL11; LDSP: lipid droplet surface protein; UTR: untranslated region.

FIG. 7 is an image of a western blot showing anti-green fluorescent protein (GFP) immunoreactivity in N. oceanica clones transformed with pIN100, that also expressed tdTomato protein. Western Blot Map: Lane 1—Pageruler Plus Protein Ladder, Lane 2—Wild-Type untransformed N. oceanica, Lane 3—5×10⁹ vector genomes of AAV9, Lanes 4-12—Individual, genetically unique, transformants of vector pIN100 driving tdTomato and EGFP from a bidirectional promoter.

FIG. 8 is an image of a western blot showing anti-AAV9 VP1/2/3 (viral proteins 1, 2, 3) immunoreactivity in N. oceanica clones transformed with pIN101, that also expressed tdTomato protein. Western Blot Map: Lane 1—Pageruler Plus Protein Ladder, Lane 2—Wild-Type untransformed N. oceanica, Lane 3—5×10⁹ vector genomes of AAV9. Anti-VP signal is observed at the expected molecular weights of VP1, VP2, and VP3 (81, 66, and 58 kDa, respectively), Lanes 4-12—Individual, genetically unique, transformants of vector pIN101 driving tdTomato and AAV9 VP1 from a bidirectional promoter.

FIG. 9 is an image of a western blot showing anti-AAV9 VP1/2/3 (viral proteins 1, 2, 3) immunoreactivity in N. oceanica clones transformed with pIN102, that also expressed tdTomato protein. Western Blot Map: Lane 1—Pageruler Plus Protein Ladder, Lane 2-Wild-Type untransformed N. oceanica, Lane 3—5×10⁹ vector genomes of AAV9. Anti-VP signal is observed at the expected molecular weights of VP1, VP2, and VP3 (81, 66, and 58 kDa, respectively), Lanes 4-12—Individual, genetically unique, transformants of vector pIN102 driving tdTomato and AAV9 VP2 from a bidirectional promoter.

FIG. 10 is an image of a western blot showing anti-AAV9 VP1/2/3 (viral proteins 1, 2, 3) immunoreactivity in N. oceanica clones transformed with pIN102, that also expressed tdTomato protein. Western Blot Map: Lane 1—Pageruler Plus Protein Ladder, Lane 2-Wild-Type untransformed N. oceanica, Lane 3—5×10⁹ vector genomes of AAV9. Anti-VP signal is observed at the expected molecular weights of VP1, VP2, and VP3 (81, 66, and 58 kDa, respectively), Lanes 4-12—Individual, genetically unique, transformants of vector pIN103 driving tdTomato and AAV9 VP3 from a bidirectional promoter.

FIG. 11 is a schematic showing the generation of a ribosomal DNA (rDNA) locus docking-site with one exemplary expression cassette, IN11, integrated therein. Dotted lines indicate spontaneous homologous recombination between the linearized expression cassette DNA (top) and the N. oceanica chromosome. α-Tub: alpha-tubulin; NeoR: neomycin resistance; Ribosomal Pol I Promoter: RNA Polymerase I promoter; Ribi Prom.: ribosomal subunit bidirectional promoter; UTR: untranslated region; P2A: viral skip peptide P2A.

FIG. 12 are diagrams of expression cassettes used herein to produce AAV9 capsid proteins (IN12, IN16, IN17). All vectors were designed to integrate integrated at the ribosomal docking site. α-Tub: alpha-tubulin; CS: coding sequence; GFP: green fluorescent protein; HygR: hygromycin resistance; LDSP: lipid droplet surface protein; Pol I Prom.: RNA Polymerase I promoter; Ribi Prom.: ribosomal subunit bidirectional promoter; UTR: untranslated region.

FIG. 13 is an image of a Western blot probing for AAV9 VP expression in algae transformed with IN12 and IN17. 1×10⁹ AAV9 capsids were loaded into a lane as a positive control. Equal amounts of late log-phase N. oceanica biomass were loaded in each lane. Multiple uniquely derived clones of IN12 demonstrate stronger anti-VP immunoreactivity at the expected 81 kDa molecular weight when probed using the anti-VP B1 monoclonal antibody. Note that there is a cross-reactive band in the untransformed control (IN11) at ~80 kDa, so a positive signal is interpreted as a stronger and slightly broader band in the 80 kDa range.

FIG. 14 is an image of a Western blot probing for AAV9 VP expression in algae transformed with IN16 and IN17. 1×10⁹ AAV9 capsids were loaded into a lane as a positive control. Equal amounts of late log-phase N. oceanica biomass were loaded in each lane. Multiple uniquely derived clones of IN16 demonstrate strong anti-VP2 immunoreactivity at the expected 66 kDa molecular weight when probed using the anti-VP B1 monoclonal antibody. Similarly, multiple uniquely derived clones of IN17 demonstrate strong anti-VP3 immunoreactivity at the expected 60 kDa. Strong expression of AAV9 VP2 and VP3 are generated from constructs IN16 and IN17, respectively.

FIG. 15 are diagrams of expression vectors used herein to produce AAV9 capsids (IN109, IN17, IN18, IN19, IN21). IN109 is a randomly integrated vector while all others are integrated at the ribosomal docking site. AAP: assembly activating protein; α-Tub: alpha-tubulin; CS: coding sequence; EF1 pom.; elongation factor 1 promoter; GFP: green fluorescent protein; HygR: hygromycin resistance; NeoR: neomycin resistance; LDSP: lipid droplet surface protein; Pol I Prom.: RNA Polymerase I promoter; Ribi Prom.: ribosomal subunit bidirectional promoter; UTR: untranslated region.

FIG. 16 is an image of a Western blot showing a cross comparison of AAV9 VP and AAP ensemble expression strains. SDS-PAGE and Western blotting used the anti-VP B1 monoclonal antibody for detection. Equal amounts of algae biomass grown under the same conditions were loaded per lane. A spike in of 1×10⁹ AAV9 capsids is used as a positive control. Since AAP is required for AAV9 capsid formation, and capsid formation “sequesters” VP monomers that are otherwise degraded by the cell, an increase in VP expression levels (relative to total protein) is expected when capsids are formed. IN21 clearly shows increased VP2 and VP3 expression levels compared to the other candidate AAV producing lines suggesting that it is producing intact capsid.

FIG. 17 is a schematic of an abbreviated workflow for extraction and purification of AAV9 capsids from Nannochloropsis oceanica strain IN21. Frozen cell pellets are lysed by bead-beating in a mild-detergent cocktail. The lysate is vortexed against chloroform (where the majority host proteins and non-capsid AAV VP proteins coagulate and collect at the organic-aqueous interface). AAV9 capsids are then immunoprecipitated using the Poros anti-AAV9 immune-affinity resin that specifically recognizes the five-fold symmetry “peak” of fully formed AAV9 capsids.

FIG. 18 is an image of a dot-blot using anti-ADK9 monoclonal antibody (1:20) that detects intact AAV9 capsids. The control+AAV9 spike in is 5e9 capsids/mL in the initial lysate, or 5e8 capsids total per well equivalent since 100 μL/dot-blot well is loaded. Row A: Total Algae Lysate; Row B: Chloroform Clarified Lysate; Row C: Poros anti-AAV9 Resin “Flow Through”; Row D: Final anti-AAV9 Resin elution. Note that since a phosphatase-based BCIP/NBT chromogenic detection system is used, crude and partially clarified lysate generate very strong non-specific signal because of the endogenous algae host phosphatases are bound to the nitrocellulose. Only Row D, which is highly purified capsid, should be taken as representative of capsid signal.

FIG. 19 is an electron micrograph of a uranyl acetate negative stained Poros anti-AAV9 resin purified fractions. Control algae (IN11) are the original ribosomal locus docking site strain. Mammalian-produced purified AAV9 was spiked into the control strain and purified in the same manner. The producer line is IN21; the ensemble VP1/2/3+AAP cassette integrated into the ribosomal docking site. This strain was grown either at small scale (SS: 12×20 mL cultures in T25 flasks that were pooled together) or at large scale (LS: 500 mL culture in 2 L baffled shake flasks). Arrowheads point to AAV9 capsids. Chevrons point to precipitate or cell debris. For the control condition, microscopy fields with ~30 nm aggregates/precipitates were chosen to show contrast to the highly structured AAV9 capsids. The expected AAV9 capsid size is 25-30 nm. Scale bar=50 nm.

FIG. 20 are drawings of various expression cassettes containing rep coding sequences. AAP: assembly activating protein; α-Tub: alpha-tubulin; β-GS: β-D-glucan synthase; CS: coding sequence; EF1 pom.; elongation factor 1 promoter; NeoR: neomycin resistance; LDSP: lipid droplet surface protein; MCS: multiple coding site; PDH: pyruvate dehydrogenase; PGK: phosphoglycerate kinase; UTR: untranslated region.

### DETAILED DESCRIPTION

The present disclosure relates in part to Nannochloropsis microalgae that produce recombinant virus, methods for making the same, as well as materials, compositions and methods for producing recombinant virus with Nannochloropsis host cells. The recombinant viruses described herein find utility in some embodiments, as gene therapy or vaccine vectors. In preferred embodiments, the recombinant viruses are recombinant adeno-associated viruses.

Certain advantages of the methods and systems provided herein include ease of culturing and maintenance, low production cost, simple purification process, rapid scalability to larger volumes and ability to support large scale viral replication and packaging.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally, the term “about”, as used herein in references to a measurable value such as an amount of weight, time, dose, etc. is meant to encompass values within an acceptable degree of variability in the art. In some embodiments, degree of variability is based on FDA guidelines.

As used herein, “and/or” refers to and encompasses any and all possible combinations of one or more of the associated listed items, as well as the lack of combinations when interpreted in the alternative (“or”).

The term “accessory functions” or “helper functions” refers to non-AAV derived viral and/or cellular functions upon which AAV is dependent for replication. The term includes proteins and RNAs that are required in AAV replication, including moieties involved in activation of AAV gene transcription, stage specific AAV mRNA splicing, AAV DNA replication, synthesis of cap expression products and AAV capsid packaging. Viral-based accessory functions can be derived from any of the known helper viruses such as adenovirus, E1-deleted adenovirus, herpesvirus (other than herpes simplex virus type-1) and vaccinia virus.

A “gene coding sequence” as used herein, refers to a polynucleotide encoding one or more gene products. Gene coding sequences provided herein may or may not include a start codon or stop codon. However, one of ordinary skill in the art will understand that where a coding sequence is provided without a start codon or stop codon, that one can be added.

A “regulatory element”, as used herein, refers to a nucleic acid sequence capable of regulating transcription of a gene (e.g., an AAV capsid gene), and/or regulate the stability or translation of a transcribed mRNA product, and can be present within a transgene. Regulatory elements can comprise at least one transcription factor binding site. Regulatory elements as used herein, in one embodiment, increase or enhance promoter-driven gene expression when compared to the transcription of the gene from the promoter alone in the absence of the regulatory element. Regulatory elements as used herein may occur at any distance (i.e., proximal or distal) to the respective gene coding sequence. Regulatory elements as used herein may comprise part of a larger sequence involved in transcriptional control, e.g., part of a promoter sequence. However, regulatory elements alone are typically not sufficient to initiate transcription on its own and require the presence of a promoter.

A first nucleic acid is “operably linked” to a second nucleic acid when the first nucleic acid is placed into a functional relationship with the second nucleic acid. For example, a promoter or enhancer is operably linked to a coding sequence if it affects the transcription of the sequence; or a ribosome binding site is operably linked to a coding sequence if it is positioned so as to facilitate translation. A first nucleic acid that is “operably linked” to a second nucleic acid need not be directly linked. In other words, there may be intervening sequences between two operably linked nucleic acids.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

“Polypeptide” and “protein” are used interchangeably herein to refer to a polymer of amino acid residues. As used herein, the terms encompass amino acid chains of any length, including full-length proteins, and functional fragments thereof, wherein the amino acid residues are linked by covalent peptide bonds.

A nucleic acid sequence that is “flanked” by two nucleic acid elements indicates that one element is located 5′ to the nucleic acid sequence and the other element is located 3′ to the nucleic acid sequence. A 5′ or 3′ “flanking” element of an expression cassette refers to a nucleic acid sequence is at the farthest 5′ or 3′ end of the expression cassette. The term “flanked” or “flanking” is not intended to indicate that the respective sequences are necessarily contiguous. For example, there may be intervening sequences between a polynucleotide sequence, e.g., a gene coding sequence, and a flanking element, e.g., a homology arm or an inverted terminal repeat (ITR).

As used herein, a first nucleic acid sequence is “upstream” of a second nucleic acid sequence, if the first nucleic acid sequence is located 5′ of the second nucleic acid. As used herein, a first nucleic acid sequence is “downstream” of a second nucleic acid sequence, if the first nucleic acid sequence is located 3′ of the second nucleic acid. A first nucleic acid sequence that is upstream or downstream of a second nucleic acid sequence need not be contiguous with the second nucleic acid sequence.

As used herein, the term “complementary” refers to specific base pairing between nucleotides or nucleic acids. Complementary nucleotides are, generally, A and T (or A and U), and G and C. The polynucleotides and nucleic acids described herein can comprise sequences that are perfectly complementary or substantially complementary (e.g., having a small fraction of mismatched bases) to a genomic sequence. A single stranded nucleic acid for the purposes described herein, also refers to the complementary nucleic acid and a double stranded nucleic acid comprises both strands.

The term “substantial identity” or “substantially identical,” as used in the context of polynucleotide or polypeptide sequences, refers to a sequence that has at least about 60% sequence identity to a reference sequence. Alternatively, percent identity can be any integer from 60% to 100%. Exemplary embodiments include at least about: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215, pp. 403-410 and Altschul et al. (1977). Nucleic Acids Res. 25, pp. 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977). Nucleic Acids Res. 25, pp. 3389-3402). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff (1989). Proc. Natl. Acad. Sci. USA 89, p. 10915).

The BLAST algorithm also performs a statistical analysis of the similarity between two sequences (see, e.g., Karlin and Altschul (1993). Proc. Nat'l. Acad. Sci. USA 90, pp. 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

A “recombinant” substance, such as a recombinant protein or recombinant virus, as used herein, refers to any artificially produced substance, e.g., an artificially produced protein or virus, and is distinguished from naturally produced protein or virus.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month).

As used herein, the terms “packaged” or “encapsidated” refers to the inclusion of a nucleic acid (e.g., a vector genome) in a viral capsid. In embodiments described herein, the viral capsid is an AAV capsid comprising AAV capsid proteins. A vector genome encapsidated by a viral capsid is also referred to herein as an “adeno-associated virus (AAV) particle”. The “AAV capsid” is a near-spherical protein shell that comprises about 60 “AAV capsid proteins” (interchangeably referred to herein as, “AAV capsid protein subunits” or “capsid proteins”) associated and arranged with T=1 icosahedral symmetry. The AAV capsids of the AAV particles described herein comprise a plurality of AAV capsid proteins. When an AAV particle is described as comprising an AAV capsid protein, it will be understood that the AAV particle comprises an AAV capsid, wherein the AAV capsid comprises one or more AAV capsid proteins.

An “expression cassette”, as used herein, refers to a component of DNA comprising at least (i) a promoter sequence, (ii) an open reading frame (e.g., gene coding sequence or a series of gene coding sequences separated by a 2A peptide) and (iii) a 3′ untranslated region (UTR). An expression cassette, in one embodiment, is integrated into a Nannochloropsis host genome. An expression cassette, in one embodiment, is present in a DNA vector, e.g., a plasmid or a portion thereof, prior to it being introduced into the genome of a Nannochloropsis host. An expression cassette, in a further embodiment, comprises a 3′ and 5′ flanking homology arms for site specific genome integration. For the purposes of the present disclosure, the homology arms are considered part of the expression cassette.

An “open reading frame”, as used herein, refers to a coding sequence or a series of gene coding sequences which may be translated into a or a series peptides or proteins. An open reading frame generally contains a start codon (e.g., ATG) at its 5′ end and a stop codon (e.g., TAA, TAG, and TGA) at its 3′ end. Gene coding sequences provided herein may or may not include a start codon or stop codon. However, one of ordinary skill in the art will understand that when an open reading frame contains a series of gene coding sequences separated by a 2A peptide sequence, the gene coding sequence upstream of a 2A peptide sequence does not include a stop codon at its 3′ end; and the gene coding sequence downstream of a 2A peptide sequence does not include a start codon.

The terms “first”, “second”, “third”, “fourth” “fifth”, etc., when used to described a nucleic acid element within an expression cassette, are not intended to imply and do not require a sequential or numerical order of their physical arrangement or presence in the transgene. For example, the present of a first and a third gene or promoter does not require the presence of a second gene or promoter.

As used herein, the term “transgene” refers to an exogenous nucleic acid such as a gene or fragment thereof, artificially introduced into a cell (e.g., into the genome of a cell), or an endogenous gene or portion thereof (e.g., promoter, enhancer, 3′ UTR) artificially introduced into a non-natural locus in the genome of a cell. Transgenes include regions preceding and following a gene coding sequence, regulatory elements, as well as intervening sequences (introns) and flanking sequences such as inverted terminal repeats and/or homology arms. In some embodiments described herein, a transgene includes a nucleic acid sequence that encodes a polypeptide chain or an RNA molecule (e.g., an siRNA). In some embodiments described herein, once integrated into a host genome, an “expression cassette” is referred to as a “transgene”. In some embodiments described herein, a “transgene” is encapsidated by an AAV capsid. Once encapsidated in an AAV capsid, a “transgene” is referred to herein in some embodiments as an “AAV genome” or “vector genome”.

The expression cassettes and transgenes provided herein are mainly described in their broadest sense to indicate the strand of DNA corresponding to the mRNA transcript which is translatable into the protein encoded by the gene coding sequence. However, it should be understood that the expression cassettes and transgenes described herein also encompass non-translated sequences, including the complementary sequence, i.e., the “minus” or “antisense” nucleic acid sequences, as well as double stranded molecules comprising both the plus and minus sequences. “Transgenes” and “expression cassettes” of the disclosure encompass a single stranded molecule comprising a gene coding sequence (i.e., the “plus” or “sense” sequence), a single stranded DNA molecule comprising the antisense or template DNA strand and also, a double stranded version of the transgene or expression cassette. One of ordinary skill will appreciate that with respect to expression cassettes and transgenes comprising bidirectional promoters, the two adjacent genes regulated by the bidirectional promoter will have coding sequences on opposite strands of the double stranded DNA.

“Vector genome” as used herein, is a nucleic acid molecule comprising one or more heterologous nucleic acid sequences. In some embodiments, the one or more heterologous nucleic acid sequences comprises a transgene. In some embodiments, the vector genome comprises at least one inverted terminal repeat (ITR) sequence (e.g., an AAV ITR sequence), optionally two ITRs (e.g., two AAV ITRs), which typically will be at the 5′ and 3′ ends of the vector genome and flank the one or more heterologous nucleic acids (e.g., one or more transgenes). In some embodiments, the vector genome is encapsidated by an AAV capsid to form an AAV particle.

As used herein, the term “wild type” (abbreviated “WT”) refers to the most prevalent form of an organism, strain, gene, protein, or characteristic as it occurs in nature and is distinguished from mutant or variant forms.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As used herein, the terms “introducing” or “delivering” in the context of nucleic acids, for example, expression cassettes, refers to the translocation of the nucleic acid from outside a cell to inside the cell, also referred to sometimes as transformation. Various methods of such translocation are contemplated, including but not limited to, electroporation, contact with nanowires or nanotubes, heat shock, particle bombardment, receptor mediated internalization, translocation via cell penetrating peptides, liposome-mediated translocation, and the like. In one embodiment, the method for transformation of a DNA construct such as an expression cassette into a host cell is chemical transformation, electroporation or particle bombardment. In chemical transformation, cell are made competent (able to take up exogenous DNA) by treatment with divalent cations such as calcium chloride. Heat shock is used to temporarily form pores in the cell membrane, allowing transfer of the exogenous DNA into the cell. In electroporation, a short electrical pulse is used to make the bacterial cell temporarily permeable. Particle bombardment, in one embodiment, comprises the use of gold or tungsten particles coated with the exogenous DNA construct and physically forced into the cell by gene gun.

The present disclosure relates in part to Nannochloropsis microalgae engineered to produce a recombinant virus, e.g., a recombinant AAV (rAAV), and materials and methods for accomplishing the same. The recombinant virus, e.g., rAAV, has utility in some embodiments as a vector for gene therapy or vaccine delivery. The Nannochloropsis microalgae system includes, in one embodiment, a Nannochloropsis host, and at least one expression cassette comprising nucleic acid components for the assembly of a recombinant virus in the Nannochloropsis. In one preferred embodiment, the Nannochloropsis is Nannochloropsis oceanica.

The expression cassettes of the disclosure contain gene coding sequences that when expressed in a Nannochloropsis host, allow for recombinant AAV assembly and production. An “expression cassette” as used herein, may refer to a single expression cassette, or multiple expression cassettes (e.g., a first expression cassette comprising an AAV cap coding sequence, and a second expression cassette comprising an AAV rep coding sequence). The expression cassettes described herein, in one embodiment, are present in a DNA vector such as a DNA plasmid. In another embodiment, the expression cassette of the disclosure is integrated into a Nannochloropsis host genome, either randomly or site-specifically.

The expression cassettes provided herein, in one embodiment, are integrated into a Nannochloropsis host genome to produce a general AAV capsid production strain. ITR-flanked AAV payload vectors can be transformed into these strains to allow for the production of AAV particles comprising an encapsidated genome, e.g., an encapsidated genome comprising a therapeutic transgene.

An expression cassette provided herein can be integrated into a Nannochloropsis host genome so that the cellular machinery of the Nannochloropsis can be harnessed to express the respective gene coding sequences of the expression cassette. Integration into the host genome, in one embodiment, is via homologous recombination. In another embodiment, integration into the host genome is via random integration. In yet another embodiment, integration into the host genome is carried out via the use of a CRISPR associated protein (CAS) in combination with homology-directed repair.

One of ordinary skill in the art will appreciate that if site-specific genome integration into a Nannochloropsis host genome is desired, e.g., via the use of homologous recombination or CRISPR/homology directed repair, that each end of the expression cassette can be flanked by a homology arm to coincide with the genome integration site (i.e., a first homology arm and a second homology arm can be present on the first and second ends of the transgene, and can flank the remaining elements of the transgene). A homology arm, in one embodiment, is from about 100 to about 1000 nucleotides in length, for example, from about 100 to about 900 nucleotides in length, from about 100 to about 800 nucleotides in length, from about 100 to about 700 nucleotides in length, from about from about 100 to about 600 nucleotides in length, or from about 100 to about 500 nucleotides in length. A homology arm, in another embodiment, is from about 200 to about 1000 nucleotides in length, for example, from about 300 to about 1000 nucleotides in length, from about 400 to about 1000 nucleotides in length, from about 500 to about 1000 nucleotides in length, from about 600 to about 1000 nucleotides in length, or from about 700 to about 1000 nucleotides in length. In one embodiment, the homology arm is about 1000 nucleotides in length. In a preferred embodiment, the first and second homology arms are about the same length. The homology arms, in one embodiment, are ribosomal DNA (rDNA) compatible homology arms. In one embodiment, a right and left homology arm comprise the nucleic acid sequence of SEQ ID NO:66 or SEQ ID NO:67, respectively.

In one embodiment, as an alternative to genome integration, gene coding sequences present in an expression cassette of the disclosure can be expressed episomally.

As provided above, reference to an expression cassette or transgene of the disclosure is intended to encompass (i) a single stranded nucleic acid molecule comprising the recited nucleic acid sequence(s), (ii) a double stranded molecule comprising the recited nucleic acid sequence(s) together with the complementary sequence, and/or (iii) a single stranded nucleic acid molecule comprising the complement to the recited nucleic acid sequence(s).

In one aspect of the disclosure, an expression cassette is provided, comprising, (i) a first Nannochloropsis-endogenous promoter comprising a first end and a second end, (ii) a first gene coding sequence operably linked to the first end of the first Nannochloropsis-endogenous promoter, (iii) a first Nannochloropsis-endogenous 3′ untranslated region (UTR) downstream of the first gene coding sequence and operably linked thereto, and (iv) an antibiotic resistance gene coding sequence. The antibiotic resistance gene coding sequence is operably linked to the first Nannochloropsis-endogenous promoter or a second Nannochloropsis-endogenous promoter, and the antibiotic resistance gene coding sequence is operably linked to the first Nannochloropsis-endogenous 3′ UTR or a different Nannochloropsis-endogenous 3′ UTR, and the first gene coding sequence is an adeno-associated virus (AAV) cap coding sequence. In one embodiment, the expression cassette is present in a DNA plasmid prior to integration into a Nannochloropsis host genome.

In one preferred embodiment of an expression cassette comprising an adeno-associated virus (AAV) cap coding sequence, the first Nannochloropsis-endogenous promoter is a bidirectional promoter. In another preferred embodiment of an expression cassette comprising an adeno-associated virus (AAV) cap coding sequence, the Nannochloropsis-endogenous promoter(s) and 3′ UTR(s) provided in an expression cassette of the disclosure are endogenous to Nannochloropsis oceanica.

The first gene coding sequence, in one embodiment, is an AAV cap coding sequence. In a further embodiment, the AAV cap coding sequence is the AAV VP1, VP2 or VP3 coding sequence. In even a further embodiment, the AAV cap coding sequence is the AAV9 VP1, VP2 or VP3 coding sequence. The cap coding sequence in even a further embodiment, comprises the nucleic acid sequence of SEQ ID NO: 31 (AAV9 VP1), 32 (AAV9 VP2), 33 (AAV9 VP2) or 34 (AAV9 VP3).

In one embodiment, an expression cassette of the disclosure comprises a VP1 coding sequence, a VP2 coding sequence and a VP3 coding sequence, each operably linked to different Nannochloropsis-endogenous promoters. In another embodiment, an expression cassette of the disclosure comprises a VP1 gene coding sequence, a VP2 coding sequence and a VP3 coding sequence, where two or more of the VP coding sequences are operably linked to the same Nannochloropsis-endogenous promoter. The Nannochloropsis-endogenous promoter operably linked to a VP coding sequence, in one embodiment, is selected from the Ribi bidirectional promoter, a ribosomal Pol I promoter, and an elongation factor promoter. In yet another embodiment, an expression cassette of the disclosure comprises a VP1 coding sequence, a VP2 coding sequence and a VP3 coding sequence, wherein at least one VP coding sequence is operably linked to a Nannochloropsis-endogenous bidirectional promoter. In a further embodiment, the Nannochloropsis-endogenous bidirectional promoter is the Ribi bidirectional promoter.

An expression cassette of the disclosure, in one embodiment, comprises a bidirectional promoter, two unidirectional promoters, and gene coding sequences for an AAV VP1, VP2, VP3 proteins, in addition to a gene coding sequence for an AAV assembly-activating protein (AAP) protein, each of which is operably linked to one of the aforementioned promoters. In a further embodiment, the expression cassette comprises an antibiotic resistance coding sequence operably linked to one of the aforementioned promoters. In even a further embodiment, the AAV is AAV9 or AAV8. In yet even a further embodiment, the AAV is AAV9.

In another embodiment, an expression cassette comprising a cap coding sequence comprises two bidirectional promoters. In a further embodiment, the expression cassette comprises gene coding sequences for an AAV VP1, VP2, VP3 proteins, in addition to a gene coding sequence for an AAV AAP protein, each of which is operably linked to one of the aforementioned promoters. In a further embodiment, the expression cassette comprises an antibiotic gene coding sequence operably linked to one of the aforementioned promoters. In even a further embodiment, the AAV is AAV9 or AAV8. In yet even a further embodiment, the AAV is AAV9.

In one embodiment described herein, an expression cassette comprising a cap coding sequence comprises a Nannochloropsis-endogenous 5′ UTR downstream of the promoter first end and upstream of the first gene coding sequence, wherein the Nannochloropsis-endogenous 5′ UTR is operably linked to the first gene coding sequence.

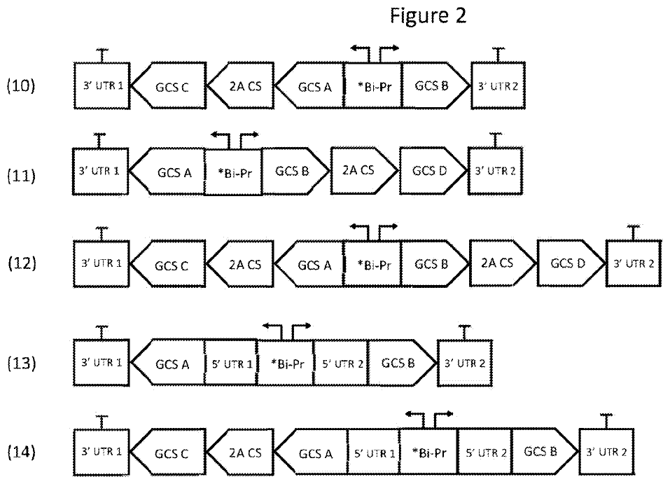

In one embodiment of an expression cassette of the disclosure comprising a cap coding sequence, the first Nannochloropsis-endogenous promoter is a bidirectional promoter. In a further embodiment, the bidirectional promoter drives expression of a gene coding sequence from both the first end and the second end of the promoter. In bidirectional promoter embodiments, it will be appreciated by one of skill in the art that the bidirectional promoter can drive expression of one or more gene coding sequences from each end of the promoter. For example, a 2A peptide coding sequence can be employed on one end of the bidirectional promoter to drive expression of two coding sequences from one or both ends of the promoter. FIGS. 2-5 show exemplary expression cassette embodiments that employ one or more bidirectional promoters. Exemplary bidirectional promoters of the disclosure include but are not limited to the Ribi bidirectional promoter, the VCP2 promoter and the nitrate reductase promoter.

In one embodiment where a 2A peptide coding sequence is employed, the first gene coding sequence is an AAV cap coding sequence, and the second gene coding sequence is an AAV cap coding sequence, an assembly activating protein (AAP) coding sequence, or an antibiotic resistance gene coding sequence. In a further embodiment where a 2A peptide coding sequence is employed, the first gene coding sequence is an AAV VP coding sequence (e.g., VP1, VP2, VP3), and the second gene coding sequence is selected from an AAV VP1, VP2, VP3 coding sequence, an AAP coding sequence, and an antibiotic resistance gene coding sequence, provided that the first coding sequence is different from the second coding sequence. In even a further embodiment, the AAV cap coding sequence is an AAV9 or AAV8 cap coding sequence.

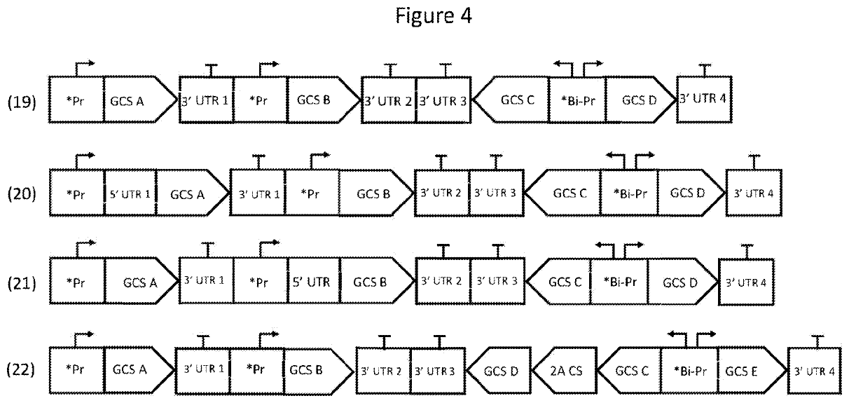

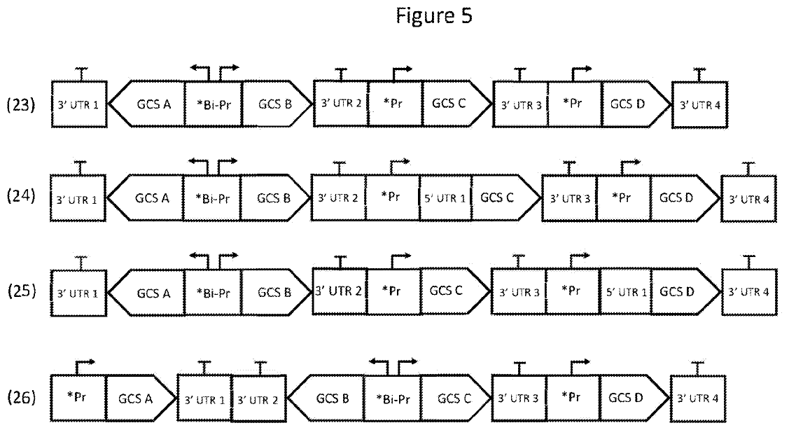

In another embodiment, an expression cassette comprising a cap coding sequence comprises three Nannochloropsis-endogenous promoters, where one of the Nannochloropsis-endogenous promoters is a bidirectional promoter having a first end and a second end, and the other two promoters are unidirectional promoters. See, e.g., FIGS. 4 and 5. In this embodiment, the bidirectional promoter is operably linked to at least one gene coding sequence on each end of the promoter and the 3′ end of each unidirectional promoter is operably linked to at least one gene coding sequence.

In one embodiment of an expression cassette comprising two Nannochloropsis-endogenous promoters, one of the Nannochloropsis-endogenous promoters is a bidirectional promoter having a first end and a second end operably linked to at least one gene coding sequence on each end of the promoter.

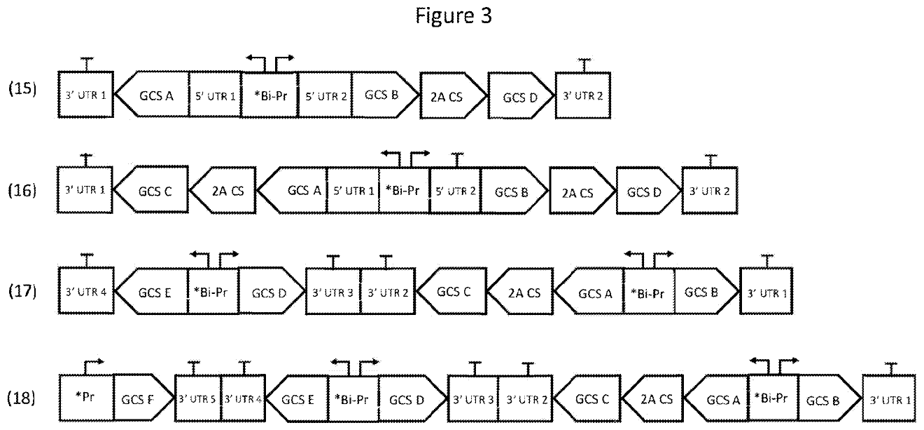

In another embodiment, an expression cassette comprises two Nannochloropsis-endogenous promoters, where each of the promoters is a bidirectional promoter having a first end and a second end. FIG. 3, embodiments 17 and 18, provide examples of such an expression cassette. In a dual bidirectional promoter embodiment, each bidirectional promoter is operably linked to at least one gene coding sequence on each end of the promoter. In even a further embodiment, each bidirectional promoter is operably linked to an AAV rep coding sequence. In a further embodiment, one bidirectional promoter is operably linked to an AAV rep78 coding sequence and the other bidirectional promoter is operably linked to a Rep52 coding sequence. The bidirectional promoters may be constitutively acting, inducible, or a combination thereof.

In another aspect of the disclosure, an expression cassette comprising two Nannochloropsis-endogenous bidirectional promoters is provided. The expression cassette comprises, (i) a constitutively active Nannochloropsis-endogenous bidirectional promoter comprising a first end and a second end, (ii) a first gene coding sequence operably linked to the first end of the constitutively active Nannochloropsis-endogenous bidirectional promoter, wherein the first gene coding sequence is a Rep52 coding sequence, (iii) a first Nannochloropsis-endogenous 3′ untranslated region (UTR) downstream of the first gene coding sequence and operably linked thereto, (iv) a second gene coding sequence operably linked to the second end of the constitutively active Nannochloropsis-endogenous bidirectional promoter, wherein the second gene coding sequence is selected from an AAV assembly activating protein (AAP) gene coding sequence, and an antibiotic resistance gene coding sequence, (v) a second Nannochloropsis-endogenous 3′ untranslated region (UTR) downstream of the second gene coding sequence and operably linked thereto, (vi) an inducible Nannochloropsis-endogenous bidirectional promoter comprising a first end and a second end, (vii) a third gene coding sequence operably linked to the first end of the inducible Nannochloropsis-endogenous bidirectional promoter, wherein the third gene coding sequence is a Rep78 coding sequence, (viii) a third Nannochloropsis-endogenous 3′ untranslated region (UTR) downstream of the third coding sequence and operably linked thereto, (ix) a fourth gene coding sequence operably linked to the second end of the inducible Nannochloropsis-endogenous bidirectional promoter, wherein the fourth gene coding sequence is selected from an adenoviral gene coding sequence and an antibiotic resistance gene coding sequence, and (x) a fourth Nannochloropsis-endogenous 3′ untranslated region (UTR) downstream of the fourth coding sequence and operably linked thereto. The expression cassette comprises a single antibiotic resistance gene coding sequence.

The AAV rep coding sequence, in one embodiment, is a Rep52 or Rep78 coding sequence. The rep gene coding sequence in one embodiment, comprises the nucleic acid sequence of SEQ ID NO:60 (Rep52) or SEQ ID NO:61 (Rep78).

In another embodiment, an expression cassette of the disclosure comprises two bidirectional promoters and two rep coding sequences. In a further embodiment, the expression cassette comprises gene coding sequences for AAV replication proteins Rep78 and Rep52, each of which is operably linked to one of the aforementioned bidirectional promoters. In a further embodiment, the expression cassette comprises an antibiotic gene coding sequence and an adenoviral gene coding sequence selected from a DNA binding protein (DBP) coding sequence and/or a E4orf6 coding sequence operably linked to one of the aforementioned bidirectional promoters. In yet even a further embodiment, the expression cassette comprises an AAV AAP coding sequence, operably linked to one of the bidirectional promoters. Exemplary DBP and E4orf6 coding sequences are provided at SEQ ID NOS: 62 and 63, respectively. An exemplary AAP coding sequence is provided at SEQ ID NO:57.

In one preferred embodiment of an expression cassette comprising a Rep78 coding sequence and a Rep52 coding sequence, the expression of the Rep78 coding sequence is driven by a different bidirectional promoter than the bidirectional promoter driving expression of the Rep52 coding sequence. The Rep78 gene coding sequence, in one embodiment, is operably linked to an inducible promoter, e.g., that is active in the presence of nitrate as the only nitrogen source. One exemplary inducible promoter that can be employed herein is the nitrate reductase promoter. In a further embodiment, the Rep52 gene coding sequence is operably linked to a constitutively active promoter, e.g., the VCP2 bidirectional promoter. In a further embodiment, the bidirectional promoter operably linked on its first end to the Rep78 coding sequence, is operably linked to its second end to an adenoviral gene coding sequence selected from a DNA binding protein (DBP) coding sequence, a E4orf6 coding sequence, or a combination thereof.

An expression cassette of the disclosure, in yet another embodiment, comprises one bidirectional promoter, two unidirectional promoters, and individual gene coding sequences for AAV replication proteins Rep52 and Rep78, each of which is operably linked to one of the aforementioned promoters. Exemplary promoter sequences are provided at SEQ ID NOS: 48 and 49, respectively. In a further embodiment, the expression cassette comprising Rep52 and Rep78 coding sequences comprises an antibiotic gene coding sequence operably linked to one of the aforementioned promoters. In even a further embodiment, the expression cassette comprises an adenoviral gene coding sequence selected from a DNA binding protein (DBP) coding sequence and an E4orf6 coding sequence, or a combination thereof. In yet even a further embodiment, the expression cassette comprises the DNA binding protein (DBP) coding sequence and the E4orf6 coding sequence operably linked to the same promoter. In even a further embodiment, the DBP and E4orf6 coding sequences are operably linked to the same end of a bidirectional promoter, and a 2A peptide coding sequence is present between the two coding sequences. In yet even a further embodiment, the expression cassette comprises an AAV AAP gene coding sequence.

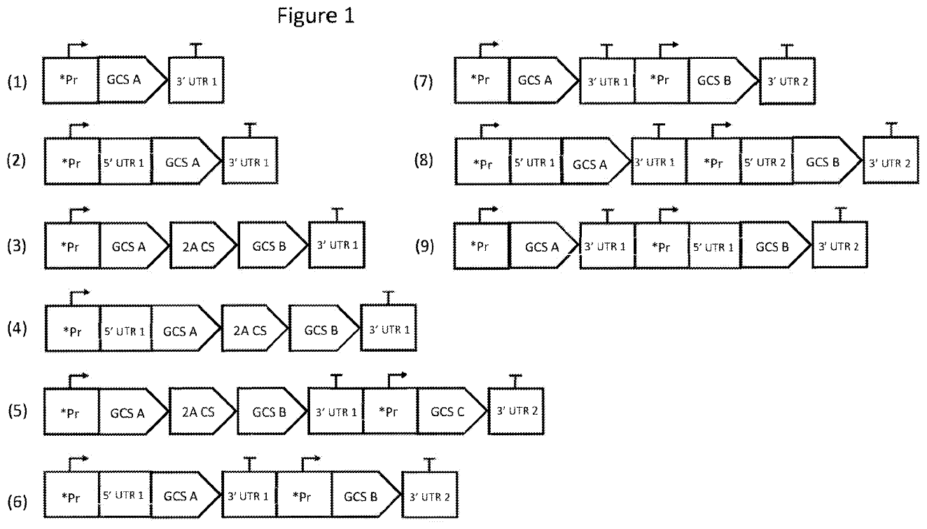

FIGS. 1-5 provide nonlimiting architectures of expression cassettes of the disclosure. It will be appreciated by one of ordinary skill in the art that these expression cassettes and elements thereof can be stacked together to create additional expression cassette architectures.

Embodiments 1-9 in FIG. 1 are exemplary expression cassette architectures, each having one or more Nannochloropsis-endogenous unidirectional promoters operably linked to a gene coding sequence. At least one of the coding sequences in each of these embodiments (abbreviated “GCS” for “gene coding sequence”) is an AAV cap coding sequence or an AAV rep coding sequence. As provided herein, and as shown in FIG. 1 embodiments 3-5, a coding sequence for a 2A peptide can be employed downstream from a first gene coding sequence and upstream of a second coding sequence to facilitate the expression of the first and second coding sequences from one unidirectional promoter. As shown in embodiments 5-9 (FIG. 1), two unidirectional promoters, each operably linked to a different gene coding sequence, are stacked within the expression cassette.

Although not shown explicitly in FIG. 1, one of ordinary skill in the art will appreciate that three, four, five or six unidirectional promoters, each operably linked to a different gene coding sequence, can be stacked within an expression cassette. In embodiments where multiple promoters and gene coding sequences are stacked serially within a single expression cassette, each gene coding sequence is operably linked to a transcription terminator sequence downstream of the gene coding sequence. The transcription terminator sequence in preferred embodiments of the disclosure, is present in a Nannochloropsis-endogenous 3′ UTR.

One of ordinary skill in the art will also appreciate that a bidirectional promoter having a first end and a second end where each end is operably linked to a gene coding sequence can be stacked together with the expression cassette architectures of embodiments 1-9 (FIG. 1).

Embodiments 10-26 in FIGS. 2-5 provide expression cassettes that include a Nannochloropsis-endogenous bidirectional promoter comprising a first end and a second end, operably linked on each end to one or more gene coding sequences. As provided herein, and also shown in FIGS. 2-3 (embodiments 12, 14-18) a 2A peptide coding sequence can be employed downstream from a gene coding sequence to facilitate the expression of two gene coding sequences from one end of a bidirectional promoter.

In one embodiment, an expression cassette of the disclosure includes one or more unidirectional promoters each operably linked to a gene coding sequence and a 3′ UTR, stacked with a bidirectional promoter operably linked to a gene coding sequence on each end. Embodiments 10-16 and 25-26 in FIGS. 3 and 5 encompass such architectures.

In one embodiment of an expression cassette, the Nannochloropsis-endogenous promoter is bidirectional. See, e.g., FIGS. 2-5 for exemplary expression cassette architectures that include bidirectional promoters. In one embodiment where a bidirectional promoter is employed, the bidirectional promoter drives expression of two, three or four gene coding sequences. For example, in one embodiment, the expression cassette comprises a second gene coding sequence operably linked to the second end of the bidirectional promoter, and a second Nannochloropsis-endogenous 3′ UTR operably linked to the second gene coding sequence. In this embodiment, a first and a second gene coding sequence are operably linked to the first end and second end of the bidirectional promoter, respectively. The third gene coding sequence, in one embodiment, encodes one or more viral cap or rep proteins, or antibiotic resistance, and the third gene coding sequence encodes a different protein than the first gene coding sequence and the second gene coding sequence. The third gene coding sequence is located downstream of the first gene coding sequence and upstream of the first 3′ UTR, and is also operably linked to the bidirectional promoter's first end. In another embodiment, a fourth gene coding sequence is provided in an expression cassette, and the fourth gene coding sequence is located downstream of the second gene coding sequence and upstream of the second 3′ UTR, and is also operably linked to the bidirectional promoter's second end.

As shown in FIG. 3, in some embodiments, an expression cassette comprises two Nannochloropsis-endogenous bidirectional promoters, each having a first end and a second end (FIG. 3, embodiments 17 and 18). In one embodiment of such an expression cassette, the first bidirectional promoter is an inducible promoter, e.g., inducible in the presence of nitrate as the lone nitrogen source, and is operably linked on its first end to a Rep78 gene coding sequence. In one embodiment, the inducible promoter is a nitrate reductase promoter. The second bidirectional promoter is constitutively active and is operably linked on its first end to a Rep52 coding sequence. In even a further embodiment, the first bidirectional promoter is operably linked to an adenoviral gene selected from DBP, E4 (e.g., E4orf6), or a combination thereof on its second end. The constitutively active bidirectional promoter, in one embodiment, is the VCP2 promoter. In a further embodiment, the VCP2 promoter comprises a nucleic acid sequence of SEQ ID NO:48. In even a further embodiment, the inducible promoter is a nitrate reductase promoter. In yet a further embodiment, the inducible promoter comprises the nucleic acid sequence of SEQ ID NO:46.

In another embodiment of an expression cassette comprises two Nannochloropsis-endogenous bidirectional promoters, each having a first end and a second end, each promoter is constitutively active. In yet another embodiment of an expression cassette comprising two Nannochloropsis-endogenous bidirectional promoters, each having a first end and a second end, each promoter is an inducible promoter.

Regardless of whether unidirectional promoter, bidirectional promoter or a combination thereof, is employed, all promoters in the expression cassettes provided herein for expressing viral proteins are endogenous to the Nannochloropsis host (sometimes referred to as a packaging cell) in which the expression cassette is delivered. A promoter, present in one embodiment of an expression cassette, is the ubiquitin extension protein (UEP) promoter, a violaxanthin chlorophyll a-binding protein (VCP) promoter, the β-tubulin (β-tub) promoter, the lipid droplet surface protein (LDSP) promoter, the elongation factor (EF) promoter, the ribosomal Pol I promoter, the nitrate reductase promoter, or the ribosomal subunit bidirectional promoter (Ribi). The Nannochloropsis-endogenous promoter in one embodiment, is the lipid droplet surface protein (LDSP) promoter.

In expression cassette embodiments described herein, the nucleic acid components for expression of recombinant virus and viral assembly (e.g., rAAV) may be driven by one or more, two or more, three or more or four or more Nannochloropsis-endogenous promoters. In one preferred embodiment, a transgene includes two Nannochloropsis-endogenous promoters to drive the expression of about four to about six gene coding sequences useful for the assembly of a recombinant virus. In another preferred embodiment, a transgene includes three promoters to drive the expression of about four to about six gene coding sequences useful for the assembly of a recombinant virus. In a further embodiment, the expression cassette comprises AAV VP1, AAV VP2, AAV VP3, AAV AAP coding sequences. In even a further embodiment, the expression cassette comprises an antibiotic resistance gene coding sequence.

An expression cassette of the disclosure in one embodiment, comprises at least one constitutively active promoter. In a further embodiment, all promoters present within an expression cassette are constitutively active.

In some embodiments, a promoter present in an expression cassette of the disclosure is an inducible promoter that can be used to regulate the timing and relative level of gene expression. In one embodiment, the inducible promoter is a unidirectional promoter. In another embodiment, the inducible promoter is a bidirectional promoter. In a further embodiment, the inducible promoter is the nitrate reductase promoter. The nitrate reductase promoter is active when host cells are grown on nitrate as the nitrogen source, and has minimal to no activity when ammonium is the nitrogen source, and nitrate is not present.

An expression cassette of the disclosure in one embodiment, comprises at least one inducible promoter and one constitutively active promoter.

In some embodiments of an expression cassette, the cassette comprises one or more bidirectional promoters. A “bidirectional promoter” is a region of DNA that regulates the expression of two neighboring genes (a first and second gene/gene coding sequence) organized in a head-to-head orientation (5′-5′) on opposite strands of a double stranded DNA molecule. As such, as used herein, a “bidirectional gene pair” or a “bidirectional gene coding sequence pair” is defined as two adjacent genes/gene coding sequences whose coding sequences are located on opposite strands of DNA with transcription start sites (TSSs) not more than about 1 Kilo base pairs (Kb) apart. Bidirectional promoters regulate transcription on both DNA strands and can double the number of transcripts produced from an expression cassette of the disclosure. As provided above, one of ordinary skill will appreciate that with respect to expression cassettes and transgenes comprising bidirectional promoters, the two adjacent genes regulated by the bidirectional promoter will have coding sequences on opposite strands of the double stranded DNA. In one embodiment, the bidirectional promoter is the ribosomal subunit (Ribi) bidirectional promoter, the nitrate reductase (NR) bidirectional promoter, or a violaxanthin chlorophyll a-binding protein (VCP) bidirectional promoter.

In one embodiment, a Nannochloropsis-endogenous bidirectional promoter is operably linked to two or more gene coding sequences utilized for virus particle production and packaging. The bidirectional promoter in one embodiment, is the VCP2 promoter. In another embodiment, the bidirectional promoter is the Ribi promoter. In a further embodiment, the Ribi promoter comprises the nucleic acid sequence set forth in SEQ ID NO:1 or 2. In even a further embodiment, the Ribi promoter comprises the nucleic acid sequence set forth in SEQ ID NO:2.

In a preferred embodiment of an expression cassette comprising an AAV cap coding sequence, the expression cassette comprises a Ribi bidirectional promoter operably linked to a VP coding sequence (e.g., VP3). In even a further embodiment, the expression cassette comprises a Pol I promoter operably linked to a VP1 or VP2 coding sequence and an elongation factor promoter operably linked to a VP1 or VP2 coding sequence, where the Pol I promoter and elongation factor promoters are operably linked to different gene coding sequences. The Ribi bidirectional promoter, in one embodiment, comprises a nucleic acid sequence of SEQ ID NO:1 or SEQ ID NO:2. The AAV in a preferred embodiment, is AAV8 or AAV9.

In yet another embodiment of an expression cassette comprising two Nannochloropsis-endogenous bidirectional promoters, each having a first end and a second end, the first bidirectional promoter is an inducible promoter, e.g., inducible in the presence of nitrate as the lone nitrogen source, and is operably linked on its first end to a Rep78 gene coding sequence. The second bidirectional promoter is constitutively active and is operably linked on its first end to a Rep52 coding sequence. In even a further embodiment, the first bidirectional promoter is operably linked to an adenoviral gene selected from DBP, E40rf6, or a combination thereof on its second end.

In one embodiment, a promoter employed in an expression cassette is from one of the two VCP genes, VCP1 or VCP2 (GenBank: JF946490). See, e.g., Killian et al. (2011). Proc. Natl. Acad. Sci. 108(52), pp. 21265-21269. In a further embodiment, the 3′ untranslated region (3′ UTR) from VCP1 (GenBank: JF957601) or VCP2 is appended to the coding sequence of one or more of the gene coding sequences (transgenes) in the expression cassette.

In another embodiment, the LDSP promoter is present in an expression cassette described herein.

In another embodiment, the elongation factor (EF) promoter is present in an expression cassette described herein.

In yet another embodiment, an expression cassette comprises a VCP promoter.

In one embodiment, an expression cassette provided herein comprises a nitrate reductase (NR) promoter.

In yet even another embodiment, an expression cassette comprises the β-tubulin promoter.

Still in another embodiment, an expression cassette comprises the ribosomal Pol I promoter and the Ribi promoter. In even a further embodiment, the Ribi promoter sequence comprises the nucleic acid sequence of SEQ ID NO:1 or SEQ ID NO:2.

In another embodiment, an expression cassette of the disclosure comprises the chloroplast gene promoter psbA or the nuclear promoter region of the 32 tubulin gene. Other examples of promoters amenable for use herein are hsp70 (“heat shock protein” promoter), ubiquitin extension promoter (UEP), rbcS2 (“rubisco small subunit” promoter), the β-tubulin promoter, and the tubulin α-2 chain (tubA2) promoter.

In one embodiment, the promoter is a Fcp (fucoxanthin chlorophyll-a or -c binding protein) promoter, a nopaline synthase promoter, a nitrate reductase (NR) promoter, or a photosystem I complex protein (PsaD) promoter is present in an expression cassette of the disclosure.

In one embodiment, a VCP promoter is used in an expression cassette described herein.

In another embodiment, the promoter is the ribosomal Pol I promoter (“Pol I promoter”). In a further embodiment, the Pol I promoter comprises the nucleic acid sequence of SEQ ID NO:49.

In one expression cassette embodiment where a Pol I promoter is employed, the Pol I promoter is present in an expression cassette further comprising the Pol I terminator sequence, downstream and operably linked to the gene coding sequence that is operably linked to the Pol I promoter. The Pol I terminator sequence, in one embodiment, is part of a 3′ UTR operably linked to the Pol I promoter.

In one embodiment, an expression cassette for use herein employs a semi-synthetic promoter. The semi-synthetic promoter includes a portion of a promoter endogenous to Nannochloropsis combined with a promoter region from a second promoter, e.g., a second promoter endogenous to Nannochloropsis.

Nannochloropsis oceanica endogenous promoters for use with expression cassettes of the disclosure are also provided in Table 1, below, along with exemplary nucleic acid sequences for the respective promoters.

TABLE 1
Exemplary Nannochloropsis oceanica-endogenous promoters
for use in expression cassettes of the disclosure.

| Nannochloropsis | | |
|---|---|---|
| oceanica-endogenous | Unidirectional or | Exemplary |
| Promoter | Bidirectional? | SEQ ID NO. |
| Ribosomal polymerase I | unidirectional | SEQ ID NO: 49 |
| (Pol I) | | |
| Elongation factor | unidirectional | SEQ ID NO: 50 |
| Ribi | bidirectional | SEQ ID NO: 1 or |
| | | SEQ ID NO: 2 |
| VCP1 | bidirectional | SEQ ID NO: 47 |
| VCP2 | bidirectional | SEQ ID NO: 48 |
| Nitrate reductase | bidirectional | SEQ ID NO: 46 |

TABLE 2
Exemplary Nannochloropsis oceanica-endogenous promoter combinations
for use in expression cassettes of the disclosure.

| Exemplary | | |
|---|---|---|
| embodiment | Promoters | Gene coding sequence |
| Expression | Ribosomal polymerase | VP coding sequence |
| Cassette 1 | I (Pol I) | |
| | Elongation factor | VP coding sequence |
| | Ribi | VP coding sequence, antibiotic |
| | | resistance, AAP coding sequence |
| Expression | Ribosomal polymerase | VP1 coding sequence |
| Cassette 2 | I (Pol I) | |
| | Elongation factor | VP2 coding sequence |
| | Ribi | VP3 coding sequence, antibiotic |
| | | resistance, AAP coding sequence |
| Expression | VCP1 | Rep52 coding sequence, |
| Cassette 3 | | antibiotic resistance |
| | Nitrate reductase | Rep78, adenoviral gene coding |
| | | sequences (e.g., E40rf6, DBP) |
| Expression | VCP2 | Rep52 coding sequence, |
| Cassette 4 | | antibiotic resistance |
| | Nitrate reductase | Rep78, adenoviral gene coding |
| | | sequences (e.g., E40rf6, DBP) |

Each of the expression cassettes provided herein comprises (i) at least one AAV cap coding sequence or (ii) at least one AAV rep gene coding sequence. The present invention is not limited by serotype—in embodiments described herein, the rep and/or cap coding sequence is an AAV1, AAV2, AAV3, AAV4, AAV5, AAV6, AAV7, AAV8, AAV9, AAV10, AAVrh.74, rAAVrh.8, rAAVrh.10, AAV11, AAV12, or AAV13 rep and/or cap coding sequence. In some embodiments, the rep or cap coding sequence encodes a variant rep or cap protein of one of the foregoing AAV serotypes. The variant, in one embodiment, is a deimmunized variant, e.g., the variant has one or more T-cell epitopes or B-cell epitopes depleted compared to a counterpart AAV cap coding sequence.

A “rep gene coding sequence” or “rep coding sequence” as used herein, refers to a gene coding sequence that encodes one or more AAV Rep proteins (Rep78, Rep68, Rep52, and/or Rep40). In the case of AAV, the rep (Replication) gene encodes nonstructural replication proteins Rep78, Rep68, Rep52, and Rep40, that are required for AAV genome replication and packaging. Replication of an AAV genome comprising ITR-flanked DNA requires the longer isoform of the Rep gene product (Rep78 or Rep68) and the shorter Rep52 isoform for packing the ITR-flanked genome into capsids. The Large Rep protein (Rep78/Rep68) processes AAV ITRs to unwind and create necessary nicks used to initiate the replication of AAV genomes (terminal end-resolution). The small Rep isoform (Rep52) physically associates with the capsid and feeds the replicated ITR-flanked DNA into the capsid (see, e.g., Stracker et al. (2004). J. Virol. 78, pp. 441-453; Gonçalves (2005). Virology 2, p. 43, doi: 10.1186/1743-422X-2-43). An exemplary Rep52 coding sequence for use in an expression cassette of the disclosure is provided at SEQ ID NO:60. An exemplary Rep78 coding sequence for use in an expression cassette of the disclosure is provided at SEQ ID NO:61.

In one embodiment, the rep gene coding sequence provided herein encodes a Rep78 protein and/or the Rep52 protein. In one embodiment, two rep gene coding sequences are provided individually in an expression cassette and the two rep gene coding sequences encode a Rep78 protein and the Rep52 protein, respectively. Transcription of Rep78 and Rep52 can be under the control of one promoter (e.g., by using a bidirectional promoter or a 2A peptide coding sequence), or multiple promoters, when present in a single expression cassette. In a preferred embodiment, the Rep78 coding sequence is operably linked to an inducible promoter and Rep52 coding sequence is operably linked to a constitutively active promoter. In even a further embodiment, the Rep78 coding sequence is operably linked to a nitrate reductase promoter and the Rep52 coding sequence is operably linked to a VCP2 promoter.

A “cap coding sequence” or “cap gene coding sequence” as used herein, refers to a gene coding sequence that encodes one or more AAV viral capsid proteins (VP proteins VP1, VP2 and/or VP3).

The AAV cap (Capsid) gene gives rise to the viral capsid proteins (VP; VP1/VP2/VP3) and encodes these proteins in a single open reading frame (ORF). Alternative mRNA splicing and the use of an ATG codon and an alternative start codon for the initiation of VP protein translation lead to appropriate capsid stoichiometry at a VP1/VP2/VP3 ratio of approximately 1:1:10. The VP proteins form the outer capsid shell that protects the viral genome, as well as being involved in cell binding and internalization.

For the purposes of the present disclosure, a rep or cap coding sequence encompasses sequences that give rise to one or multiple gene products (e.g., a single Rep or Cap gene product, or all of the rep or cap gene products).

In another embodiment, the one or more gene coding sequences is an AAV cap coding sequence. In one embodiment, the cap coding sequence is a VP1, VP2 and/or VP3 gene coding sequence. In another embodiment, an expression cassette provided herein includes individual gene coding sequences for each of an AAV VP1, AAV VP2 and AAV VP3. In a further embodiment, the expression cassette provided herein includes individual gene coding sequences for each of an AAV9 VP1, AAV9 VP2 and AAV9 VP3. Transcription of VP1, VP2 and VP3 can be under the control of one promoter (e.g., by using a bidirectional promoter and a 2A peptide), or multiple promoters. In one embodiment, the AAV VP1, VP2 and V3 gene coding sequences are AAV8 VP1, VP2 and V3 gene coding sequences.

In some embodiments, the cap coding sequence encodes an AAV9 VP1 capsid protein, or a variant thereof, for example the protein sequence set forth at SEQ ID NO:25. In a further embodiment, the AAV9 VP1 coding sequence comprises the nucleic acid sequence of SEQ ID NO:31. In some embodiments, the gene coding sequence encodes an AAV9 VP2 capsid protein, or a variant thereof. In one embodiment, the AAV9 VP2 coding sequence encodes amino acid residues 138 to 736 of SEQ ID NO:25. In one embodiment, the AAV9 VP2 capsid protein comprises the amino acid sequence of SEQ ID NO: 26. In some embodiments, the wild type AAV9 VP2 coding sequence comprises the nucleic acid sequence of SEQ ID NO:32 or 33.

In another embodiment, the gene coding sequence encodes the wild type AAV9 VP3 capsid protein, or a variant thereof. In a further embodiment, the wild type AAV9 VP3 coding sequence encodes the amino acid residues 203 to 736 of SEQ ID NO:25. In one embodiment, the wild type AAV9 VP3 capsid protein comprises the amino acid sequence of SEQ ID NO: 27. In some embodiments, the wild type AAV9 VP3 protein is encoded by a gene coding sequence comprising the nucleic acid sequence of SEQ ID NO:34 or 35.

In another embodiment, the gene coding sequence encodes the wild type VP1 AAV8 capsid protein, or variant thereof, which in some embodiments, comprises the amino acid sequence of SEQ ID NO:28. In some embodiments, the gene coding sequence of an expression cassette encodes the wild type AAV8 VP2 capsid protein, which in some embodiments, comprises amino acid residues 138 to 738 of SEQ ID NO:28. In some embodiments, the WT AAV8 VP2 protein encoded by a gene coding sequence comprises the amino acid sequence of SEQ ID NO:29. In some embodiments, the gene coding sequence encodes the wild type AAV8 VP3 capsid protein, or a variant thereof. In a further embodiment, the wild type AAV8 VP3 capsid protein comprises amino acid residues 204 to 738 of SEQ ID NO: 28. In some embodiments, the WT AAV8 VP3 protein encoded by a gene coding sequence of the disclosure comprises the amino acid sequence of SEQ ID NO:30.

As provided throughout, each of the expression cassettes of the disclosure includes at least one rep coding sequence or at least one cap coding sequence, in addition to an antibiotic resistance gene coding sequence. The antibiotic resistance gene coding sequence in one embodiment, comprises a zeocin resistance gene coding sequence (ZeoR gene (shble, GenBank accession number A31898.1)), a neomycin resistance gene coding sequence, a G418 resistance gene coding sequence or a kanamycin resistance gene coding sequence. In another embodiment, the antibiotic resistance gene coding sequence comprises a rifampicin gene coding sequence, a benomyl gene coding sequence, a nystatin gene coding sequence, a spectinomycin gene coding sequence, ampicillin gene coding sequence, or a chloramphenicol gene coding sequence.

In one embodiment, the antibiotic resistance coding sequence present in an expression cassette of the disclosure, is a zeocin, neomycin, G418, kanamycin, rifampicin, benomyl, nystatin, spectinomycin, ampicillin, apramycin, hygromycin B or chloramphenicol resistance coding sequence. In even a further embodiment, the antibiotic resistance coding sequence is a hygromycin B coding sequence.

The antibiotic resistance gene coding sequence can be under the control of the same promoter as the rep gene coding sequence or cap coding sequence, e.g., via the use of a bidirectional promoter or a 2A peptide coding sequence. Table 3 provides exemplary antibiotic resistance gene coding sequences of the disclosure.

TABLE 3

| Antibiotic Resistance | |
|---|---|
| Gene Coding Sequence | Exemplary SEQ ID NO. |
| Neomycin resistance | SEQ ID NO: 51 |
| Hygromycin B resistance | SEQ ID NO: 52 |
| Zeocin resistance | SEQ ID NO: 54 |
| G418 resistance | SEQ ID NO: 55 |
| Blasticidin S resistance | SEQ ID NO: 53 |
| Kanamycin resistance | SEQ ID NO: 56 |

In addition to a rep gene coding sequence or a cap coding sequence, and an antibiotic resistance gene coding sequence, an expression cassette of the disclosure may contain one or more additional gene coding sequences. Along these lines, in one embodiment, an expression cassette of the disclosure comprises a gene coding sequence for the AAV assembly-activating protein (AAP). The AAP is encoded by an alternative ORF present within the cap gene, and the gene product transcription is initiated upstream of the VP3 coding sequence. Sonntag et al. (2010). Proc. Natl. Acad. Sci 107(22), pp. 10220-10225, incorporated by reference herein in its entirety for all purposes. Without wishing to be bound by theory, it is thought that the AAP protein provides a scaffolding function for capsid assembly, and facilitates nucleolar localization of VP proteins. Naso et al. (2017). BioDrugs 31, pp. 317-334. In one embodiment of an expression cassette of the disclosure, a gene coding sequence for the AAP protein, is under the control of the same promoter as a cap or rep gene coding sequence.

Previously, AAV9 capsid formation was shown to be assembly activating protein (AAP) dependent (Grosse et. al. (2017). Journal of Virology 91(20), e01198-17; Earley et al. (2017). Journal of Virology 81(3), e01980-16). AAP is a protein translated from an alternative reading frame found in the AAV cap gene. The start codon of AAP is a non-canonical CTG codon. Genetic analysis of AAV constructs has shown that AAP acts as a capsid formation chaperon and may help shuttle VP proteins from the cytosol to the nucleus and/or nucleolus (the site of AAV capsid formation) (Maurer et al. (2018). Cell Reports 23, pp. 1817-1830). For commonly used AAV serotypes (including AAV1, 2, 6, 7, 8, 9, rh10) AAP must be expressed for successful capsid formation (Earley et al. (2017). Journal of Virology 81(3), e01980-16). The expression of AAP and responding capsid formation efficiency follows a saturable dose-response pattern (Grosse et. al. (2017). Journal of Virology 91(20), e01198-17). In the absence of AAP, the monomeric VP proteins are rapidly degraded and appear to be treated by the host cells as improperly folded proteins which are targeted for proteasomal degradation (Maurer et al. (2018). Cell Reports 23, pp. 1817-1830). Comparatively, AAV serotypes such as AAV4, 5, and 11 can form capsids in an AAP-independent fashion (Earley et al. (2017). Journal of Virology 81(3), e01980-16). Analysis of AAV9 genetics have demonstrated that virus-like-particle capsids can be formed from solely VP3 monomers when AAP is supplied in trans. The transduction efficiency of these VP3-only capsids, however, seems to be diminished with the transduction efficiency being dependent on the presence of VP1 (Sonntag et al. (2017). PNAS 107(22), pp. 10220-10225).

The AAP gene coding sequence, in one embodiment, is an AAV8 or AAV9 AAP coding sequence. The AAP gene coding sequence, in one embodiment, comprises the nucleic acid sequence of SEQ ID NO:57 or 59. The AAP gene coding sequence, in one embodiment, comprises the nucleic acid sequence of SEQ ID NO:76.

The AAP coding sequence, in one embodiment, is under the control of the same promoter as an antibiotic resistance gene coding sequence. The AAP coding sequence, in another embodiment, is under the control of the same promoter as a rep coding sequence or a cap coding sequence.

In even another embodiment of an expression cassette, the one or more gene coding sequences comprises a gene coding sequence for a helper virus function, e.g., to facilitate viral vector replication and/or viral vector assembly within the Nannochloropsis. The helper virus function, in a preferred embodiment, is an AAV helper virus function (e.g., from adenovirus, E1-deleted adenovirus, herpes simplex virus or vaccinia virus). The helper virus function coding sequence in one embodiment, is an E2A, E4 or VA coding sequence. In one embodiment, the helper virus function coding sequence is under the control of the same promoter as a rep coding sequence. In another embodiment, the helper virus function coding sequence is under the control of a different promoter than the promoter operably linked to a rep coding sequence.

In conventional mammalian-cell based AAV production, additional adenoviral-derived gene products are required for efficient ITR-payload replication and overall AAV production. These conventionally include the adenovirus E1, E2A, E4, and VA genes (Smith et al. (2018). Immuno-oncology Insights, doi: 10.18609/cgti.2018.083). In conventional HEK293 cell production of AAVs, the E1 gene is already integrated in the HEK293 host cell line while E2A, E4, and VA are supplied by a helper plasmid (Smith et al. (2018). Immuno-oncology Insights, doi: 10.18609/cgti.2018.083; Lee et al. (2023). Human Gene Therapy 34, pp. 162-170; Matsushita et al. (1998). Gene Ther. 5, pp. 938-945).

Briefly, in the context of HEK293-based AAV production: (1) the E1 gene activates AAV Rep promoters and drives host cells to S-phase where host-polymerases copy the ITR-flanked payload (Matsushita et al. (2004). Journal of General Virology 85, pp. 2209-2214). (2) The E2A gene stimulates AAV genome replication, Rep gene mRNA splicing, and capsid protein production (Carter et al. (1992). Virology 191, pp. 473-476). Adenovirus DNA-binding protein (DBP), encoded in the E2A transcriptional unit, binds to single-stranded ITR-flanked DNA, increases replication processivity, and has been reported to be essential for AAV replication (Stracker et al. (2004). J. Virol. 78, pp. 441-453). (3) The E4 gene (specifically E4orf6) promotes second-strand synthesis of the ITR-flanked payload. E4orf6 inhibits a host cell DNA damage response to double-strand breaks (caused during ITR processing by Rep68 or Rep78) by disrupting the Mre11/Rad50/NBS1 complex (Stracker et al. (2002). Nature 418, pp. 348-352). (4) The adenovirus VA gene encodes multiple RNAs that inhibit the host cell innate immune response protein double-stranded RNA-activated kinase (PKR). During viral infection, general translation is shut-down by a PKR-mediated response. By inhibiting this response, efficient virus protein synthesis is ensured (Mathews and Shenk (1991). J. Virol. 65, pp. 5657-5662; Kitajewski et al. (1986). Cell 45, pp. 195-200).

AAV genome synthesis can be achieved with fewer components than outlined above. In a yeast-based AAV-production system, no adenoviral helper genes were required for ITR-payload replication (Barajas et al. (2017). PLOS One 12: e0173010. Doi: 10.1371/journal.pone.0173010). Instead, simply supplying Rep78 and Rep52 in trans from independent promoters was sufficient for ITR payload packed AAV production. In a mammalian cell-based assay where ITR-flanked DNA replication was the goal and not full AAV production, transduced AAV ITR-flanked DNA was amplified in HEK293 cells by the selective co-expression of E4orf6, DBP, and Rep68 under-inducible promoter expression (Lee et al. (2023). Human Gene Therapy 34, pp. 162-170).

In one embodiment of an expression cassette, a helper virus function coding sequence is under the control of the same promoter as an antibiotic resistance gene coding sequence. In one embodiment of an expression cassette, a helper virus function coding sequence is under the control of a bidirectional promoter.

The helper virus function gene coding sequence, in one embodiment, is present in an expression vector comprising one or more cap coding sequences. In a further embodiment, the helper virus function gene coding sequence is an adenoviral DNA binding protein (DBP) gene coding sequence, an E4orf6 gene coding sequence, or a combination thereof. In even a further embodiment, a DBP gene coding sequence and an E4orf6 gene coding sequence are provided in an expression cassette comprising one or more cap coding sequences. In yet a further embodiment, the DBP gene coding sequence and the E4orf6 gene coding sequence are under the control of the same promoter, e.g., via the use of an intervening 2A peptide coding sequence. In yet a further embodiment of an expression cassette, the DBP gene coding sequence and the E4orf6 gene coding sequence are under the control of a bidirectional promoter.

The helper virus function gene coding sequence, in one embodiment, is present in an expression vector comprising one or more rep gene coding sequences. In a further embodiment, the helper virus function gene coding sequence is an adenoviral DNA binding protein (DBP) gene coding sequence, an E4orf6 gene coding sequence, or a combination thereof. In even a further embodiment, a DBP gene coding sequence and an E4orf6 gene coding sequence are provided in an expression cassette comprising one or more cap coding sequences. In yet a further embodiment, the DBP gene coding sequence and the E4orf6 gene coding sequence are under the control of the same promoter, e.g., via the use of an intervening 2A peptide coding sequence. In even yet a further embodiment of an expression cassette, the DBP gene coding sequence and the E4orf6 gene coding sequence are under the control of a bidirectional promoter.

Other helper virus function gene coding sequences may also be included in an expression cassette of the disclosure. For example, an adenoviral factor E1, E2A, E4, and VA coding sequence, or a combination thereof, may be included in an expression cassette. This can be accomplished through the use of additional 2A peptides coding sequences downstream of other helper gene, cap, rep, AAP or antibiotic resistance coding sequences. Alternatively or additionally, the helper virus function gene can be included in an expression vector by operably linking to an additional promoter and 3′ UTR.

In the expression cassettes described herein, transcription of multiple genes can be driven from the same promoter, i.e., a Nannochloropsis-endogenous promoter, which in a preferred embodiment is Nannochloropsis oceanica promoter. This can be accomplished for example, via the use of a bidirectional promoter, discussed above, and/or by inserting a nucleic acid encoding a viral 2A peptide (herein “2A” or “2A peptide”) downstream of a first gene coding sequence and upstream of a second gene coding sequence, where both the first and second gene coding sequences are operably linked to the same promoter. The 2A peptide facilitates polycistronic gene expression of two or more gene coding sequences of an expression cassette, for example, two or more of the VP coding sequences, rep coding sequences, AAP coding sequence, helper function coding sequence, antibiotic resistance coding sequence, or a combination of any of the foregoing.

2A peptide coding sequences, in embodiments described herein, may be utilized downstream of any gene coding sequence to allow for expression of multiple coding sequences from a single promoter end. An expression cassette, in one embodiment, comprises a 2A peptide coding sequence downstream of the first gene coding sequence, and a second gene coding sequence downstream of the 2A peptide coding sequence and upstream of the 3′ UTR, and operably linked to the first end of the Nannochloropsis-endogenous promoter. In such embodiments, the Nannochloropsis-endogenous promoter drives expression of both the first gene coding sequence and the second gene coding sequence.

In one embodiment of an expression cassette of the disclosure, a nucleic acid encoding a 2A peptide is provided between a first and a second gene coding sequence that are operably linked to the same end of the same promoter. As such, in some embodiments, the nucleic acid encoding a 2A peptide is operably linked to the 3′ end of the first coding sequence and the 5′ end of the second coding sequence. One of ordinary skill in the art will appreciate that where a nucleic acid encoding a 2A peptide is used in combination with a unidirectional promoter in an expression cassette, the expression of a minimum of two gene coding sequence can be driven from the unidirectional promoter. Similarly, where a bidirectional promoter is employed together with a 2A peptide coding sequence, the expression of at least three gene coding sequences can be driven from the single bidirectional promoter. See, e.g., FIGS. 1-5 for various expression cassette architectures amenable for use herein that employ 2A peptide coding sequences.

In another embodiment, the expression cassette further comprises a nucleic acid encoding a 2A peptide downstream of the first gene coding sequence (labeled ‘GCS A’ in FIG. 1), and a second gene coding sequence (also referred to as a transgene, labeled ‘GCS B’ in FIG. 1) immediately downstream of the 2A peptide and immediately upstream of the 3′ UTR. See, e.g., FIG. 1, embodiments 3 and 4. In one embodiment, the second gene coding sequence (labeled ‘GCS B’ in FIG. 1) encodes a rep or a cap protein or AAP. In another embodiment, the second gene coding (labeled ‘GCS B’ in FIG. 1) sequence encodes an antibiotic resistance gene.

In another embodiment of an expression cassette comprising a bidirectional promoter, the expression cassette further comprises a nucleic acid encoding a 2A peptide immediately downstream of the third gene coding sequence (labeled ‘GCS B’ in FIG. 2), and operably linked thereto. A fourth gene coding sequence (labeled ‘GCS D’ in FIG. 2) is immediately downstream of the second 2A peptide coding sequence and immediately upstream of the second 3′ UTR. FIG. 2, embodiment 12 shows one example of this architecture.

2A peptides are characterized by a C-terminal ‘-D(V/I)ExNPGP-’ (SEQ ID NO:14) motif where ‘x’ can be any amino acid, that have been reported to (i) pause ribosomes (sometimes referred to as “ribosomal skipping”) and (ii) drive a translational recoding event, in which two separate proteins from an ORF containing a 2A peptide coding sequence (the ‘upstream’ and ‘downstream’ products) are produced. A ‘break’ occurs in the polypeptide backbone between the final 2 amino acids of the 2A peptide, Gly and Pro. The ‘break’ results in Pro being the first amino acid of the downstream protein. See, e.g., Sharma et al. (2012). Nuc. Acids Res. 40(7), pp. 3143-3151, incorporated by reference herein in its entirety.

The nucleic acid encoding a 2A peptide, in one embodiment, is from about 19 amino acids to about 65 amino acids in length. In one embodiment, the 2A peptide coding sequence encodes the 2A peptide T2A (SEQ ID NO: 3), F2A (SEQ ID NO: 4), or P2A (SEQ ID NO: 5). In one embodiment, the 2A peptide comprises a C-terminal domain comprising T2A (SEQ ID NO: 3), F2A (SEQ ID NO: 4), or P2A (SEQ ID NO: 5). In one embodiment, the 2A peptide consists of the T2A (SEQ ID NO: 3), F2A (SEQ ID NO: 4), or P2A (SEQ ID NO: 5) peptide. Nucleic acids encoding any of the foregoing can be used in expression cassettes provided herein.

In one embodiment, a gene coding sequence encoding the 2A peptide from the foot-and-mouth disease virus (FMDV) is used in an expression cassette of the disclosure.

In one embodiment, the 2A peptide encoded by a gene coding sequence of an expression cassette of the disclosure, comprises a sequence set forth in SEQ ID NO: 10. In another embodiment, the 2A peptide consists of the amino acid sequence set forth in SEQ ID NO: 10. In yet another embodiment, the 2A peptide comprises a C-terminal domain comprising SEQ ID NO:10. In one embodiment, the 2A peptide is encoded by the nucleic acid sequence set forth at SEQ ID NO:11-13.

In one embodiment, the 2A peptide encoded by a gene coding sequence of an expression cassette of the disclosure, comprises the T2A peptide sequence (SEQ ID NO: 3) as a C-terminal domain. In one embodiment, the 2A peptide comprises the F2A peptide as a C-terminal domain (SEQ ID NO: 4). In one embodiment, the 2A peptide comprises the P2A peptide as a C-terminal domain (SEQ ID NO: 5). Nucleic acids encoding any of the foregoing can be used in expression cassettes provided herein.

In one embodiment, the 2A peptide comprises an amino acid sequence set forth in one of SEQ ID NO: 3-10. Nucleic acids encoding any of the foregoing can be used in expression cassettes provided herein.

In another embodiment, the 2A peptide comprises an amino acid sequence set forth in SEQ ID NO: 6, 7, 8, 9 or 10. Nucleic acids encoding any of the foregoing can be used in expression cassettes provided herein.

In yet another embodiment, the 2A peptide encoded by an expression cassette consists of the amino acid sequence set forth in SEQ ID NO: 6, 7, 8, 9 or 10. In even another embodiment, the 2A peptide comprises a C-terminal domain corresponding to an amino acid sequence set forth in SEQ ID NO: 6, 7, 8, 9 or 10. Nucleic acids encoding any of the foregoing can be used in expression cassettes provided herein.

The expression cassettes of the disclosure include a Nannochloropsis-endogenous 3′ untranslated region (UTR) downstream of a gene coding sequence (e.g., a rep or cap coding sequence, antibiotic resistance coding sequence, AAP coding sequence, etc.) and is operably linked thereto. Without wishing to be bound by theory, the 3′ UTR is thought to facilitate efficient and complete gene expression.

A 3′ UTR, in a preferred embodiment, comprises a transcription termination sequence.

In one embodiment of the transgene, the Nannochloropsis 3′ UTR operably linked to the third gene coding sequence is the VCP1 3′ UTR, VCP2 3′ UTR, the LDSP 3′ UTR, the heat shock protein 3′ UTR, or the cellulose synthase (CS) 3′ UTR. In a further embodiment, the 3′ UTR is the LDSP 3′ UTR. In even a further embodiment, the LDSP 3′ UTR comprises the nucleic acid sequence of SEQ ID NO:36.

In one embodiment, the 3′ UTR is the VCP1 3′ UTR (GenBank: JF957601). In another embodiment, the 3′ UTR is the VCP2 3′ UTR.

In another embodiment, the 3′ UTR is the LDSP 3′ UTR. In one embodiment, the LDSP 3′ UTR comprises the nucleic acid sequence of SEQ ID NO: 36 or 80. In yet another embodiment, the 3′ UTR is the heat shock protein 3′ UTR. In still even another embodiment, the 3′ UTR is the cellulose synthase (CS) 3′ UTR. In even a further embodiment, the CS 3′ UTR comprises the nucleic acid sequence of SEQ ID NO:39.

In another embodiment, the 3′ UTR is the LDSP 3′ UTR. In yet another embodiment, the 3′ UTR is the Dox9 3′ UTR. In even a further embodiment, the Dox9 3′ UTR comprises the nucleic acid sequence of SEQ ID NO:40.

In yet another embodiment, a redoxin 3′ UTR is present in an expression vector. In a further embodiment, the redoxin 3′ UTR is downstream of a rep gene coding sequence. In even a further embodiment, the rep gene coding sequence is the Rep78 gene coding sequence. In one embodiment, the redoxin 3′ UTR comprises the nucleic acid sequence of SEQ ID NO:42.

In even yet embodiment, a phosphoglycerate kinase (PGK) 3′ UTR is present in an expression vector. In a further embodiment, the PGK 3′ UTR is downstream of a rep gene coding sequence. In even a further embodiment, the rep gene coding sequence is the Rep52 gene coding sequence. In one embodiment, the PGK 3′ UTR comprises the nucleic acid sequence of SEQ ID NO:43.

In yet another embodiment, a pyruvate dehydrogenase (PDH) 3′ UTR is present in an expression vector. In a further embodiment, the PDH 3′ UTR is downstream of a cap coding sequence or an AAP coding sequence and operably linked thereto. In even a further embodiment, the PDH 3′ UTR is downstream of an AAP coding sequence and operably linked thereto. In yet even a further embodiment, the expression cassette comprises a rep gene coding sequence operably linked to a different promoter than the promoter driving expression of the AAP coding sequence. In one embodiment, the PDH 3′ UTR comprises the nucleic acid sequence of SEQ ID NO:44.

In yet another embodiment, an expression cassette comprises an alpha-tubulin 3′ UTR. In one embodiment, the alpha-tubulin 3′ UTR comprises the nucleic acid sequence of SEQ ID NO:37 or 38.

In yet even another embodiment, an expression cassette comprises a 1,3-β-D-glucan synthase (β-GS) 3′UTR. In a further embodiment, the β-GS 3′ UTR is downstream of a viral helper function gene coding sequence. In even a further embodiment, the viral helper function gene coding sequence is the DBP and/or E4orf6 coding sequence. In one embodiment, the β-GS 3′ UTR comprises the nucleic acid sequence of SEQ ID NO:45.

The 3′ UTRs and promoters used in the expression cassettes of the disclosure are endogenous to the Nannochloropsis host that the respective expression cassette will be introduced to. In a preferred embodiment, the Nannochloropsis is Nannochloropsis oceanica. In one embodiment, a promoter is derived from the same Nannochloropsis gene as the 3′ UTR that the promoter is operably linked to. In another embodiment, an operably linked promoter and 3′ UTR are each derived from different Nannochloropsis genes.

In one embodiment, an expression cassette provided herein further comprises a 5′ UTR. The 5′ UTR, in one embodiment, is endogenous to the Nannochloropsis host and is upstream of a gene coding sequence and operably linked thereto. The 5′ UTR, in one embodiment, is endogenous to the Nannochloropsis host and can be derived from the same or a different gene than a 3′ UTR that are operably linked to a gene coding sequence. In one embodiment, the 5′ UTR comprises a consensus Kozak sequence (see, e.g., SEQ ID NOS: 81-84) or a leader-enhancing sequence, to, for example enhance translational efficiency of virus particle components. See, e.g., Poliner et al. (2018). Plant Cell Reports 37, pp. 1383-1399, the content of which is incorporated by reference in its entirety for all purposes.

In one embodiment, the promoter and 5′ UTR downstream of the promoter, and operably linked thereto, in an expression cassette of the disclosure, are both endogenous to the Nannochloropsis host and derived from the same gene. In another embodiment, the promoter and 5′ UTR operably linked to the promoter are both endogenous to the Nannochloropsis host and derived from different genes.

In one embodiment, the expression cassette comprises both a 5′ UTR and 3′ UTR and both are endogenous to the Nannochloropsis host. In a further embodiment, the 5′ UTR and 3′ UTR are derived from different Nannochloropsis genes. In one embodiment, the promoter and 5′ UTR in an expression cassette are derived from the same Nannochloropsis gene.

In expression cassette embodiments where a bidirectional promoter is employed, the expression cassette can comprise a 5′ UTR downstream of one or both of the promoter's first end and second end. In embodiments where a 5′ UTR is operably linked to both the first and second end of the bidirectional promoter, and the 5′ UTR used may be the same or different. Additionally or alternatively, the 5′ UTR used with a bidirectional promoter may be derived from the same or different gene as the 3′ UTR that is operably linked to the same end of the bidirectional promoter as the 5′ UTR.

An expression cassette provided herein, in one embodiment, further comprises a cellular localization sequence to drive the localization of a protein encoded by the expression cassette to a particular cellular compartment. In one embodiment, an N-terminal targeting sequence is appended to the gene coding sequence, e.g., a gene encoding a viral assembly protein. In one embodiment, the targeting sequence is an endoplasmic reticulum (ER) targeting sequence. In a further embodiment, the targeting sequence is a nucleic acid encoding amino acids 1-28 of the protein disulphide isomerase (PDI) (NCBI protein ID: AAA34848.1). In one embodiment, the targeting sequence is a mitochondria targeting sequence. In a further embodiment, the targeting sequence is a nucleic acid encoding amino acids 1-104 of the oxidase assembly protein 1 (Oxa1) (NCBI protein ID: CAA54675.1). In another embodiment, the targeting sequence is a periplastidal compartment (PPC) targeting sequence. In a further embodiment, the targeting sequence is a nucleic acid encoding amino acids 1-88 of the symbiotic Der1-1 (sDer1-1) (NCBI protein ID: XP 002176640.1). In even another embodiment, the targeting sequence is a stroma targeting sequence. In a further embodiment, the targeting sequence is a nucleic acid encoding amino acids 1-33 of the violaxanthin/chlorophyll a-binding protein 1 (VCP1) (NCBI protein ID: AET85054.1).

In yet another embodiment, a nuclear localization sequence is appended to the rep, cap or AAP coding sequence. The nuclear localization sequence, in one embodiment, is appended to the 5′ end of the transgene encoding a protein required for viral assembly and packaging. In another embodiment, the nuclear localization sequence is appended to the 3′ end of the transgene encoding a protein required for viral assembly and packaging. In one preferred embodiment, the nuclear localization sequence is the SV40 nuclear localization sequence, which codes for the peptide Pro-Lys-Lys-Lys-Arg-Lys-Val (SEQ ID NO:11). Other nuclear localization sequences known in the art can be employed herein, for example, one of the nuclear localization sequences described in Lu et al. (2021). Cell Commun. Signal 19(60) can be employed. Lu et al. (2021). Cell Commun. Signal 19(60) is incorporated by reference herein in its entirety for all purposes.

In some embodiments, one or more elements of an expression cassette is codon-optimized for expression in the Nannochloropsis microalgae. In a further embodiment, the Nannochloropsis is Nannochloropsis oceanica.

The expression cassette, in one embodiment, comprises an intron upstream or downstream of the gene coding sequence, i.e., the coding sequence for a viral assembly protein or antibiotic resistance. The intron, in one embodiment, enhances transgene expression and/or stability of the expressed RNA transcript. In some embodiments, the intron is downstream (i.e., 3′) of the promoter and upstream (i.e., 5′) of the transgene coding sequence. In another embodiment, the intron is downstream (i.e., 3′) of the transgene. In even another embodiment, the intron is present within the coding sequence of a viral assembly protein, e.g., a viral rep or cap protein.

In one embodiment of an AAV particle produced by a Nannochloropsis host, the AAV particle comprises an encapsidated genome. An AAV genome, once encapsidated, can be referred to as a “vector genome”. As such, in one embodiment, an expression cassette of the disclosure further comprises an AAV genome cassette, i.e., for encapsidation by the AAV particle. The AAV genome, prior to encapsidation, is delivered to a Nannochloropsis in an AAV genome cassette. The AAV genome cassette, in one embodiment, is appended to one of the expression cassettes provided herein, to provide a “superstacked” cassette (e.g., appended to an expression cassette comprising one or more rep coding sequences). The AAV genome cassette (components discussed below), contain at a minimum, a promoter, a therapeutic gene coding sequence and a poly(A) signal, where these components are flanked by a 3′ ITR and a 5′ ITR. The AAV genome cassette, in this embodiment, is upstream or downstream of the expression cassette comprising either the rep or cap coding sequence. Components of AAV genome cassettes (referred to as “vector genomes” once encapsidated), are discussed below.

CRISPR-mediated, homology-directed integration into a known locus can be used to deliver the ITR-flanked AAV genome cassette. For example, if site specific genome integration is desired, then homology arms can be provided that flank the superstacked cassette, i.e., one homology arm is upstream of the AAV genome cassette and the second homology arm is downstream of the expression cassette comprising either the rep or cap coding sequence.

Alternatively, if site specific genome integration is desired at a different site than where integration of the rep or cap cassette occurs, then the AAV genome cassette in one embodiment, is flanked with appropriate homology arms that differ from the homology arms of the other expression cassettes. In the case of homology-directed integration of one embodiment of an AAV genome cassette, one such integration locus is found between the 3′ UTRs of a MutS mismatch repair gene (transcript estExt_Genewise1.C_170242) and a HSP70 chaperon gene (transcript CE116060_6174). This site represents an open-chromatin locus for locating an ITR-flanked payload because two constitutively transcribed “housekeeping” genes are located on each side of the locus. In addition, the site between the “facing-in” 3′-UTRs is non-coding and unlikely regulatory.

The AAV genome, in one embodiment, can be provided in a DNA plasmid comprising one of the expression cassettes described herein, e.g., via the use of a plasmid containing multiple cloning sites. In the case of transient transformation, an AAV vector genome cassette can be inserted into a DNA plasmid and constructed, for example, according to the method of Karas et al. (2015). Nat. Communications 6:1925, or Poliner et al. (2019). Plant J. 99(1): 112-127, the disclosure of each of which is incorporated by reference herein in its entirety for all purposes.

In another embodiment, the ITR-flanked AAV genome payload cassette can be electroporated into a Nannochloropsis host comprising a rep and/or cap Rep/Cap expressing algae as a circular plasmid containing the yeast CEN and ARS sequences that allow for autonomous replication of episomal DNA (plasmids) in N. oceanica. See Poliner et al. (2018). ACS Synth. Biol. 7, pp. 962-968.

The identity and architectures of AAV genomes (and expression cassettes comprising the same) will vary depending on the type of virus being produced and its ultimate use, e.g., in gene therapy. Vector genome components encapsidated by a recombinant AAV are discussed in detail below.

In some embodiments, the vector genome is a recombinant AAV (rAAV) genome. The rAAV genome, in one embodiment, comprises a 5′ AAV ITR and a 3′ AAV ITR flanking a heterologous polynucleotide of interest, e.g., a therapeutic transgene. If the polynucleotide of interest is to be transcribed it is operatively linked to transcriptional control DNA, specifically promoter DNA and polyadenylation signal sequence DNA that are functional in target cells to form an AAV genome cassette. The vector genome, as discussed in further detail below, may also include intron sequences to facilitate processing of an RNA transcript when expressed in mammalian cells. In another embodiment, the transgene in the rAAV vector genome is an inhibitor RNA. The inhibitor RNAs may be antisense RNAs, ribozymes, small interfering RNAs (RNAi), miRNA or aptamers. Commercial providers such as Ambion Inc. (Austin, Tex.), Darmacon Inc. (Lafayette, Colo.), InvivoGen (San Diego, Calif.), and Molecular Research Laboratories, LLC (Herndon, Va.) generate custom siRNA molecules. In addition, commercial kits are available to produce custom siRNA molecules, such as SILENCER™ siRNA Construction Kit (Ambion Inc., Austin, Tex.) or psiRNA System (InvivoGen, San Diego, Calif.).

The vector genomes of the disclosure lack AAV rep and cap DNA. These are provided in trans to a Nannochloropsis host via one or more of the expression cassettes of the disclosure. AAV DNA in the rAAV genomes may be from any AAV serotype for which a recombinant virus can be derived including, but not limited to, AAV serotypes AAVrh.8, AAVrh.10, AAVrh.74, AAV1, AAV2, AAV3, AAV4, AAV5, AAV6, AAV7, AAV8, AAV9, AAV10, AAV11, AAV12, and AAV13. The nucleotide sequences of the genomes of the AAV serotypes are known in the art. For example, the complete genome of AAV1 is provided in GenBank Accession No. NC_002077; the complete genome of AAV2 is provided in GenBank Accession No. NC_001401 and Srivastava et al., J. Virol., 45:555-564 11983, incorporated by reference herein in its entirety for all purposes); the complete genome of AAV3 is provided in GenBank Accession No. NC_1829, incorporated by reference herein in its entirety for all purposes; the complete genome of AAV4 is provided in GenBank Accession No. NC_001829, incorporated by reference herein in its entirety for all purposes; the AAV5 genome is provided in GenBank Accession No. AF085716, incorporated by reference herein in its entirety for all purposes; the complete genome of AAV6 is provided in GenBank Accession No. NC_00 1862, incorporated by reference herein in its entirety for all purposes; at least portions of AAV7 and AAV8 genomes are provided in GenBank Accession Nos. AX753246 and AX753249, respectively, incorporated by reference herein in its entirety for all purposes; the AAV9 genome is provided in Gao et al., J. Virol., 78:6381-6388 (2004), incorporated by reference herein in its entirety for all purposes; the AAV10 genome is provided in Mol. Ther., 13 (1): 67-76 (2006), incorporated by reference herein in its entirety for all purposes; the AAV11 genome is provided in Virology, 330(2): 375-383 (2004), incorporated by reference herein in its entirety for all purposes, the genome of AAV12 is provided in GenBank Accession No. DQ813647.1, incorporated by reference herein in its entirety for all purposes, and the genome of AAV13 is provided in GenBank Accession No. EU285562.1, incorporated by reference herein in its entirety for all purposes.

AAV genomes described herein can include one or more regulatory elements, for example one or more regulatory elements upstream of the gene coding sequence (e.g., the therapeutic gene coding sequence). The encapsidated genome (also referred to as a vector genome), in one embodiment, comprises from 5′ to 3′, a 5′ ITR, a promoter, a transgene, a poly(A) signal; and a 3′ ITR. In another embodiment, the encapsidated vector genome comprises from 5′ to 3′, a 5′ ITR, an enhancer, a promoter, a transgene, a poly(A) tail; and a 3′ ITR. The encapsidated genome, in even another embodiment, comprises from 5′ to 3′, a 5′ ITR, a promoter, a SV40 intron, a transgene, a poly(A) tail; and a 3′ ITR. In yet even another embodiment, the encapsidated genome comprises from 5′ to 3′, a 5′ ITR, an enhancer, a promoter, an intron, a transgene, a poly(A) tail; and a 3′ ITR. Other vector genome architectures are also discussed herein. Components of vector genomes for use in the present disclosure are discussed below.

Inverted terminal repeats (ITR) are palindromic 145 nucleotide sequences that flank a transgene. The 5′ and 3′ ITRs of an AAV vector genome are necessary for both the integration of the transgene into the host cell genome (e.g., chromosome 19 in humans) and for encapsidation of the transgene into the AAV particle.

In some embodiments, AAV vector genomes of the present disclosure comprise ITR sequences from any one AAV serotype, for example, AAVrh.74, AAV1, AAV2, AAV3, AAV4, AAV5, AAV6, AAV8, rAAVrh.8, rAAVrh.10, AAV9, AAV10, AAV11, AAV12, or AAV13. In some embodiments, the AAV vector genomes disclosed herein comprise a 5′ AAV2 ITR and a 3′ AAV2 ITR sequence.

In some embodiments, AAV vector genomes described herein comprise a 5′ AAV2 ITR having at least 80%, 85%, 90%, 91%, 92%, 93%, 94%, 95%, 96%, 97%, 98%, 99%, or 100% sequence identity to SEQ ID NO: 16. In some embodiments, the 5′ AAV2 ITR comprises SEQ ID NO: 16. In some embodiments, the 5′ AAV2 ITR consists of SEQ ID NO:16.

In some embodiments, AAV vector genomes described herein comprise a 3′ AAV2 ITR having at least 80%, 85%, 90%, 91%, 92%, 93%, 94%, 95%, 96%, 97%, 98%, 99%, or 100% sequence identity to SEQ ID NO: 17. In some embodiments, the 3′ AAV2 ITR comprises SEQ ID NO: 17. In some embodiments, the 3′ AAV2 ITR consists of SEQ ID NO: 17.

Promoters drive the expression of a polynucleotide of interest (e.g., therapeutic gene) (and are typically located upstream (or 5′) of the polynucleotide whose expression they regulate.

In some embodiments, vector genomes of the present disclosure comprise a mammalian promoter, for example, human, non-human primate (e.g., cynomolgous macaque), mouse, horse, cow, pig, cat, and dog promoters. In some embodiments, recombinant AAV vector genomes disclosed herein comprise strong, constitutively active promoters to drive high-level expression of the transgene. For example, in some embodiments, the promoter is a cytomegalovirus (CMV) promoter/enhancer, an elongation factor 1α (EF1α) promoter, a simian virus 40 (SV40) promoter, a chicken β-actin hybrid promoter, or a CAG promoter.

In one embodiment, the promoter is a thyroxine binding globulin (TGB) promoter, a transthyretin (TTR) promoter, or a chicken β-actin promoter.

The promoter, in one embodiment, is an MHCK7 or chicken β-actin hybrid promoter.

In one embodiment, the promoter is a thyroxine binding globulin (TGB) promoter. In a further embodiment, the TGB promoter comprises the nucleic acid sequence set forth in SEQ ID NO: 18. In another embodiment, the TGB promoter consists of the nucleic acid sequence set forth in SEQ ID NO:18. In yet another embodiment, the promoter is a modified thyroxine binding globulin (TBG) promoter. For example, the modified TBG promoter, in one embodiment, is a shorted version, termed TBG-S 1. In a further embodiment, the TGB-S 1 promoter comprises the nucleic acid sequence of SEQ ID NO:19.

In another embodiment, the promoter is a transthyretin (TTR) promoter. In a further embodiment, the TTR promoter comprises the nucleic acid sequence set forth in SEQ ID NO: 20. In another embodiment, the TTR promoter consists of the nucleic acid sequence set forth in SEQ ID NO:20.

In another embodiment, the promoter is a chicken β-actin promoter. In a further embodiment, the chicken β-actin promoter comprises the nucleic acid sequence set forth in SEQ ID NO:21. In another embodiment, the chicken β-actin promoter consists of the nucleic acid sequence set forth in SEQ ID NO:21.

In some embodiments, an AAV vector genome of the present disclosure comprise a muscle-specific promoter that is operably linked to the transgene to drive high-level and tissue-specific expression in muscle cells. For example, muscle specific promoters of the present disclosure include, but are not limited to, promoters selected from: desmin (DES, also known as CSM1 or CSM2) promoter, the alpha 2 actinin (ACTN2, also known as CMD1AA) promoter, the filamin-C (FLNC, also known as actin-binding-like protein (ABLP), filamin-2 (FLN2), ABP-280, ABP280A, ABPA, ABPL, MFM5 or MPD4) promoter, the sarcoplasmic/endoplasmic reticulum calcium ATPase 1 (ATP2A1, also known as ATP2A or SERCA1) promoter, the troponin I type 1 (TNNI1, also known as SSTNI or 25TTNI) promoter, the myosin-1 (MYH1) promoter, the phosphorylatable, fast skeletal muscle myosin light chain (MYLPF) promoter, myosin 1 (MYH1, also known as MYHSA1, MYHa, MyC-2X/D or MyHC-2x) promoter, the alpha-3 chain tropomyosin (TPM3, also known as CFTD, NEM1, OK/Scl.5, TM-5, TM3, TM30, TM30 nm, TM5, TPMsk3, TRK, h TM5 or hscp30) promoter, the ankyrin repeat domain-containing protein 2 (ANKRD2, also known as ARPP) promoter, the myosin heavy-chain (MHC) promoter, the myosin light-chain (MLC) promoter, the muscle creatine kinase (MCK) promoter, synthetic muscle promoters as described in Li et al. (1999. Nat Biotechnol. 17:241-245), such as the SPc5-12 promoter, the muscle creatine kinase (MCK) promoter, the dMCK promoter, the tMCK promoter consisting of respectively, a double or triple tandem of the MCK enhancer to the MCK basal promoter as described in Wang et al. (2008. Gene Ther. 15:1489-1499) and hybrid promoters such as the hybrid alpha-myosin heavy chain enhancer/MCK enhancer (MHCK7; 770 bp); the MCK-C5-12 promoter as described in Wang et al. (2008. Gene Ther. 15:1489-1499) and the cardiac and skeletal muscle-specific myosin chaperone Unc45b (195 bp) promoter as described in Rudeck S et al. (2016, Genesis. 54(8): 431-8). Non-limiting examples of heart-specific promoters include the calsequestrin 2 (also known as PDIB2, FLJ26321, FLJ93514 or CASQ2 (GeneID 845 for the human gene)) promoter, the ankyrin repeat domain 1 (also known as cardiac ankyrin repeat protein) promoter, the cytokine inducible nuclear protein promoter; the liver ankyrin repeat domain 1 (ANKRD1; GeneID 27063 for the human gene) promoter; the myosin, light chain 2, regulatory, cardiac, slow (MYL2; GeneID 4633 for the human gene) promoter; the myosin, light chain 3, alkali; ventricular, skeletal 10 slow (MYL3; GeneID 4634 for the human gene) promoter; the bromodomain containing 7 (also known as BP75, CELTIX1, NAG4 (BRD7; GeneID 29117 for the human gene)) promoter; the alpha myosin heavy chain (aMHC) promoter; the cardiac troponin C promoter and the promoter of the cardiac sodium-calcium exchanger (NCX1), which confers cardiac specificity.

Another suitable promoter for use herein includes, but is not limited to, the CAG promoter, which comprises (C) the cytomegalovirus (CMV) early enhancer element, (A) the promoter, the first exon and the first intron of chicken beta-actin gene, and (G) the splice acceptor of the rabbit β-globin gene. See, e.g., Alexopoulou et al. (2008). BMC Cell Biology 9.1:2, incorporated by reference herein in its entirety for all purposes.

In one embodiment, the promoter is an AIAT promoter combined with an ApoE enhancer, sometimes referred to as ApoE/A1AT. In another embodiment, the promoter for use in a vector genome of the disclosure is the liver specific promoter LSP (TH-binding globulin promoter/alphal-microglobulin/bikunin enhancer). Other suitable promoters include human albumin, and hepatitis B virus core promoter.

In some embodiments, an AAV vector genome described herein comprises a MHCK7 promoter.

In some embodiments, AAV vector genomes of the present disclosure comprise an intron. The intron, in one embodiment, is a SV40 intron. The SV40 intron is a commonly used regulatory element in gene therapy vectors and enhances translation and stability of the expressed RNA transcript.

In certain embodiments, the intron, e.g., SV40 intron, is downstream (i.e., 3′) of the promoter and upstream (i.e., 5′) of the transgene. In other embodiments, the SV40 intron can be downstream (i.e., 3′) of the transgene.

In one embodiment, the vector genome for use herein comprises an intron, and the intron is selected from CBA, human β-globin, IVS2, SV40, bGH, α-globulin, β-globulin, collagen, ovalbumin, or p53.

In some embodiments, AAV vector genomes of the present disclosure comprise a nucleic acid sequence encoding a poly(A) tail.

In one embodiment, a suitable polyA tail is derived from bovine growth hormone (bGH), human growth hormone (hGH), SV40, rabbit beta globin, modified RGB (mRGB) or thymidine kinase (TK). The poly(A) tail in one embodiment is a SV40-poly(A) tail. The poly(A) tail, in another embodiment, is a bovine growth hormone (bGH)-poly(A) tail. In even another embodiment, the polyA is a synthetic poly(A) or from bovine growth hormone (bGH), human growth hormone (hGH), SV40, rabbit β-globin (RGB), or modified RGB (mRGB).

In some embodiments, AAV vector genomes of the present disclosure comprise a nucleic acid sequence encoding an SV40 poly(A) tail signal having at least 80%, 85%, 90%, 91%, 92%, 93%, 94%, 95%, 96%, 97%, 98%, 99%, or 100% sequence identity to SEQ ID NO: 22. In some embodiments, the nucleic acid sequence encoding the SV40 poly(A) tail signal comprises the sequence set forth in SEQ ID NO:22. In some embodiments, the nucleic acid sequence encoding the SV40 poly(A) tail signal consists of the sequence set forth in SEQ ID NO: 22.

In some embodiments, an AAV vector genome of the present disclosure comprises one or more enhancer sequence(s). An enhancer sequence, in one embodiment, can increase the level of transcription of the transgene, for example, by serving as a binding site for transcription factors and co-regulators that assist in DNA looping and recruitment of the transcriptional machinery to promoters.

In some embodiments, the enhancer is downstream (i.e., 3′) of the 5′ ITR and upstream (i.e., 5′) of the promoter. In some embodiments, the enhancer is downstream (i.e., 3′) of the promoter and upstream (i.e., 5′) of the transgene. In some embodiments, the enhancer is downstream (i.e., 3′) of the transgene and upstream (i.e., 5′) of the 3′ UTR.

In some embodiments, recombinant AAV vector genomes of the present disclosure comprise an enhancer that significantly promotes the transcription of a transgene in muscle cells, e.g., skeletal and/or cardiac muscle cells.

In some embodiments, recombinant AAV vectors described herein comprise a skeletal-cis-regulatory module 4 (SK-CRM4) enhancer.

In yet another embodiment, an AAV vector genome of the present disclosure comprises a cytomegalovirus (CMV) enhancer nucleic acid sequence. In a further embodiment, the CMV enhancer is upstream of a promoter sequence. In a further embodiment, the CMV enhancer comprises the nucleic acid sequence of SEQ ID NO:23. In another embodiment, the CMV enhancer consists of the nucleic acid sequence of SEQ ID NO:23. In a further embodiment, the CMV enhancer is upstream of a promoter sequence.

In one embodiment, the enhancer is a cis regulatory module 8 (CRM8) enhancer. In a further embodiment, the CRM8 enhancer comprises the nucleic acid sequence set forth in SEQ ID NO:24. In another embodiment, the CRM8 enhancer consists of the nucleic acid sequence set forth in SEQ ID NO:24. The CRM8 enhancer, in one embodiment, is upstream of a promoter element.

In another aspect of the disclosure, a DNA vector (e.g., DNA plasmid) is provided, comprising an expression cassette described herein. DNA vectors such as DNA plasmids containing one or more of the expression cassettes provided herein, including an expression cassette comprising an AAV genome, can be constructed according to methods known to those of skill in the art. For example, the gateway cloning method (Invitrogen), restriction cloning, or Gibson assembly can be employed.

In one embodiment, the gateway cloning method (Invitrogen), is used to construct plasmids with one or more of the expression cassettes provided herein. Entry clones can be constructed by flanking an expression cassette provided herein, or a portion thereof, with attB sites. BP clonase can then be used to insert the entry clone into a donor vector between attP sites.

In another embodiment, restriction cloning is used to insert an expression cassette, or a portion thereof, into a DNA plasmid.

In some instances, a DNA plasmid may already include one or more components of an expression cassette. For example, one DNA plasmid amenable for use herein, e.g., to subclone expression cassettes into, or to subclone a portion of an expression cassette into, is the expression vector pNoc ox Venus. See, e.g., Zienkiewicz et al. (2017). Biotechnol. Biofuels 10:8, incorporated by reference herein in its entirety. This vector possesses one expression cassette consisting of the promoter of the lipid droplet surface protein (LDSP), the HygR gene for resistance to hygromycin B and the nopaline synthase terminator and a second cassette for the expression of the reporter gene controlled by the elongation factor (EF) promoter and terminated by the LDSP terminator. In one embodiment, the reporter gene is excised from the pNoc ox Venus vector and a gene encoding a viral assembly protein is inserted in its place.

In some embodiments, an expression cassette is cloned into a DNA vector, linearized and introduced into the Nannochloropsis. In one embodiment, the expression cassette is subsequently integrated into the Nannochloropsis genome. In one embodiment, the expression cassette is integrated into the Nannochloropsis genome at a specific site, e.g., by homologous recombination. In another embodiment, the expression cassette is randomly integrated into the Nannochloropsis genome.

In some embodiments, to increase growth rate and chlorophyll production, the Nannochloropsis microalgae includes one or more expressible genetic constructs capable of driving the expression of one or more polynucleotides encoding glutamine phenylpyruvate transaminase (GPT), and in some embodiments, one or more polynucleotides encoding glutamine synthetase and GPT.

In another aspect of the disclosure, a Nannochloropsis packaging cell is provided, which is sometimes referred to herein as a host cell. The Nannochloropsis packaging cell in the embodiments described herein, comprises an expression cassette of the disclosure.

The Nannochloropsis host cell in one embodiment, comprises an expression cassette comprising (i) a first Nannochloropsis-endogenous promoter comprising a first end and a second end, (ii) an AAV cap coding sequence, (iii) a first Nannochloropsis-endogenous 3′ untranslated region (UTR) downstream of the AAV cap coding sequence and operably linked thereto, (iv) an antibiotic resistance gene coding sequence, wherein the antibiotic resistance gene coding sequence is operably linked to the first Nannochloropsis-endogenous 3′ untranslated region (UTR) or a second Nannochloropsis-endogenous 3′ UTR. The antibiotic resistance gene coding sequence is operably linked to the first Nannochloropsis-endogenous promoter or alternatively, is operably linked to a second Nannochloropsis-endogenous promoter comprising a first end and a second end. In one embodiment, the expression cassette is present in a DNA plasmid prior to integration into a Nannochloropsis host genome. The packaging cell, in a further embodiment, comprises, an expression cassette comprising (i) a first Nannochloropsis-endogenous promoter comprising a first end and a second end, (ii) an AAV rep coding sequence, (iii) a first Nannochloropsis-endogenous 3′ untranslated region (UTR) downstream of the AAV rep coding sequence and operably linked thereto, (iv) an antibiotic resistance gene coding sequence, wherein the antibiotic resistance gene coding sequence is operably linked to the first Nannochloropsis-endogenous 3′ untranslated region (UTR) or a second Nannochloropsis-endogenous 3′ UTR. The antibiotic resistance gene coding sequence is operably linked to the first Nannochloropsis-endogenous promoter or alternatively, is operably linked to a second Nannochloropsis-endogenous promoter comprising a first end and a second end. In one embodiment, the expression cassette is present in a DNA plasmid prior to integration into a Nannochloropsis host genome.

In some embodiments, the AAV cap coding sequence comprises the nucleic acid sequence of SEQ ID NO: 31 (AAV9 VP1), 32 (AAV9 VP2), 33 (AAV9 VP2) or 34 (AAV9 VP3). The rep gene coding sequence, in one embodiment, comprises the nucleic acid sequence of SEQ ID NO:60 (Rep52) or SEQ ID NO:61 (Rep78). The AAP gene coding sequence, in one embodiment, comprises the nucleic acid sequence of SEQ ID NO:57, 58, 59 or 76. DBP and E4orf6 coding sequences, in one embodiment, comprises the nucleic acid sequence of SEQ ID NOS: 62 and 63, respectively. The antibiotic gene coding sequence in one embodiment, comprises the nucleic acid sequence selected of SEQ ID NO: 51, 52, 53, 54, 55 or 56.

In one embodiment, the Nannochloropsis packaging cell produces a recombinant virus, e.g., a recombinant AAV. In some embodiments, the recombinant virus is a replication-restricted poxvirus (e.g., MVA, NYVAC, FP9, TROVAC and ALVAC strains), replication-restricted adenovirus (e.g., replication-restricted Ad5 and ONYX-015), influenza virus, replication-incompetent alphavirus (e.g., Venezuelan equine encephalitis virus (VEE), Sindbis virus (SIN), Semliki forest virus (SFV) and VEE-SIN chimeras, parvovirus (e.g., AAV), lentivirus, attenuated flavivirus, or herpesvirus. In some embodiments, the recombinant virus is a recombinant gene therapy vector including, but not limited to, a parvovirus (e.g., AAV), adenovirus (e.g., Ad5 and ONYX-015), retrovirus or herpes simplex virus. In one embodiment, the recombinant virus is a recombinant vaccine vector.

Examples of Nannochloropsis microalgae for use as a packaging cell, and for producing recombinant virus, include Nannochloropsis australis, Nannochloropsis gaditana, Nannochloropsis granulata, Nannochloropsis limnetica, Nannochloropsis oceanica, Nannochloropsis oculate, or Nannochloropsis salina. In a preferred embodiment, the Nannochloropsis microalga is Nannochloropsis oceanica.

In one embodiment, the Nannochloropsis microalga packaging cell is one of the axenic Nannochloropsis strains obtained from the National Center for Marine Algae and Microbiota (NCMA, formerly CCMP, Maine, USA).

In one embodiment, the Nannochloropsis microalga packaging cell is N. cf. strain CCMP821, N. cf. strain CCMP2001, N. cf. strain CCMP1997, N. cf. strain CCMP2904, N. cf. strain CCMP1780, Nannochloropsis sp. strain CCMP531, N. granulata strain CCMP535, N. granulata strain CCMP534, N. granulata strain CCMP529, N. limnetica strain CCMP2392, N. limnetica strain CCMP2271, N. limnetica strain CCMP2267, N. limnetica strain CCMP2253, N. oculata strain CCMP525, N. limnetica strain CCMP505, N. limnetica strain CCMP2272, N. limnetica strain CCMP2260, N. oculata strain CCMP2195, N. oceanica strain CCMP1779, or N. granulata strain CCMP1662.

In one embodiment, the Nannochloropsis microalga packaging cell is N. granulata strain CCMP535, N. granulata strain CCMP534, N. granulata strain CCMP529 or N. granulata strain CCMP1662.

In one embodiment, the Nannochloropsis microalga packaging cell is N. limnetica strain CCMP2392, N. limnetica strain CCMP2271, N. limnetica strain CCMP2267, N. limnetica strain CCMP2253, N. oculata strain CCMP525, N. limnetica strain CCMP505, N. limnetica strain CCMP2272 or N. limnetica strain.

In a preferred embodiment, the Nannochloropsis microalga packaging cell is N. oceanica. In a further preferred embodiment, the N. oceanica is N. oceanica strain CCMP1779.

In one embodiment, the Nannochloropsis packaging cell produces a recombinant AAV (rAAV). Production of rAAV requires that the following components are present within a single cell: a rAAV vector genome, AAV rep and cap genes separate from (i.e., not in) the rAAV vector genome, and helper virus functions (e.g., adenovirus, E1-deleted adenovirus or herpesvirus helper virus functions). The rep and cap genes as well as helper virus functions in one embodiment, are present in one or more of the expression cassettes described herein. The AAV rep and cap genes may be from any AAV serotype for which recombinant virus can be derived and may be from a different AAV serotype than the rAAV vector genome ITRs, including, but not limited to, AAV serotypes AAVrh.8, AAVrh.10, AAVrh.74, AAV1, AAV2, AAV3, AAV4, AAV5, AAV6, AAV7, AAV8, AAV9, AAV10, AAV11, AAV12, AAV13 or a variant of one of the foregoing. In a preferred embodiment, the rAAV vector genome ITRs are AAV2 ITRs.

The recombinant virus produced by the Nannochloropsis provided herein include, but is not limited to, adeno-associated virus (AAV). In a further embodiment, the AAV is AAVrh.8, AAVrh.10, AAVrh.74, AAV1, AAV2, AAV3, AAV4, AAV5, AAV6, AAV7, AAV8, AAV9, AAV10, AAV11, AAV12, AAV13 or a variant thereof. In yet even a further embodiment, the recombinant virus produced by the Nannochloropsis is recombinant AAV9. As such, the Nannochloropsis in embodiments described herein is an AAV (e.g., AAV9) packaging cell. A Nannochloropsis packaging cell contains components for recombinant virus particle production, e.g., rAAV production.

In embodiments described herein, a Nannochloropsis microalga packaging cell is engineered to comprise the components needed for assembly of the recombinant virus, e.g., a recombinant AAV (e.g., AAV9) encapsidating a vector genome. For example, expression cassettes comprising rep and cap coding sequences can be introduced into the Nannochloropsis cell together with an expression cassette comprising an AAV genome for packaging within the virus, e.g., AAV. As will be understood by those skilled in the art, in this embodiment, the Nannochloropsis thus comprises (i) a recombinant vector genome (e.g., a vector genome comprising a transgene encoding an immunogen, a therapeutic protein, an inhibitory RNA or a therapeutic RNA), (ii) DNA encoding rep and cap proteins, and in some embodiments, DNA encoding helper virus components/functions. Expression cassette architectures amenable for use herein are described throughout the specification. See, e.g., FIGS. 1-5. The Nannochloropsis comprising the aforementioned components can be grown, and recombinant virus produced.

Expression cassettes provided herein can be transiently or stably transformed into a Nannochloropsis host. Typically, stable transformation involves the integration of transformed DNA into the host cell genome, allowing transformed cells to pass this DNA to their progeny. Expression cassettes that are stably transformed into Nannochloropsis can be integrated randomly into the host genome by nonhomologous end joining (NHEJ) or at a specific site via homologous recombination. In one embodiment, an expression cassette is introduced into the Nannochloropsis genome by CRISPR/homology directed repair.

In one embodiment, an expression cassette comprising AAV cap gene coding sequences (VP1, VP2, VP3) and an expression vector comprising AAV rep gene coding sequences (e.g., Rep78, Rep52) are stably transformed into a Nannochloropsis host, e.g., a Nannochloropsis oceanica host, the host can be considered a general production platform for AAV particles. Once the production platform is established, DNA plasmids/expression cassettes comprising AAV vector genomes, e.g., ITR flanked therapeutic transgenes can be transformed into the production platform. In this regard, the production platform can be used to package different AAV genomes.

Alternatively, an AAV vector genome may be present in one of the rep or cap expression cassettes, and transformed into the host cell.

In some embodiments, a plasmid comprising one or more of the expression cassettes provided herein is linearized and introduced into the Nannochloropsis via transformation. See, e.g., FIGS. 1-6 for various architectures of expression cassettes that can be used herein.

Expression cassettes can be introduced into Nannochloropsis species either transiently (e.g., episomal plasmid transformation) or stably (i.e., via random genomic integration or site-specific genomic integration).

Transient introduction or transient transformation, as used herein, refers to embodiments where one or more expression cassettes are introduced into the Nannochloropsis (e.g., via a DNA plasmid), but the foreign genes of the expression cassette(s) are not integrated into the Nannochloropsis genome. In one embodiment, a transiently introduced expression cassette is stable for a plurality of generations while under antibiotic selection for the antibiotic resistance cassette that the expression cassette, or DNA plasmid containing the expression cassette, carries. In transiently introduced plasmids, foreign genes will be gradually and totally lost with the growth and division of host cells. The loss of foreign genes, in one embodiment, occurs after at least about a dozen generations, after at least about two dozen generations, after at least about three dozen generations, after at least about four dozen generations, after at least about five dozen generations, after at least about six dozen generations, after at least about seven dozen generations, after at least about eight dozen generations, after at least about nine dozen generations, after at least about ten dozen generations, or after at least about eleven dozen generations.

In one embodiment, an expression cassette provided herein is transiently introduced into a Nannochloropsis packaging cell.

In a preferred embodiment, the Nannochloropsis packaging cell is stably transformed with the expression cassette provided herein. In a further embodiment, an expression cassette is integrated randomly into the genome of a Nannochloropsis packaging cell.

In another embodiment, an expression cassette is integrated in a site-specific manner into the genome of a Nannochloropsis packaging cell, e.g., via the use of homology arms flanking the expression cassette.

In one embodiment, the expression cassette is genomically integrated into the Nannochloropsis host in a site specific manner via the use of a first and second homology arm on each end of the expression cassette. A homology arm, in one embodiment, is from about 100 to about 1000 nucleotides in length, for example, from about 100 to about 900 nucleotides in length, from about 100 to about 800 nucleotides in length, from about 100 to about 700 nucleotides in length, from about from about 100 to about 600 nucleotides in length, or from about 100 to about 500 nucleotides in length. A homology arm, in another embodiment, is from about 200 to about 1000 nucleotides in length, for example, from about 300 to about 1000 nucleotides in length, from about 400 to about 1000 nucleotides in length, from about 500 to about 1000 nucleotides in length, from about 600 to about 1000 nucleotides in length, or from about 700 to about 1000 nucleotides in length. In one embodiment, the first and second homology arms are each about 1000 nucleotides in length. In a preferred embodiment, the first and second homology arms are about the same length. The homology arms, in one embodiment, comprise the nucleic acid sequences of SEQ ID NO:66 and SEQ ID NO:67.

Genome integration, in one embodiment, is random genome integration. In another embodiment, genome integration is site-specific genome integration. Site-specific genome integration, e.g., via homologous recombination, or CRISPR/homology directed repair, in one embodiment, is nuclear genomic integration. In another embodiment, genomic integration is mitochondrial genomic integration or chloroplast genomic integration. In one embodiment, a DNA plasmid encoding a viral vector genome is transiently introduced into the Nannochloropsis packaging cell. In another embodiment, a DNA plasmid encoding a viral vector genome is stably introduced into the Nannochloropsis packaging cell and integrated into the host genome. Transformation techniques and protocols are well known to those of ordinary skill in the art and are amenable for use herein.

Expression cassettes comprising AAV genomes, polynucleotides encoding antibiotic resistance, cap and rep coding sequences, helper functions, and DNA vectors (e.g., DNA plasmids) comprising the same, are stably or transiently introduced into the Nannochloropsis species by standard transformation methods known to those skilled in the art, such as electroporation, vortexing cells in the presence of exogenous DNA, acid washed beads, polyethylene glycol, plasmid conjugation and/or microparticle bombardment. See, for example, Hallmann, Transgenic Plant Journal, 1:81-98 (2007). The transformed Nannochloropsis are recovered on a solid nutrient media or in liquid media. See Vieler et al. (2012). PLOS Genetics 8(11):e1003064. See also, Harris, Annual Review of Plant Physiology and Plant Molecular Biology 52:363-406 (2001). The disclosure of each of the references cited in this paragraph is incorporated by reference herein in its entirety for all purposes.

In one embodiment, transformation is carried out according to a method set forth in U.S. Pat. No. 8,119,859 or U.S. Patent Application Publication No. 2009/0317847, the disclosure of each of which is incorporated by reference herein in their entireties for all purposes.

In one embodiment, transformation is carried out via (i) agitation with glass beads, (ii) electroporation, or (iii) particle bombardment.

In a preferred embodiment, DNA is introduced into a Nannochloropsis host via electroporation. In one embodiment, transformation comprises electroporating the Nannochloropsis with linearized vector DNA. In one embodiment, vectors/plasmids are linearized by restriction digestion and optionally further purified, e.g., with ethanol precipitation followed by resuspension in water. In one embodiment, electroporating further comprises electroporating with a carrier DNA such as heat denatured salmon sperm DNA.

In one embodiment, the Nannochloropsis is grown to mid exponential growth phase prior to transformation. Cell concentration can then optionally be adjusted prior to performing electroporation. In a further embodiment, the Nannochloropsis is Nannochloropsis oceanica.

In one embodiment, the one or more DNA plasmids are introduced into the Nannochloropsis via electroporation. In a further embodiment, electroporation is carried out at a field strength of from about 10,000 Vcm⁻¹ to about 13,000 Vcm⁻¹. Electroporation is carried out in one embodiment, by first linearizing nucleic acid constructs (e.g., expression cassettes, DNA plasmid vectors). In a further embodiment, linearization is carried out with a restriction enzyme. In one embodiment, electroporation is carried out using a BIORAD Gene Pulser Xcell system, or a comparable system.

After electroporation, cells in one embodiment, cells are collected in f/2 media in a vessel. Cells can be optionally centrifuged and resuspended and plated on f/2 plated containing the respective antibiotics, and colonies picked 3-8 weeks after transformation. Optionally, cells can be incubated in low light overnight and then plated.

For example, in one embodiment, agitation with glass beads or electroporation is used to transform Nannochloropsis microalgae that lack or have weakened cell walls. In another embodiment, particle bombardment is employed to transform a Nannochloropsis species with a hard cell wall.

In another embodiment, the one or more DNA plasmids are introduced into the Nannochloropsis via agitation with glass beads.

In another embodiment, the one or more DNA plasmids are introduced into the Nannochloropsis via particle bombardment. In a further embodiment, DNA plasmids are precipitated onto gold particles, and particle bombardment is employed to transform chloroplasts of the Nannochloropsis strain. See, e.g., Rasala et al. (2010). Plant Biotechnol J 8 (6): 719-733, the content of which is incorporated by reference herein in its entirety for all purposes.

In some embodiments, a gene silencing inhibitor is also introduced into the Nannochloropsis microalgae. A gene silencing inhibitor is a peptide that induces relaxation of nucleosomes in the algae's nucleus. Gene silencing inhibitors include histone acetyl transferases (HATs) and other peptides that modify elements of the nucleosome, causing the chromatin structure to relax and to allow transcription factors to access the gene of interest. HAT proteins and the HAT domains of p300 and of other HAT proteins are known to cause histone acetylation and can be utilized in the disclosure. In some embodiments, a p300 protein is used as a gene silencing inhibitor.

In some embodiments, the gene silencing inhibitor is functionally tethered or, fused to a DNA binding protein or domain thereof. The DNA binding protein or domain binds to a particular DNA sequence, bringing the gene silencing inhibitor to its histone target at a location in the vicinity of the binding site and thereby inducing relaxation of the nucleosome at that genetic location. As the nucleosome relaxes, the nearby DNA sequence is exposed to transcription factors and is more actively transcribed. Examples of proteins targeting specific DNA motifs applicable to this disclosure include the Gal4 protein and Early Growth Response Protein 1. DNA binding site motifs for these proteins are known. Likewise, the binding domains of these as well as the LexA protein are known and are used, instead of the full-length protein. See, e.g., Young (1998). Biol. Reprod., 58, pp. 302-311 and Joung et al. (2000). Proc. Natl. Acad. Sci. 97, pp. 7382-7387. LexA is a gene of bacterial origin. LexA proteins or genes are not known in algae. Thus, it is unlikely that the Nannochloropsis genome will contain the DNA binding sequence of LexA. The function of LexA in the context of the disclosure is to bind a particular DNA sequence LexA binding sites are found upstream promoters in a number of microorganisms. The binding domain of the LexA protein is known and, for the purpose of the disclosure, it is preferred to employ only the binding domain (Protein ID: 2293118 from NCBI Database).

In some embodiments, the activity of proteases, such as ATP-dependent proteases, is limited in the Nannochloropsis packaging cell.

In another aspect, a Nannochloropsis microalga producing a recombinant virus is provided herein. In a related aspect, a method of producing a recombinant virus in a Nannochloropsis host is provided. The method comprises in part, growing a Nannochloropsis microalgae that has been transformed with one or more of the expression cassettes provided herein, e.g., one or more expression cassettes encoding the viral assembly proteins and viral vector genome, and producing the recombinant virus in the Nannochloropsis. In some embodiments the methods further comprise purifying or recovering the recombinant virus. In some embodiments, the recombinant virus is a viral vaccine vector. In some embodiments, the recombinant virus is a viral gene therapy vector.

In some embodiments, the Nannochloropsis microalga of the present disclosure produces a larger quantity of recombinant virus in the same amount of time, as compared to a counterpart microalga. In a further embodiment, the counterpart microalga is Chlamydomonas reinhardtii. In even a further embodiment, the recombinant virus is recombinant AAV. In yet even a further embodiment, the recombinant AAV is recombinant AAV9. In one embodiment, the Nannochloropsis microalgae produces at least about 10% more, at least about 20% more, at least about 30% more, at least about 40% more or at least about 50% more recombinant virus in the same amount of time, as compared to a counterpart microalga. In a further embodiment, the counterpart microalga is Chlamydomonas reinhardtii. In even a further embodiment, the recombinant virus is recombinant AAV. In yet even a further embodiment, the recombinant AAV is recombinant AAV9.

In one embodiment, a method is provided for making a Nannochloropsis microalgae that produces a recombinant adeno-associated virus (rAAV). The method comprises transforming a Nannochloropsis microalgae with the expression cassette of the disclosure comprising one or more cap gene coding sequences, e.g., an expression cassette comprising a VP1, VP2 and VP3 coding sequences, and an AAP coding sequence, to produce transformed Nannochloropsis microalgae, selecting for the transformed Nannochloropsis microalgae and growing the transformed Nannochloropsis microalgae. Next, the transformed Nannochloropsis microalgae is transformed with an expression cassette comprising one or more rep gene coding sequences to produce a second transformed Nannochloropsis microalgae. The second transformed Nannochloropsis microalgae is selected for, and the selected microalgae is then grown to produce a Nannochloropsis microalgae that produces a recombinant adeno-associated virus (rAAV).

In one embodiment, the Nannochloropsis is grown in f/2 medium, e.g., in a vessel with or without agitation. Guillard and Ryther (1962). Canadian J. of Microbiology 8, pp. 229-239, incorporated by reference herein in its entirety for all purposes. In one embodiment, Nannochloropsis are grown in batch culture. In a further embodiment, the batch culture is in a photobioreactor. In one embodiment, Nannochloropsis cultivation is carried out in a continuous manner. In another embodiment, Nannochloropsis cultivation is carried out in a non-continuous manner.

The f/2 media, in one embodiment, is prepared with artificial sea water (ASW), e.g., at from about 2% (w/v) to about 10% (w/v), from about 2% (w/v) to about 5% (w/v), from about 2% (w/v) to about 4.5% (w/v), or from about 2% (w/v) to about 4% (w/v), from about 2% (w/v) to about 3.5% (w/v), or from about 2% to about 3% (w/v). In one embodiment, the f/2 medium is supplemented, for example, with trace metals, vitamins, phosphate solution and/or a nitrogen source. The nitrogen source, in one embodiment, is NH₄Cl, KNH₄ or NaNO₃. In another embodiment, the nitrogen source is urea. In yet another embodiment, the nitrogen source is peptone.

In one embodiment, the Nannochloropsis is grown in a vessel containing f/2 media under light: dark cycles. In one embodiment, the light: dark cycle is 16 hr. light: 8 hr. dark, 14 hr. light: 10 hr. dark, 12 hr. light: 12 hr. dark, or 8 hr. light: 12 hr. dark. The light intensity, in one embodiment is kept at from about 70 μmol s⁻¹m⁻² to about 130 μmol s⁻¹m⁻². The light intensity, in one embodiment is kept at from about 30 μmol s⁻¹m⁻² to about 80 μmol s⁻¹m⁻². The light intensity, in one embodiment is about 40 μmol s⁻¹m⁻². The light intensity, in one embodiment is about 30 μmol s⁻¹m⁻². The light intensity, in one embodiment is about 80 μmol s⁻¹m⁻².

In one embodiment, the Nannochloropsis is grown in a vessel containing f/2 media under continuous light. The light intensity, in one embodiment is kept at from about 30 μmol s⁻¹m⁻² to about 130 μmol s⁻¹m⁻². The light intensity, in one embodiment is kept at from about 30 μmol s⁻¹m⁻² to about 80 μmol s⁻¹m⁻². The light intensity, in one embodiment is about 40 μmol s⁻¹m⁻². The light intensity, in one embodiment is about 30 μmol s⁻¹m⁻². The light intensity, in one embodiment is about 60 μmol s⁻¹m⁻². The light intensity, in one embodiment is about 80 μmol s⁻¹m⁻².

In one embodiment, the Nannochloropsis culture is stirred during the light period. In one embodiment, the Nannochloropsis culture is supplied with a CO₂/air mixture during the light period. In one embodiment, to compensate for medium acidification by CO₂ bubbling, the pH of the growth medium is adjusted to a pH of about 9 and subsequently dropped to pH of about 8 after 2 hr. of bubbling.

In one embodiment, the Nannochloropsis is cultured at about 25° C.±1, at about 25° C.±2, at about 24° C.±1, at about 24° C.±2, at about 23° C.±1, at about 23° C.±2, at about 22° C.±2, or at about 22° C.±1.

General principles of rAAV production are reviewed in, for example, Carter, Current Opinions in Biotechnology, 1533-1539 (1992); and Muzyczka, Curr. Topics in Microbial. and Immunol., 158:97-129 (1992). Various approaches are described in Ratschin et al., Mol. Cell. Biol., 4:2072 (1984); Hermonat et al., Proc. Natl. Acad. Sci. USA, 81:6466 (1984); Tratschin et al., Mol. Cell. Biol., 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 1995/13392; WO 1996/17947; WO 1999/011764; WO 1997/09441; WO 1997/08298; WO 1997/21825; WO 1997/06243; WO 1999/11764; Perrin et al., Vaccine, 13:1244-1250 (1995); Paul et al., Human Gene Therapy, 4:609-615 (1993); Clark et al., Gene Therapy, 3:1124-1132; U.S. Pat. Nos. 5,786,211; 5,871,982; and 6,258,595, the disclosure of each of which are incorporated by reference herein in their entireties for all purposes. It is contemplated that the AAV production components used in these approaches can be adapted for recombinant virus production in Nannochloropsis microalgal cells. Similarly, it is contemplated that production components known in the art for production of other recombinant viruses can be used or adapted as described herein to produce the recombinant viruses in Nannochloropsis microalgae.

The recombinant virus produced by the Nannochloropsis microalgae may be purified by methods standard in the art. For example, rAAV may be purified by methods standard in the art such as by column chromatography, tangential flow filtration and/or cesium chloride gradients.

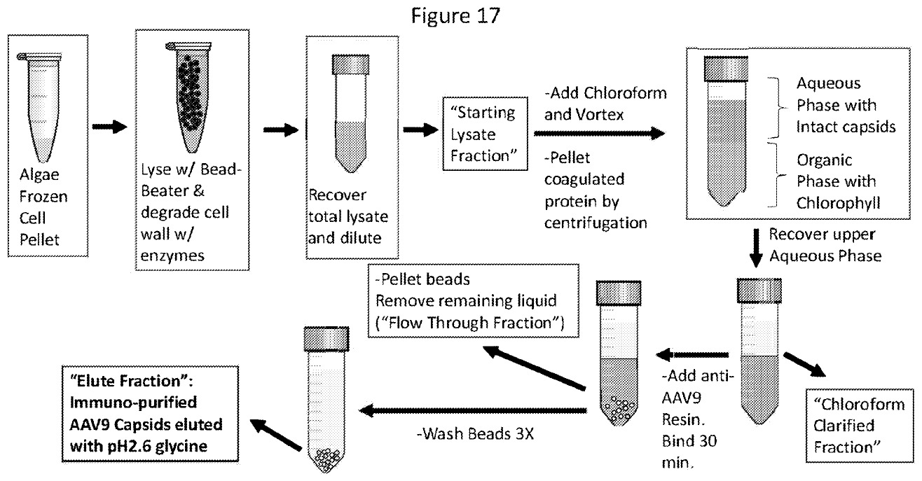

In some embodiments, AAV particles are harvested from Nannochloropsis via the method outlined in FIG. 17. For each sample, wet-biomass cell pellet is resuspended in room temperature (RT) Algae Virus-like particle Isolation Buffer (AVIB) (500 mM NaCl, 100 mM Trisodium citrate pH 8.2, 1 mM EDTA, 0.3% vol./vol. NP-40 and 0.3% wt./vol sodium Deoxycholate detergent supplemented with 1× Halt protease and phosphatase inhibitor (Thermofisher) and 1 mM DTT). AAV Dilutant Buffer (150 mM NaCl, 20 mM sodium phosphate buffer pH 7.2, 0.01% Pluronic F-68) is added to each sample. The suspended sample is loaded into Lysing Matrix E (MP Biomedicals) tubes and disrupted in a Qiagen TissuelyserII. Bead-beating cycles with 1 min.-3 min. rest times between each cycle is used to lyse the cells. The resulting emulsion is resolved by centrifuging at 3000 rcf followed by addition of cell wall enzyme master mix containing 0.4 mg/mL Cellulase R-10, 0.4 mg/mL Macerozyme R-10 (both from Goldbio), 5000 U/mL of Basemuncher nuclease (Abcam), and 200 mM MgCl₂ in 100 mM citrate buffer pH 3.8. The algae lysate, in one embodiment, is then put on an end-over-end rotator to degrade the cell walls. The lysate in a further embodiment, is snap-frozen in crushed dry ice, quick thawed and bead-beat again to provide a frothy-emulsion.

The frothy-emulsion in one embodiment, is resolved by centrifuging at 3000 rcf. Supernatant in a further embodiment, is recovered into a new tube. The remaining pellet in a further embodiment, is re-extracted by adding AVIB and inverting the tube about 10× before re-pelleting the cell debris by centrifuging. Additional re-extracted lysate is then pooled with the first extraction and molecular biology grade water is added to the extraction. Cell wall debris is then removed by centrifugation, providing the Starting Lysate Fraction shown in FIG. 17.

The clarified supernatant (Starting Lysate Fraction in FIG. 17) in one embodiment, is mixed with ethanol stabilized chloroform and vortexed. The emulsion in a further embodiment, is resolved by centrifugation. The de-pigmented and clarified aqueous layer, containing intact capsids, in a further embodiment, is recovered into a new tube. The clarified aqueous layer is then applied to 50 μL packed volume of affinity capture beads for the appropriate AAV serotype. For example, in the case of AAV9, POROS™ CaptureSelect™ AAV9 Affinity Resin beads can be employed.

The single-chain camelid VH (heavy chain variable region) antibodies conjugated to the POROS anti-AAV9 resin selectively and specifically recognize the five-fold symmetry “peak” of AAV9 (Mietzsch et al. (2020). Molecular Therapy Methods & Clinical Development 19, p. 362-373).

AAV capsids can be captured by placing the bead-lysate emulsion on an end-over-end mixer at 20 RPM for 30 min. at room temperature. The emulsion of beads can then be resolved by centrifuging. The supernatant can be removed and the beads washed with an appropriate buffer, with centrifugation to pellet the beads. The AAV particles can then be eluted by addition of Glycine pH 2.5, 0.01% PF-68 before recovery of the acidic glycine fraction and neutralization by addition of Tris, pH 9.0, 1.5M NaCl.

In some embodiments, recombinant virus particles described herein can be harvested from packaging cells and purified by methods standard in the art (e.g., Clark et al, Hum. Gene Ther., 10 (6): 1031-1039 (1999); Schenpp and Clark, Methods Mol. Med., 69 427-443 (2002); U.S. Pat. No. 6,566,118 and WO 98/09657, incorporated herein in their entirety by reference) such as by cesium chloride ultracentrifugation gradient or column chromatography. In one embodiment, a harvesting or purification step provided in U.S. Patent Application Publication Nos. 2021/0317474 and/or 2021/0079422 is used to harvest or purify one of the recombinant virus particles described herein. The contents of U.S. Patent Application Publication Nos. 2021/0317474 and 2021/0079422 are incorporated herein in their entirety for all purposes.

After a suitable cell expansion period post-transformation, in some embodiments of a purification method, the Nannochloropsis cells are lysed and the recombinant viral particles harvested.

In one embodiment, Nannochloropsis can be lysed with a hydrophilic ionic liquid in a lysing reactor. See, for example, U.S. Pat. No. 8,211,307, the content of which are incorporated by reference herein in its entirety.

In some embodiments, the cells are dissociated from the reactor before the cell lysis process is initiated. In some embodiments, the cells are lysed in situ. Optionally, the viral particles are harvested without lysing. In some embodiments, an endonuclease is added, e.g., circulated into the bioreactor to a final target concentration. The endonuclease may be one that degrades both DNA and RNA. Endonucleases that are produced under cGMP conditions can be used in the purification methods disclosed herein. In one embodiment, benzonase is added to the bioreactor to a final concentration of between 50-200 U/ml, e.g., 75-150 U/ml, e.g., about 100 U/mL. In some embodiments the addition of Benzonase significantly reduces host cell DNA while allowing for high vector genome production in a bioreactor.

In some embodiments, the endonuclease is allowed to mix before the lysis buffer is added to the reactor. In some embodiments, the cell lysis solution is allowed to mix with the adherent cells for up to 1 hour, up to 2 hours, up to 3 hours, up to 4 hours or up to 5 hours. In some embodiments, the lysis buffer may comprise magnesium chloride and/or Tween-20 in a suitable buffer. In an exemplary embodiment, the lysis buffer is 500 mM HEPES, 10% Tween 20, 20 mM MgCl₂, pH 8.0. A salt sucrose solution (SSS) which quenches the Benzonase reaction may be added to stop the lysis reaction. In some embodiments, the SSS is added to a harvest bag comprising rinse buffer and mixed for 15 minutes. In some embodiments, the bioreactor is rinsed with a Bioreactor Rinse Buffer, and the rinse is then collected in the harvest collection bag, along with the quenched cell lysis solution and the lysed cell contents, all of which together comprises the bulk harvest. In some embodiments, the Bioreactor Rinse Buffer may comprise Tris, MgCl₂, NaCl, Tween-20 and sucrose. In an exemplary embodiment, the Bioreactor Rinse Buffer comprises 20 mM Tris, 1 mM MgCl₂, 500 mM NaCl, 1% Tween-20 w/v and 1% sucrose w/v at pH 8.1.

After harvest, the bulk harvest viral particles may be concentrated and purified, typically via filtration. In one embodiment, the viral particles are filtered by depth filtration followed by filtration through a filter that removes large molecule contaminants and cell debris, for example a 0.45 μm filter, but that permits vector genomes to pass therethrough. Any suitable depth filter may be used. As understood in the art, depth filtration refers to the use of a porous filter medium to clarify solutions containing significant quantities of large particles (e.g., intact cells or cellular debris) in comparison to membrane filtration which would rapidly become clogged under such conditions. A variety of depth filtration media of varying pore sizes are commercially available from a variety of manufacturers such as Millipore, Pall, General Electric, and Sartorious.

In one embodiment, tangential flow filtration is used to concentrate the bulk harvest, and remove salts and proteins. TFF (also referred to as cross flow filtration (CFF)) is well known to those of skill in the art and equipment and protocols for its implementation in a wide range of situations are commercially available from a variety of manufacturers. Generally, TFF may involve the recirculation of the retentate across the surface of the membrane. This gentle cross flow feed can, in certain embodiments, minimize membrane fouling, maintain a high filtration rate, and provide high product recovery. In one embodiment, the TFF step may be implemented with a flat sheet system. Flat sheet systems may be used in large scale production where such systems are provided with a means (e.g., an open flow channel) to prevent excessive shear forces on the viral particles. Alternatively, the TFF step may be implemented with a hollow fiber system. In one embodiment, the Molecular Weight Cut Off (MWCO) of the TFF system is between 200-400 kDa, e.g., about 300 kDa.

Once all bulk harvest has been filtered, the depth filter may, in certain embodiments, be chased with the diafiltration buffer used for a subsequent first tangential flow filtration step (“TFF1”). The depth filter pool is mixed. The depth filter pool may then be filtered through a, e.g., 0.45 μm filter to further clarify the bulk harvest material. The 0.45 μm filter is then chased with TFF1 buffer.

In one embodiment, the TFF1 step is performed using a 300 kDa MW cut-off regenerated cellulose membrane cassette. The cassette is flushed and sanitized with NaOH solution and equilibrated with TFF1 buffer. In one embodiment, the TFF1 buffer comprises 20 mM Tris, 1 mM MgCl₂, 500 mM NaCl, 1% Sucrose, pH 8.1.

In some embodiments, the concentration phase of the TFF1 step is selected to reduce the volume of the clarified harvest approximately 10×. Once the target retentate volume is reached, diafiltration operations may be started. The retentate can, in some embodiments, be diafiltered with about 6 diavolumes of TFF1 buffer. In some embodiments, the retentate is diafiltered with about 5-20, or 10-15, or 12 diavolumes of TFF1 buffer. Once 6 diavolumes of permeate total flow have been achieved, the retentate may be concentrated again and harvested. Rinses, e.g., two successive rinses of the membrane, may be executed to increase the product recovery of an intermediate drug substance.

In one embodiment, the intermediate drug substance may be frozen on dry ice or in a freezer and then transferred to ≤−60° C. storage. In one embodiment, the intermediate product is not frozen prior to the downstream process. In some embodiments, multiple intermediate product substance lots are pooled together for further processing (e.g., for purification by a downstream process, e.g., as described herein). The multiple intermediate product substance lots may be pooled prior to freezing and storage. In one embodiment, the multiple intermediate product substance lots are pooled after thawing the frozen and stored lots.

In some embodiments, a downstream process is used to process the intermediate product (e.g., the pooled intermediate product) to a filtered drug substance. In some embodiments, the downstream process steps include: (a) acidification and clarification (e.g., using filtration), (b) cation exchange chromatography, (c) tangential flow filtration (“TFF2”), (d) CsCl ultracentrifugation, (e) collection of viral vector and (f) further tangential flow filtration (“TFF3”) to produce a filtered drug substance where the purified AAV particles are suspended in a pharmaceutically acceptable carrier. In some embodiments, the downstream process contains one or more of the following manufacturing steps subsequent to production of the TFF1 intermediate: thaw and pool TFF1 intermediate, acidification and clarification, cation exchange chromatography (CEX), tangential flow filtration (TFF2), CsCl ultracentrifugation for full/empty capsid separation, tangential flow filtration (TFF3) for concentration/buffer exchange, dilution and filtration of drug substance to produce drug product, storage of the drug product and filling of drug product into vials.

In embodiments where the intermediate is frozen, the downstream process begins by thawing the TFF1 intermediate material. A detergent, e.g., Tween20, may be used to promote flocculation of the bulk of host cell proteins and DNA under acidic pH. The pH of the TFF1 intermediate containing detergent may then be lowered. The flocculant and precipitate formed when the pH is lowered may then be removed by filtering the solution through a depth filter and a filter that removes large molecule contaminants and cell debris, for example a 0.45 μm filter, but that permits vector genomes to pass therethrough. Any suitable depth filter may be used.

In one embodiment, Tween20 is slowly added to the TFF1 Intermediate solution to achieve final concentration of between 10-20% Tween 20. In some embodiments, the target composition after addition of Tween 20 is 36% Tween 20 solution in 20 mM Tris, 1 mM MgCl₂, 500 mM NaCl, 1% Sucrose m/v, pH 8.1. In some embodiments, Tween20 is added slowly over a span of about 1-6 hours. In some embodiments, Tween20 is added slowly over 3-6 hours. In some embodiments, Tween20 is added slowly over 4 hours. In some embodiments, the Tween 20/TFF1 Intermediate solution is allowed to incubate overnight at room temperature. In some embodiment, the Tween 20/TFF1 Intermediate solution is allowed to incubate for 8-20 hours at room temperature. In an exemplary embodiment, the Tween 20/TFF1 Intermediate solution is allowed to incubate for 12-20 hours at room temperature.

After incubation the pH of the Tween 20 containing TFF1 Intermediate may optionally be lowered by adding any suitable acid. In some embodiments, 1M glycine pH 2.5 is added to achieve a target pH of 3.5±0.1. In some embodiments, the target pH is pH 3.0-4.0, about pH 3.3-3.7, about pH 3.4-3.6, or about pH 3.5. Once the pH is within the acceptable range, the solution may be passed through any size filter. In an exemplary embodiment, a depth filter (e.g., Clarisolve POD) in line with a 0.45 μm filter (e.g., Opticap XL10 Durapore filter) or 0.8/0.45 μm PES filter is used.

In various embodiments, a CEX capture chromatography step is used, e.g., to separate viral capsids from host cell proteins, host cell DNA, host cell lipids, Tween20 and other process-related impurities. The principles of cation exchange chromatography are well known in the art, but, briefly, this method relies on the charge-charge interactions between the positively-charged particles to be isolated and the negatively-charged resin used. In general, the column is first equilibrated by running a few diavolumes of buffer through until pH and conductivity is stabilized. The sample is then loaded and the column is washed with a loading buffer. Finally, an elution buffer is used to elute the sample of interest off the column, and fractions containing the sample are collected. The presence of the sample of interest can be detected by optical absorbance measurements of the eluant. The CEX eluate may be collected according to routine procedures and may be collected in two fractions. In one embodiment, the first fraction starts at the sharp rise in OD280 and is collected for 1.5 collection volumes (CVs). In another embodiment, the second fraction starts immediately after the first fraction and is collected for 1.0 CV. The two fractions are pooled and then neutralized to pH 8.0±0.30. In one embodiment, a Neutralization Buffer comprises 1.0 M Tris pH 9.1±0.1 at 20° C.

Cation exchange chromatography functions to separate the recombinant AAV particles from cellular and other components present in the clarified lysate and/or column eluate from the size exclusion chromatography. Examples of strong cation exchange resins capable of binding rAAV particles over a wide pH range include, without limitation, any sulfonic acid based resins as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, Mass.). Additional examples include Capto S, Capto S ImpAct, Capto S ImpRes (strong cation exchangers available from GE Healthcare, Marlborough, Mass.), and commercial DOWEX®, AMBERLITE®, and AMBERLYST® families of resins available from Aldrich Chemical Company (Milwaukee, Wis.). Weak cation exchange resins include, without limitation any carboxylic acid based resins. Exemplary cation exchange resins also include carboxymethyl (CM), phospho (based on the phosphate functional group), methyl sulfonate(S) and sulfopropyl (SP) resins.

In some embodiments, a tangential flow filtration step is used to concentrate, remove protein impurities, and exchange the buffer to an appropriate buffer for the subsequent CsCl ultracentrifugation step. Any suitable TFF membrane may be used. In an embodiment, the TFF step utilizes 300 kD MWCO regenerated cellulose membranes.

In some embodiments, the concentration phase of this step is designed to reduce the volume of the CEX eluate. In one embodiment, the retentate is diluted 2-fold with a diafiltration buffer and the retentate is concentrated to its initial volume. In one embodiment, the diafiltration buffer is the TFF NaCl diafiltration buffer that contains 20 mM Tris, 2 mM MgCl₂, 150 mM NaCl, 0.2% Poloxamer 188, 1% Sucrose, pH 8.1±0.1 at 20° C. In such embodiments, this process may be repeated until diafiltration with the new buffer is complete. In one embodiment, the retentate is diluted 2-fold with a CsCl-containing diafiltration buffer and the retentate is concentrated to its initial volume. In an embodiment, the CsCl-containing diafiltration buffer is the TFF2 CsCl diafiltration buffer that contains 20 mM Tris, 2 mM MgCl₂, 3 M CsCl, 0.2% Poloxamer 188, pH 8.1±0.1 at 20° C. In such embodiments, this process may be repeated until diafiltration with the new buffer is complete. Once CsCl diafiltration is complete, the retentate may then be concentrated to a prescribed volume that is dependent on the system hold-up volume. In some embodiments, rinsing, e.g., two successive rinses of the membrane, are executed to maximize the product recovery from the TFF system.

In some embodiments where an AAV is used for in vivo gene transduction, the final product of rAAV may contain minimum impurities and empty particles. Two methods for purifying AAV vector from empty capsids are ultracentrifugation using either an iodixanol gradient or a CsCl gradient, each of which can be employed herein.

In some embodiments, an ultracentrifugation step is used, e.g., to separate empty capsids from full capsids. Ultracentrifugation may be performed by analytical ultracentrifugation, and may involve the use of gradient buffers. Examples of gradient buffers include but are not limited to CsCl, sucrose, iodixanol and others known in the art. Centrifugation can be performed in any centrifuge capable of reaching the desired g-forces, e.g., an automated Optima XPN 100 Ultra Centrifuge system or equivalent system equipped with Type 50.2 Ti rotor or equivalent rotor. After ultracentrifugation, empty capsids and full capsids separate into different bands within the tube, and may be extracted by drawing material from a specific band. In some embodiments, TFF2-purified filtered material is centrifuged at 241,600-302,000×g (~40,000-50,000 rpm in 50.2 Ti rotor). In some embodiments, TFF2-purified filtered material is centrifuged overnight. In some embodiments, TFF2-purified filtered material is centrifuged for 16-24 hours. In some embodiments, TFF2-purified filtered material is centrifuged for 20-24 hours. In some embodiments, TFF2-purified filtered material is centrifuged at 15-25° C. In an embodiment, TFF2-purified filtered material is centrifuged at 302,000×g (50,000 rpm in 50.2 Ti rotor) for 17 hours at 20° C. In some embodiments, the buffer for CsCl centrifugation can have one or more of the following ingredients, comprising (a) CsCl, further comprising one or more of (b) MgCl₂, (c) Poloxamer 188 and (d) Tris. In some embodiments, the buffer for CsCl can include all of (a), (b), (c) and (d). In some embodiments, the buffer for CsCl has a pH 7.5-8.5, or pH 7.9-8.2. In an embodiment, a suitable buffer for CsCl centrifugation is 20 mM Tris, 2 mM MgCl₂, 3 M CsCl, 0.2% Poloxamer 188, pH 8.1±0.10. After completion of the centrifugation step, tubes may be removed from the ultracentrifuge. In some embodiment, the highest band, Band A, contains the empty capsids. In some embodiments, the next highest bands, Bands B, C and D, contain the full capsid doublet bands. In some embodiments, the AAV viral vectors are collected using a syringe. In an embodiment, Bands B, C and D are removed by an 18 G needle attached to 30 ml syringe inserted just below band D to middle of tube. In other embodiments, the bands may be assayed for the presence of full or empty capsid using techniques known in the art and/or as described herein, and the bands containing full capsid collected.

The ratio of empty to non-empty viral capsids can be measured by standard laboratory techniques. In some embodiments, the measurement is done by optical absorbance measurements. In some embodiments, the measurement is done by UV absorbance measurements. In some embodiments, the total amount of capsid proteins and total amount of DNA can be determined from UV absorbance measurements. In some embodiments, the measurement is done by optical refractive index measurements. In some other embodiments, the measurement is done by analytical ultracentrifugation.

In one embodiment, the AAV viral vector collected after ultracentrifugation has <about 15% empty capsids, <about 10% empty capsids, <about 8% empty capsids, <about 7% empty capsids, <about 5% empty capsids, <about 3% empty capsids, or <about 1% empty capsids. In one embodiment, the AAV viral vector collected after ultracentrifugation has about 1-10% empty capsids. In one embodiment, the AAV viral vector collected after ultracentrifugation has about 2-8% empty capsids. In one embodiment, the number of empty capsids is below the limit of detection. In another embodiment, the percentage of empty capsids is determined as a percentage of total capsids.

In some embodiments, a tangential flow filtration step is used to remove CsCl and concentrate the full vector capsids. Tangential flow filtration may be performed using suitable membranes. In one embodiment, 300 kDa MWCO regenerated cellulose membranes are used. The vector capsids may be retained by the membranes. The concentration phase of TFF operation may be designed to reduce the concentration of residual CsCl and volume of the ultracentrifugation pool. In some embodiments, once the target retentate volume is reached, diafiltration is started. The retentate is diafiltered with up to 10 diavolumes of a suitable TFF buffer. In one embodiment a suitable TFF buffer can include one or more of the following components, (a) Tris, (b) MgCl₂, (c) NaCl, or (d) Poloxamer 188. In one embodiment, a suitable TFF buffer can include all of (a), (b), (c) and (d). In one embodiment, the TFF buffer has pH 7.5-8.5, pH 7.7-8.3, or pH 8.0. In an embodiment a suitable TFF3 buffer comprises 20 mM Tris, 1 mM MgCl₂, 200 mM NaCl, 0.001% Poloxamer 188, pH 8.0±0.1 at 20° C. In another embodiment, a suitable TFF buffer comprises 20 mM Tris, 1 mM MgCl₂, 200 mM NaCl, 0.005% Poloxamer 188, pH 8.0=0.1 at 20° C. In one embodiment, the concentrated retentate is filtered using a 0.2 μm Pall Supor® EKV Sterilizing-Grade Filter to produce a filtered drug substance.

The purification methods described herein, in one embodiment, comprise one or multiple chromatography steps. For example, in one embodiment of a purification method, a first and optionally a second chromatography step is employed to purify recombinant AAV particles produced by the microalgae. The first chromatography step, in one embodiment, is cation exchange chromatography or anion exchange chromatography. In one embodiment, a first chromatography step comprises cation exchange chromatography and a second chromatography step comprises either anion exchange chromatography or size exclusion chromatography (SEC). Thus, in one rAAV purification method, purification is via cation exchange chromatography, followed by purification via anion exchange chromatography. In another rAAV purification method, purification is via cation exchange chromatography, followed by purification via SEC.

In another embodiment, a first chromatography step is affinity chromatography. In a further embodiment, a second chromatography step comprising anion exchange chromatography is employed.

In one embodiment, a third chromatography step is performed subsequent to the first and second chromatography steps. In one embodiment, the third chromatography step follows cation exchange, anion exchange, size exclusion or affinity chromatography.

In one recombinant AAV particle purification method, purification comprises a first chromatography step comprising cation exchange chromatography, a second chromatography step comprising anion exchange chromatography, and a third chromatography step comprising SEC. In yet another embodiment, purification is via cation exchange chromatography (first chromatography step), followed by purification via size exclusion chromatography (SEC) (second chromatography step), followed by purification via anion exchange chromatography (third chromatography step).

In yet an additional embodiment, recombinant AAV particle purification is via affinity chromatography, followed by anion exchange chromatography, followed by SEC. In yet even another embodiment, recombinant AAV particle purification is via affinity chromatography, followed by SEC, followed by anion exchange chromatography.

Anion exchange chromatography, in one embodiment, functions to separate AAV particles from proteins, cellular and other components present in the clarified lysate and/or column eluate from the size exclusion chromatography. Anion exchange chromatography can also be used to control the amount of empty capsids in the eluate. For example, the anion exchange column having recombinant AAV particle bound thereto can be washed with NaCl at a modest concentration (e.g., about 100-125 mM, such as 110-115 mM) and a portion of the empty capsids can be eluted in the flowthrough without substantial elution of the rAAV vectors. Subsequently, rAAV vector bound to the anion exchange column can be eluted using NaCl at a higher concentration (e.g., about 130-300 mM Nacl), thereby producing a column eluate with reduced or depleted amounts of AAV empty capsids and proportionally increased amounts of rAAV.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography include without limitation, MACRO PREP Q (strong anion-exchanger available from BioRad, Hercules, Calif.); UNOSPHERE Q (strong anion-exchanger available from BioRad, Hercules, Calif.); POROS 50HQ (strong anion-exchanger available from Applied Biosystems, Foster City, Calif.); POROS XQ (strong anion-exchanger available from Applied Biosystems, Foster City, Calif.); POROS 50D (weak anion-exchanger available from Applied Biosystems, Foster City, Calif.); POROS 50PI (weak anion-exchanger available from Applied Biosystems, Foster City, Calif.); Capto Q, Capto XQ, Capto Q ImpRes, and SOURCE 30Q (strong anion-exchanger available from GE healthcare, Marlborough, Mass.); DEAE SEPHAROSE (weak anion-exchanger available from Amersham Biosciences, Piscataway, N.J.); Q SEPHAROSE (strong anion-exchanger available from Amersham Biosciences, Piscataway, N.J.). Additional exemplary anion exchange resins include aminoethyl (AE), diethylaminoethyl (DEAE), diethylaminopropyl (DEPE) and quaternary amino ethyl (QAE).

Chromatography medium such as cation exchange, anion exchange, size exclusion and affinity can be equilibrated, washed and eluted with various buffers under various conditions such as pH, and buffer volumes.

Cation exchange chromatography may be equilibrated using standard buffers and according to the manufacturer's specifications. For example, chromatography media can be equilibrated with a phosphate buffer, at 5 to 100 mM, or 10-50 mM, such as 10-30 mM, and sodium chloride. After equilibration, sample is then loaded. Subsequently, the chromatography media is washed at least once, or more, e.g., 2-10 times. Elution from the chromatography media is by way of a high salt buffer, at least once, but elution may be 2 or more times with the same or a higher salt buffer.

Typical equilibration buffers and solutions for washes and elutions for cation exchange chromatography are at an appropriate pH, of from about pH 3 to pH 8, more typically from about pH 4 to pH 7.5, such as pH 6.0-6.5, 6.5-7.0, 7.0-7.5, or any pH at or between the stated ranges such as, 7.0, 7.1, 7.2, 7.3 or 7.4.

Appropriate equilibration buffers and solutions for washes and elutions for cation exchange columns are known in the art and are generally anionic. Such buffers include, without limitation, buffers with the following buffer ions: phosphate, acetate, citrate, borate, or sulfate.

In one embodiment, the cation exchange chromatography media is first equilibrated, sample applied, and washed with a low salt concentration, e.g., 10-150 mM of NaCl, such as 10, 20, 25, 30, 35, 40, 45, 50, 55, 60, 60-125 mM, or any concentration at or within these ranges, such as, 100 mM. Following a first wash, the chromatography media may be treated with a higher salt concentration in order to elute impurities, such as a higher NaCl concentration, or with another buffer with a greater ionic strength. After additional impurities are eluted from the column, to elute recombinant AAV particles, the ionic strength of the buffer may be increased using a salt, such as NaCl, KCl, sulfate, formate or acetate, and recovered. In one embodiment, elution is with a high salt concentration, e.g., 200-500 mM of NaCl, or any concentration at or within these ranges, such as 250 mM, 300 mM, 350 mM, or 400 mM.

Additional components can be included in the equilibration buffers and solutions for washes and elutions. For example, a wash buffer for cation exchange chromatography can include an anionic surfactant such as sarkosyl (e.g., 1-10 mM), a wash buffer for anion exchange chromatography can include a cationic surfactant such as dodecyltrimethylammonium chloride (e.g., 1-10 mM).

Typical equilibration buffers and solutions for washes and elutions for anion exchange chromatography an appropriate at a pH of from about pH 7.5 to pH 12, more typically from about pH 8.0 to pH 10, and even more typically from about pH 8.0 to pH 9.0, such as pH 8.1, 8.2, 8.3, 8.4, 8.5, 8.6, 8.7, 8.8, 8.9 or 9.0. Appropriate equilibration buffers and solutions for washes and elutions for anion exchange columns are generally cationic or zwitterionic in nature. Such buffers include, without limitation, buffers with the following buffer agents: N-methylpiperazine; piperazine; Bis-Tris; Bis-Tris propane; Triethanolamine; Tris; N-methyldiethanolamine; 1,3-diaminopropane; ethanolamine; acetic acid, and the like. To elute the sample, the ionic strength of the starting buffer is increased using a salt, such as NaCl, KCl, sulfate, formate or acetate. Such equilibration buffers and solutions for washes and elutions can have the foregoing buffering agents from about 5-100 mM, more typically from about 10-50 mM.

In one embodiment, the anion exchange chromatography media is first equilibrated, sample applied, and washed with a low salt concentration, e.g., 50-150 mM of NaCl, such as 50-60, 60-70, 70-80, 80-90, 90-100, 100-100 mM, or any concentration at or within these ranges. Following a first wash, the chromatography media may be treated with a higher salt concentration in order to elute impurities such as empty capsids, such as a higher NaCl concentration, or with another buffer with a greater ionic strength. One example for use as the second buffer is a Tris-based buffer with a NaCl concentration of about 110 mM-125 mM, or any concentration at or within these stated ranges. After additional impurities are eluted from the column, the recombinant AAV particles can be recovered by elution with a higher concentration of salt. One example for an elution buffer is a Tris-based buffer with a NaCl concentration of 125 mM or greater, such as 125-150 mM, 150-200 mM or 200-250 MM NaCl, or any concentration at or within these stated ranges.

In the anion exchange chromatography media wash solutions, polyethylene glycol (PEG) may be included. This is referred to as polyethylene glycol (PEG) modulated column chromatography. PEG wash solutions can be applied to the anion exchange chromatography media prior to elution of AAV vector particles. Typically PEG in such wash solutions has an average molecular weight in a range of about 1,000 to 80,000 g/mol, inclusive. Typical amounts of PEG in such wash solutions range from about 0.1% to about 20% PEG or any amount at or within these stated ranges, or from about 1% to about 10% PEG or any amount at or within these stated ranges.

Size-exclusion chromatography (SEC) media may be equilibrated using standard buffers and according to the manufacturer's specifications. For example, chromatography media can be equilibrated with a phosphate buffer, for example, at about 1-5 mM, 5-50 mM, or 5-25 mM, and NaCl, for example, at about 50-100 mM, 100-150 mM, 150-200 mM, 200-250 mM, 250-300 mM, or 300-400 mM, or any amount at or within these stated ranges. After equilibration, sample is then loaded. Subsequently, the flow through containing the rAAV particles is recovered. Additional volumes of buffer (e.g., phosphate buffer), based upon the amount of chromatography media and/or column size, can be added for rAAV particle recovery.

In some embodiments, size exclusion chromatography media has a separation range (molecular weight) between about 10,000 and 600,000, inclusive. Particular resins (media) appropriate for size exclusion chromatography include without limitation particles or beads of porous cellulose, crosslinked agarose, crosslinked dextran, styrene-divinylbenzene (Dianon HP-20), polyacrylamide (Bio Gel), methacrylic (Toyopearl), and controlled pore glass.

Affinity columns are typically composed of a ligand linked or conjugated to a substrate. Particular examples of ligands include AAV binding antibodies. Such substrates include sepharose and other materials typically used in such affinity purification applications and can be made or are commercially available (e.g., AVB Sepharose™ High Performance, GE Healthcare, Marlborough, Mass.).

Appropriate equilibration buffers and solutions for washes and elutions for affinity columns are typically Tris or acetate based. For example, affinity chromatography media can be equilibrated with a Tris buffer, for example, at about 1-5 mM, 5-50 mM, or 5-20 mM, and NaCl, for example, at about 50-100 mM, 100-150 mM, 150-200 mM, 200-250 mM, 250-300 mM, or any amount at or within these stated ranges. Typical equilibration buffers for affinity chromatography is a pH of from about pH 7.5 to pH 9.0, more typically from about pH 8.0 to pH 8.5, and even more typically a pH such as pH 8.0, 8.1, 8.2, 8.3, 8.4, or 8.5.

After equilibration, sample is then loaded. Subsequently, the rAAV particles are eluted from the for affinity column by reducing pH of the buffer to less than 7.0. Elution buffers may be acetate based and typically pH is less than 5.0, more typically less than 4.0, such as less than 3.0, more specifically between about 2.0 and 3.0, or any pH at or within these stated ranges.

Volumes of buffer for equilibration, washing and elution can be based upon the amount of chromatography media and/or column size to achieve rAAV particle recovery. Typical volumes are 1-10 column volumes.

Column eluate is/are collected following the elution(s)/flow through from each of the chromatography steps. AAV can be detected in the fractions using standard techniques, such as monitoring UV absorption at 260 and 280 nm.

Eluates comprising rAAV particles from any of the cation exchange, anion exchange, size exclusion, and/or affinity chromatography steps as described herein can, if desired, be efficiently concentrated by ultrafiltration/diafiltration. Reduction in volume can be controlled by the skilled artisan. In particular non-limiting examples the reduction in volume achieved is between abut 1-30 fold, inclusive. Thus, a 1-fold reduction reduces the volume by half, e.g., 1000 ml is concentrated to 500 mL. A 10 fold reduction reduces the volume by a factor of 10, e.g., 2000 ml is concentrated to 200 mL. A 20 fold reduction reduces the volume by a factor of 20, e.g., 2000 ml is concentrated to 100 mL. A 30 fold reduction reduces the volume by a factor of 30, e.g., 2000 ml is concentrated to 66.67 mL.

A non-limiting example of ultrafiltration/diafiltration is tangential flow filtration (TFF). For example, a hollow fiber membrane with a nominal pore size corresponding to a 100 kDa molecular weight cutoff, so that large amounts of AAV vector can be prepared when present in larger volumes of eluate.

### EXAMPLES

The present disclosure is further illustrated by reference to the following Examples. However, it should be noted that these Examples, like the embodiments described above, are illustrative and are not to be construed as restricting the scope of the present disclosure in any way.

### Example 1—Production of AAV Proteins by Nannochloropsis oceanica

### Methods

### Vector Design

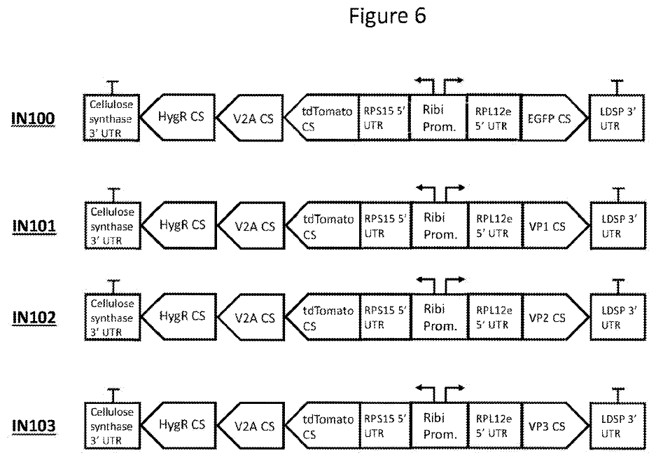

A vector backbone based on the endogenous N. oceanica genetic elements found in pNOC-CRISPR-ARS from Poliner et al. 2018. was designed de novo (Poliner et al. (2018). ACS Synth. Biol. 7, pp. 962-968, incorporated by reference herein in its entirety for all purposes). Additional restriction enzyme sites flanking the algae-specific payload were included, and some restriction motifs included in the 5′ and 3′ UTRs in pNOC-CRISPR-ARS that were unnecessary for DNA assembly methods were removed. The entirety of the vector was synthesized by Genscript (Piscataway, New Jersey) in a minimal pUC57 backbone containing an EGFP coding sequence between the Ribi bidirectional promoter (SEQ ID NO:1) and the LDSP 3′UTR (SEQ ID NO:36) instead of a Cas9 coding sequence found in pNOC-CRISPR-ARS. See FIG. 6, IN100.

Expression from one side of the Ribi promoter has been reported to be well correlated with expression from the other side of the promoter (Poliner et al. (2020). Algal Research 45:101664, incorporated by reference herein in its entirety for all purposes). As such, to detect high expressing clonally derived integrants, a tdTomato fluorescence reporter coding sequence (SEQ ID NO:71) that is transcriptionally and translationally linked to a Hygromycin cassette by a 2A ribosome skip sequence (SEQ ID NO: 12) was placed between the opposite side of the Ribi promoter and the cellulose synthase 3′ UTR (SEQ ID NO:39), instead of the sgRNA cassette found in pNOC-CRISPR-ARS. The ARS and CEN regions of pNOC-CRISPR-ARS were not included because all vectors were integrated into the host genome. This expression cassette is referred to herein as IN100, and the vector containing the expression cassette as pIN100.

From the pIN100 vector, to make pIN101, the native AAV9 VP1 coding sequence (SEQ ID NO:31) was subcloned in place of eGFP sequence using a standard PCR-based fragment generation and Gibson-Assembly approaches with Hot-Start Q5 DNA polymerase and the NEB Builder HiFi assembly kit (New England Biolabs, Ipswich, Massachusetts). Similarly, IN102 replaced EGFP with the AAV9 VP2 coding sequence where the non-standard threonine start codon was replaced with a canonical methionine start codon (SEQ ID NO:73). The AAV9 VP3 coding sequence (SEQ ID NO:34) replaced EGFP in pIN103. FIG. 6 provides the architecture of IN100, IN101, IN102 and IN103 expression cassettes.

### Nannochloropsis Strain and Culturing

Nannochloropsis oceanica (CCMP1779) was obtained from the Bigelow National Center for Marine Algae and Microbiota stock center (East Boothbay, ME). An axenic stock was derived by successively re-streaking to single colonies on 1.5% wt/wt Agar saltwater plates supplemented with standard f/2 macro- and micro-nutrients (Guillard and Ryther (1962). Canadian Journal of Microbiology 8, pp. 229-239, incorporated by reference herein in its entirety for all purposes). Subsequent culturing was conducted in f/2 media but using 0.5× sea water salt concentration (17.98 g/L Instant Ocean saltwater mix) with the same nitrate, phosphate, and trace metal concentrations of f/2 media. The same formulation was used for liquid media. Culturing was conducted under 100-200 μE/m²/sec PAR violet, blue, and red LED arrays at room temperature (RT). Liquid cultures were grown in Erlenmeyer flasks on an orbital shake table at 125 rpm under the same lighting conditions.

### Transformation

Vectors were linearized by restriction digest of the construct using restriction sites engineered into the bacterial backbone and outside of the terminal 3′ UTRs. This was done using KpnI and HindIII (New England Biolabs). Approximately 50 μg-70 μg of vector DNA was digested in a 300 μL reaction volume in New England Biolabs Cutsmart buffer under standard conditions. Enzyme was added to achieve 5× overdigest in 3 hr. (1.6 U of enzyme per μg of plasmid DNA). The reaction was terminated by DNA precipitation. Briefly, 5 M NaCl was added to the reaction to a final concentration of 300 mM. An equal volume of isopropanol was added to the aqueous volume and the DNA was pelleted by centrifugation at 20,000 rcf for 25 minutes at 4° C. The isopropanol fraction was removed and the resulting pelleted washed four times in 70% ethanol. The final DNA was resuspended in 50-100 μL of 10 mM Tris-HCl pH 8.5 and 0.1 mM EDTA. The final concentration of the digested DNA was determined using the Qubit Broad Range dsDNA kit.

Transformation and electroporation were conducted as per Vieler et al. (Vieler et al. (2012). PLOS Genetics 8(11):e1003064, incorporated by reference herein in its entirety for all purposes). Briefly, cells were grown to mid-log phase (~4×10⁶ cells/mL) and then collected by centrifugation at 3000 rcf for 10 min. at 4° C. The cells were then washed three times with ice cold 375 mM Sorbitol with centrifugation at 3000 rcf for 5 min. at 4° C. to pellet the cells after each wash. The washed cells were then resuspended at a final density of 5×10⁸ cells/mL in 375 mM sorbitol before being mixed with 3 μg of linearized plasmid DNA and 30 μg of heat denatured salmon sperm DNA in a 2 mm ice-chilled electroporation cuvette (Thermo Fisher Scientific). The cells were then electroporated with a gap potential of 2.2 kV with a 50 μF capacitor and 600 Ohm shunt resistor (GenePulser II, Biorad). The cuvette was placed in a RT water bath for 2 minutes before the cells were resuspended in 10 mL of 0.5× saltwater f/2 media in a 50 mL conical tube. The electroporated culture was then placed on its side on an orbital shaker table at 125 RPM under 75 μE/m²/sec light for 24 hours to recover. Recovered cells were then pelleted as above and resuspended in 5 mL of 0.5× sea salt f/2 media. The cells were then spread on 1.5% Agar 0.5× sea salt f/2 media in petri dishes (200 μL/10 cm diameter petri dish) containing 100 μg/mL hygromycin B before growth to single colonies over 2-3 weeks under 100 μE/m²/sec intensity grow lights.

### Isolation and Characterization of Transformants

Individual colonies were picked in to 96-well microtiter plates containing 0.5× sea salt f/2 media. A row (12 wells) of wild-type (untransformed) algae was used as a control. After 3-5 days growth, the cultures were diluted into fresh media at a 1:10 dilution and grown for a further 4 days.

The plates were then assayed with a SpectraMax i3x reading well fluorescence at 440 nm excitation and 680 nm emission for chlorophyll autofluorescence as a metric of culture density. For IN100, transformants' GFP fluorescence was measured at 488 nm excitation and 518 nm emission. For all constructs, tdTomato was measured at 518 excitation and 554 nm emission. Arbitrary fluorescence intensity units from the tdTomato co-reporter were divided by the arbitrary fluorescence units of the chlorophyll autofluorescence. This ratio was compared to the mean ratio of the 12 wells of untransformed controls. Transformed clones with ~1.5× fold or greater normalized tdTomato fluorescence compared to the average of the untransformed control were then spotted on agar plates containing 100 μg/mL hygromycin B and struck down to single colonies. Single colonies were then grown in 10 mL of 0.5× saltwater f/2 media to late log phase (~1×10⁷ cells/mL) and the culture pelleted by centrifugation at 4000 rcf at RT. The cell pellet was then snap frozen and processed for western blotting.

### Western Blotting

Frozen cell pellets were resuspended in 600 μL of Bugbuster (EMDMillipore) and transferred to Lysing Matrix E bead beating tubes (2 mL tubes, MP Bio) before being disrupted in a Qiagen Tissuelyser II by three cycles of 30 seconds at 30 Herz milling with one min. of cool down time at room temperature. Cell debris was pelleted by centrifugation at 16×10³ rcf at 4° C. for 5 min, and the supernatant was recovered as the soluble protein fraction. The soluble protein fraction was mixed 1:3 with 4× Invitrogen NuPAGE LDS Sample Buffer containing 50 mM DTT. The protein was then denatured at 80° C. for 10 min. in a water bath and cooled to room temperature before loading and SDS-PAGE separation on a NuPage 8% polyacrylamide gel with MOPS SDS running buffer (ThermoFisher) at 200 V for 20-30 min. The separated protein was then transferred to a 0.22 μm pore nitrocellulose membrane via semi-dry transfer (20 V×40 min.). Membranes were washed 3×2 minutes with TBSMT (50 mM TrisHCl pH 7.4, 150 mM NaCl, 0.05% Tween-20) then blocked for 20 min. in Haycock's blocking solution; 1% wt/vol polyvinylpyrrolidone in TBSMT (Haycock (1993). Anal Biochem 208, pp. 397-399, incorporated by reference herein in its entirety for all purposes).

Recombinant protein was detected with goat anti-GFP conjugated to alkaline phosphatase (ab6661 Abcam, Cambridge, Massachusetts) at 1:5000 dilution or 1:1000 rabbit polyclonal anti-VP1/2/3 (Progen, Heidelberg, Germany, cat #61084), in Haycock blocking solution. The latter was then detected with goat anti-Rabbit: AP at 1:10,000 dilution (ab6722, Abcam). All antibodies were incubated for 60 min. at room temperature and 4×3 min. washes in TBSMT were conducted between steps.

Chromogenic development was performed by 1-Step BCIP/NBT reaction (Thermofisher, cat #34042) before quenching the reaction by washing in distilled water followed by TBSMT. Western blots were imaged using an Amersham ImageQuant 800 system.

### Results

FIG. 6 provides the general design of the expression cassette IN100. The N. oceanica-specific payload was separated from the bacterial origin of replication and antibiotic resistance cassette by restriction digest of the vector. The linearized DNA was then electroporated into N. oceanica and spread on hygromycin containing salt-water f/2 media agar plates until single colonies appeared ~3 weeks later under 24 hr. grow lights at RT.

Individual colonies were picked into 96-well microtiter plates and tdTomato fluorescence was read and compared to wild-type controls with chlorophyll autofluorescence being used to normalize for culture density. The cells from the top 9 expressing tdTomato wells (out of ~200 picked colonies) were then struck down to single colonies on hygromycin agar plates and re-isolated axenic clones grown to mid-log phase as liquid shake cultures.

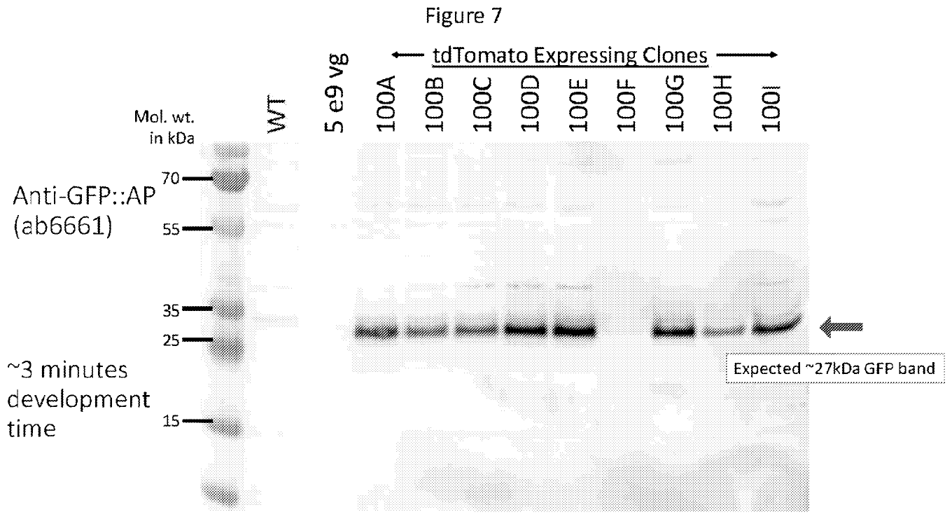

The cultures were then pelleted, lysed, and assayed for GFP expression by SDS-PAGE and western blotting using anti-GFP antibodies. Eight out of nine assayed colonies displayed strong anti-GFP immunoreactivity at the expected MW (~28 kDa) of EGFP. FIG. 7. Even the weakest expressor shows some immunoreactivity compared to the un-transformed control. This collectively demonstrates that strong target transgene expressors can be generated to rapidly identify high expressing random integration transformants.

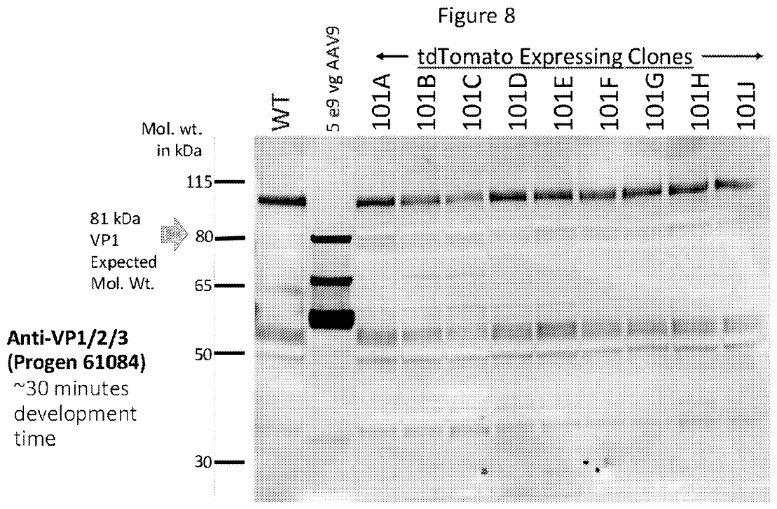

N. oceanica cells were transformed, selected and lysed as above with IN101 which drives AAV9 VP1 in place of the EGFP found in IN100. FIG. 6. The lysates were probed with a rabbit polyclonal antibody that recognizes AAV9 VP1, VP2, and VP3. Collectively, anti-VP immunoreactivity at the expected molecular weight of AAV9 VP1 (88 kDa) in multiple clones was found. FIG. 8.

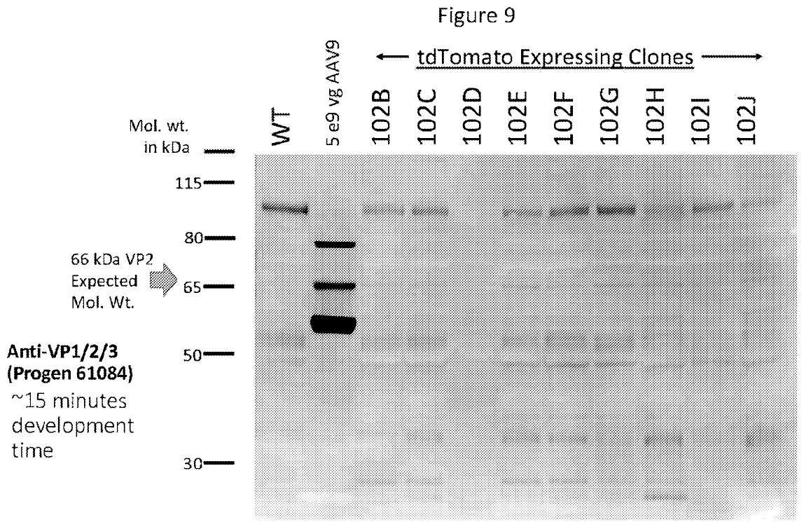

N. oceanica cells were transformed, selected and lysed as above with IN102 which drives AAV9 VP2 in place of the EGFP found in pIN100. The lysates were probed with a rabbit polyclonal antibody that recognizes AAV9 VP1, VP2, and VP3. Collectively, anti-VP immunoreactivity at the expected molecular weight of AAV9 VP2 (66 kDa) in multiple clones was found. See FIG. 9.

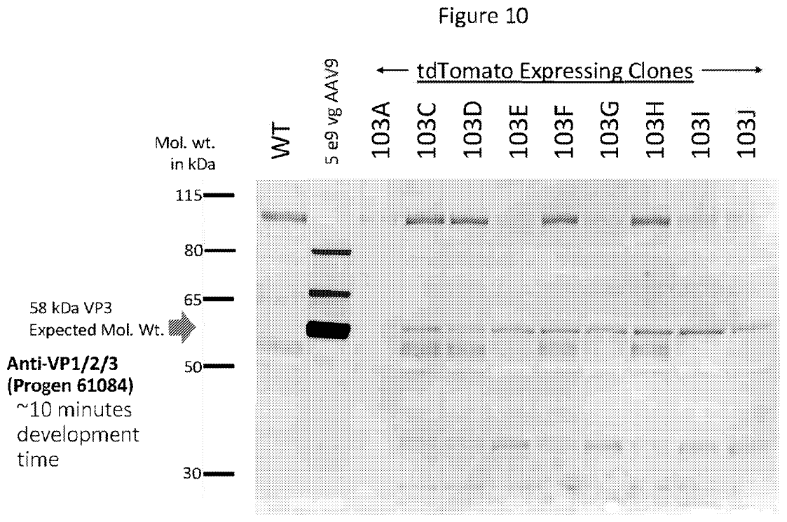

N. oceanica cells were transformed, selected and lysed as above with IN103 which drives AAV9 VP3 in place of the EGFP found in IN100. The lysates were probed with a rabbit polyclonal antibody that recognizes AAV9 VP1, VP2, and VP3. Collectively, anti-VP immunoreactivity at the expected molecular weight of AAV9 VP3 (58 kDa) in multiple clones was observed. See FIG. 10.

### Example 2—Production of AAV Capsids by Nannochloropsis oceanica

### Generation of Ribosomal-Locus Docking Site

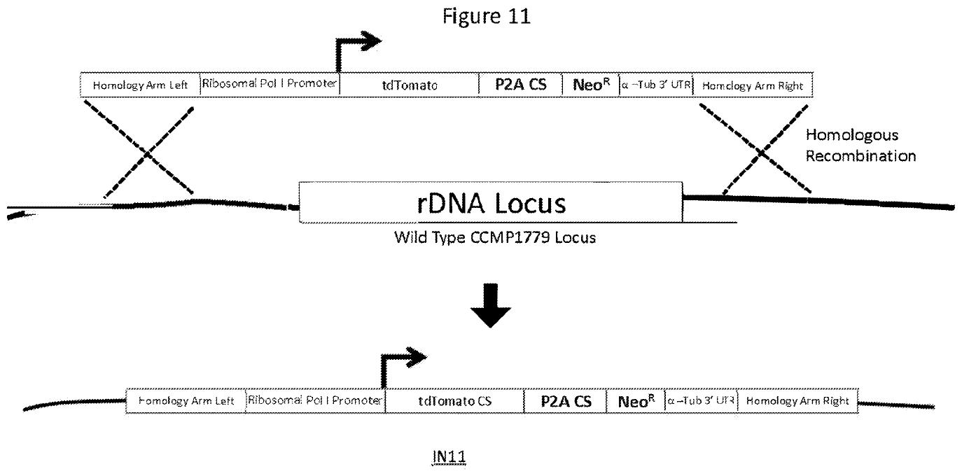

The ribosomal locus docking-site design vector LP-tdTomato in Sudfeld (Sudfeld et al. (2022). Molecular Plant 15, pp. 340-353) was modified to use a Tn5-transposon derived Neomycin resistance cassette, rather than a Zeocin resistance cassette (See FIG. 11 for a drawing of the expression cassette). The Tn5-derived Neomycin resistance (NeoR) transgene enzyme also inactivates Kanamycin and G418, the latter of which has been used to select integrants. The initial rDNA docking-site was generated by taking a wild-type Nannochloropsis oceanica (strain CCMP1779) and electroporating the linearizing docking-site cassette (also referred to herein as IN11; See FIG. 11).

Nucleic acid sequences of the various portions of the IN11 expression cassette are as follows:
Homology arm left: SEQ ID NO:66
Homology arm right: SEQ ID NO:67
Pol I Promoter: SEQ ID NO:49
Td Tomato coding sequence: SEQ ID NO:71
P2A peptide coding sequence: SEQ ID NO:72
NeoR coding sequence: SEQ ID NO:51
α-tubulin 3′ UTR: SEQ ID NO:38

Integrant colonies were first selected on G418 media (100 μg/μL), picked in to 96-well microtiter plates, and then down-selected by observing tdTomato fluorescence with a brightfield fluorescence microscope using a standard rhodamine-appropriate filter set. The strongest tdTomato fluorescing colonies were confirmed to be on-target integration events by polymerase chain reaction (PCR) using a primer internal to the docking-site cassette and one external on expected 3′ flanking sequence of the ribosomal locus.

### Generation of Individual VP Expression Vectors Using the Ribosomal-Locus Docking Site

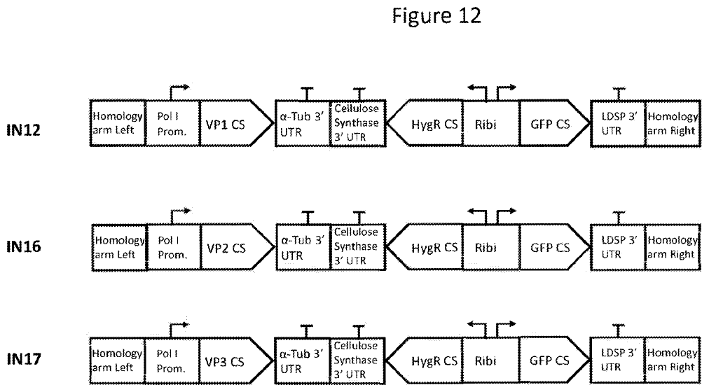

To test the expression of individual AAV9 VP proteins from the rDNA docking-site, various integration expression cassettes were developed. For these cassettes, the native AAV9 VP1 (SEQ ID NO:31), VP2 (SEQ ID NO:32), or VP3 (SEQ ID NO:34) coding sequences were placed downstream of the RNA Pol I-driven ribosomal promoter (construct IN12: VP1, IN16: VP2, IN17: VP3, see FIG. 12), and operably linked thereto. For IN16, the non-canonical ACG start codon of VP2 was replaced with the canonical ATG start codon. A second RNA Pol II-driven cassette was used to select for integrants (FIG. 12). Specifically, the bi-directional Ribi-promoter (SEQ ID NO:1) expressed hygromycin-resistance (SEQ ID NO:52) from one side of the promoter and EGFP from the other. The vectors containing the expression cassettes (IN12, IN16, IN17; See FIG. 12) were linearized by restriction digest and integrated into to the ribosomal docking site using methods described by Sudfeld (Sudfeld et al. (2022). Molecular Plant 15, pp. 340-353), except with the use of commercially produced Lachnospiracaea bacterium Cas12a (LbCas12a) complexed with two additional CRISPR RNAs (crRNAs) that targeted the Neomycin resistance transgene. Integrants were selected on hygromycin containing agar media (100 μg/mL) and picked in to 96-well microtiter plates before down selection using a fluorescence microscope to select for clones that lost tdTomato expression and gained EGFP expression. Integration was confirmed by PCR amplification using primers located in the Pol I promoter region and the 3′-flanking genome integration site followed by amplicon sequencing.

Algae were grown in 17.98 g/L artificial sea water (Instant Ocean) supplemented with 0.15 g/L sodium nitrate, 0.01 g/L NaNO₃, and 0.01 g/L NaH₂PO₄·H2O, and f/2 trace metal cocktail (Guillard and Ryther (1962). Can. J. Microbiol. 5, pp. 229-239). Cultures were expanded under ~200 μmol/m²/sec photosynthetically active radiation equivalent using Fiet electric grow lights (Model: GLP24ADJS/60W/LED) at 125 RPM on Innova 2100 orbital shaker tables. Single colonies were expanded to ~1×10⁷ cells/mL, which is late log phase.

The transformed colonies were grown in small scale cultures (~15 mL) to saturation and the cells pelleted by centrifugation (3000 relative centrifugal force (rcf)×10 min.) before removal of culture medium. The resulting cell pellet was lysed in 1× NuPage lysis buffer with dithiothreitol (DTT) (Thermofisher) by bead beating with Lysing Matrix E (MP Bio) in Qiagen Tissuelyser II (30 Hz, 4×1 min. cycles with 1 min. pause). The lysate was separated on an 8% acrylamide SDS-PAGE gel using 3-(N-morpholino) propanesulfonic acid (MOPS) buffer and probed with a mouse monoclonal anti-VP protein (Clone B1) primary antibody with detection using goat-anti-mouse alkaline phosphatase conjugated secondary antibody followed by 5-bromo-4-chloro-3-indolyl-phosphate/nitro blue tetrazolium (BCIP/NBT detection).

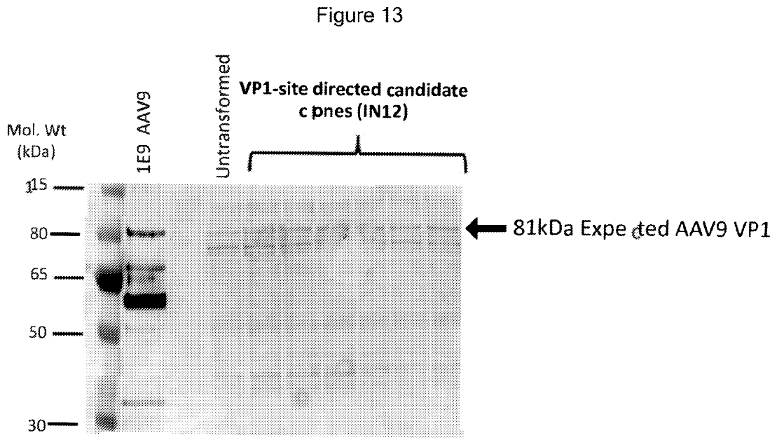

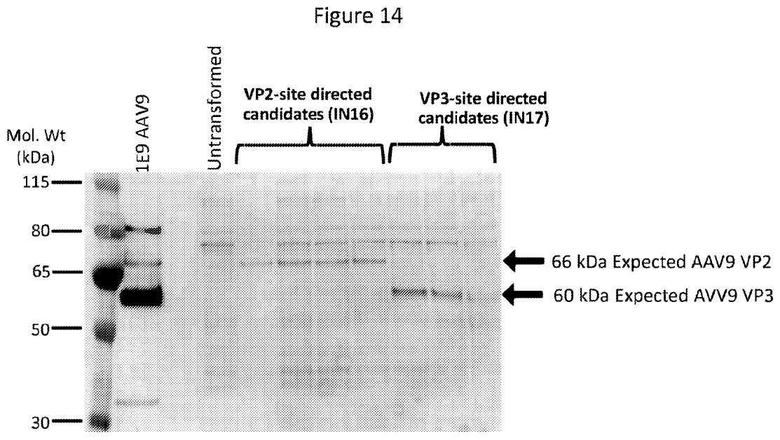

VP2 and VP3 protein expression were strongly detected in IN16 and IN17, respectively, while VP1 expression was weakly detected in IN12 (FIG. 13 (IN12), FIG. 14 (IN16, IN17)).

### Generation of a AAV9-Capsid Producing Algae Strain

Upon demonstrating successful expression of individual AAV9 VP proteins from the ribosomal locus docking-site, above, multiple variants were generated where VP1, VP2, and VP3 were expressed from either Pol I or Pol II promoters at the ribosomal locus docking site. In some cases, these cassettes contained an additional AAP-expression cassette. The AAP coding sequence was codon-optimized to avoid introduction of direct or inverted repeats and uses the canonical ATG rather than CTG start codon.

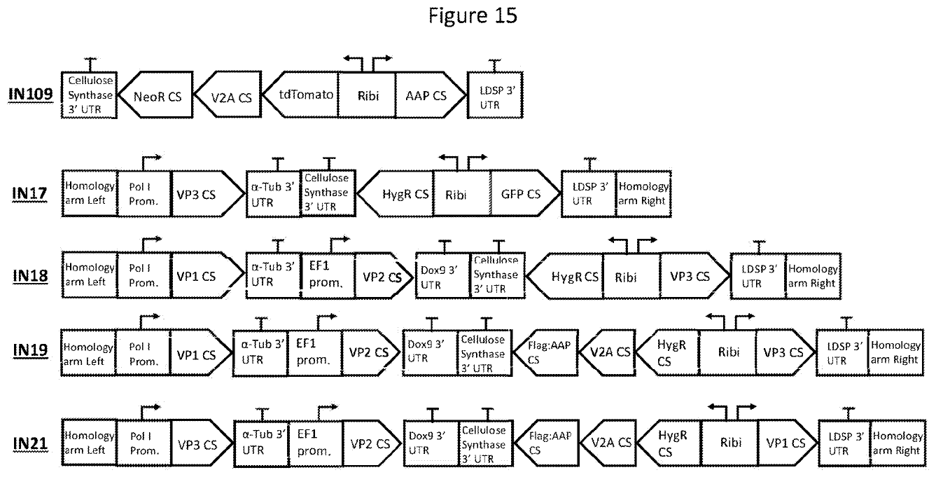

In one case, a second randomly integrated AAP-expression cassette was delivered in trans to the Pol I-driven VP3 expression construct (IN109+IN17) (See FIG. 15). Specifically, IN109 contains a Neomycin resistance cassette and a tdTomato co-reporter to detect integration in the VP3-producing algae strain.

Nucleic acid sequences used in IN109 were as follows:
Cellulose synthase 3′ UTR: SEQ ID NO:39
NeoR CS: SEQ ID NO: SEQ ID NO:51
2A peptide coding sequence: SEQ ID NO:12
tdTomato coding sequence: SEQ ID NO:71
Ribi promoter: SEQ ID NO:1
AAP coding sequence: SEQ ID NO:57
LDSP 3′ UTR: SEQ ID NO:80

In another variation “all in one” VP1 (SEQ ID NO:31), VP2 (SEQ ID NO:33), VP3 (SEQ ID NO:35) expression constructs were generated with or without AAP9 (IN19 and IN21 included AAP, while IN18 did not, see FIG. 15). AAV9 AAP was included as a downstream peptide from a viral 2A-skip peptide coding sequence (SEQ ID NO:11) placed C-terminal to the Hygromycin resistance gene (constructs IN19 and IN21, see FIG. 15). In these constructs, the native AAV9 VP1 sequence was used while VP2 (SEQ ID NO:33), VP3 (SEQ ID NO:35), and AAP (SEQ ID NO:57 or 59) were codon-optimized using the human codon usage table to ensure that direct and/or inverted repeats were not being generated in the expression cassette (to avoid recombination in both the E. coli strains used for cloning the vectors and after integration in to the Nannochloropsis genome.) Another difference here between IN21 and other strains was for the former, VP3 was driven by the strong rDNA PolI promoter (SEQ ID NO: 49), and AAP was included as a transcriptional/translational fusion to the Hygromycin resistance cassette (SEQ ID NO:52) driven by the Ribi-promoter (SEQ ID NO:1). Homology arms flank each end of the expression cassette for integration into the ribosomal DNA (rDNA) locus docking-site (SEQ ID NO:66 and 67). The IN21 expression cassette comprises the nucleic acid sequence of SEQ ID NO:77.

### Algae Transformation and Specific Description of IN21

A high expressing, PolI-driven, ribosomal-DNA locus docking site was generated as per Sudfeld (Sudfeld et al. (2022). Molecular Plant 15, pp. 340-353), with the exception of using a Neomycin/Kanamycin/G418 resistance marker rather than zeocin to select for integrants. The docking site itself was verified by PCR using primers that located in the 5′ homology arm and in the 3′ expected genomic locus outside of the 3′ homology arm used to integrate the cassette. (See FIG. 15 for diagram of the IN21 expression cassette).

The polynucleotide sequence of IN21 is provided at SEQ ID NO:77, herein. A VP1, VP2, VP3, AAP ensemble expression vector was assembled using the PolI promoter to drive a codon-optimized (human codon usage) AAV9 VP3 open reading frame (SEQ ID NO:35). The VP2 coding sequencing was codon-reoptimized (human codon usage table) (SEQ ID NO:33) to avoid directed and inverted repeats and driven using the elongation factor 1 promoter (EF1) (SEQ ID NO:50) while one side of the bi-directional (Ribi) promoter (SEQ ID NO: 1) was used to drive the native VP1 sequence (SEQ ID NO:31). A codon optimized AAP sequence (SEQ ID NO: 57) was placed 3′ to a hygromycin resistance gene coding sequence (SEQ ID NO:52) followed by a 2A ribosomal skip cassette (SEQ ID NO:12) to allow two polypeptides to be expressed from one mRNA driven by the Ribi bi-directional promoter. This expression cassette was flanked by homology arms to integrate it into the rDNA docking site locus. Cas12a-catalyzed integration was performed as described by Sudfeld (Sudfeld et al. (2022). Molecular Plant 15, pp. 340-353) with the exception of purified Cas12a and synthetized cRNAs (IDT) being supplied as and assembled ribonucleoprotein complex in trans as per manufacturer's protocol. Integration was confirmed by PCR and sequencing of the entire locus by nanopore-based sequencing (Primordium labs) on purified PCR amplicons.

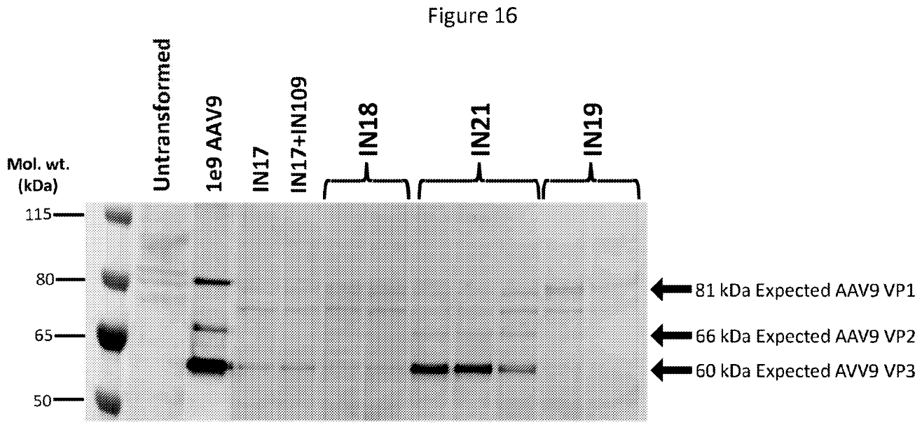

Denaturing western blot conditions were used on total cell lysate and an anti-VP1/2/3 protein monoclonal antibody (Clone B1) was employed together with goat-anti-mouse alkaline phosphatase conjugated secondary antibody followed by 5-bromo-4-chloro-3-indolyl-phosphate/nitro blue tetrazolium (BCIP/NBT detection) to detect VP protein expression. Lysates generated from equivalent cell pellet masses of multiple VP-expression vectors with or without the presence of AAP were cross compared (See FIG. 16). One expression vector, IN21, showed much stronger VP2 and VP3 expression than the other vectors examined. FIG. 16.

Isolation of AAV9 Capsids from Nannochloropsis oceanica Strain IN21

Strain IN21 cultures were grown in either 15 mL of media in upright T25 tissue culture flasks (referred to as “Small Scale” (SS) culture herein) or at 600 mL of media in 2 L baffled shake flasks (referred to as “Large Scale” (LS) culture herein). Cultures were collected by centrifugation in a swing-bucket centrifuge at 3000 rfc at 4° C. for 10 min. followed by removal of supernatant. The cell pellet was then resuspended in 1 mL of isotonic 375 mM sorbitol and re-pelleted in smaller 1.5 mL centrifuge tubes at 4000 rcf at 4° C. for 5 min. to remove residual salts. The sorbitol supernatant was then removed, and the cell pellet was flash-frozen by packing in dry ice before storage at −80° C. until use.

Purification of Capsids from Nannochloropsis oceanica

FIG. 17 provides a schematic of the AAV9 capsid purification protocol from IN21-transformed cells.

For each sample, about 150 mg of wet-biomass cell pellet was resuspended in 1200 μL of RT Algae Virus-like particle Isolation Buffer (AVIB) (500 mM NaCl, 100 mM Trisodium citrate pH 8.2, 1 mM EDTA, 0.3% vol./vol. NP-40 and 0.3% wt./vol sodium deoxycholate detergent supplemented with 1× Halt protease and phosphatase inhibitor (Thermofisher) and 1 mM DTT). 60 μL of AAV dilutant Buffer (150 mM NaCl, 20 mM sodium phosphate buffer pH 7.2, 0.01% Pluronic F-68) was added to each sample. For the positive control, 60 μL of AAV dilutant buffer contained 5e9 total AAV9 capsids produced in mammalian cells.

The suspended sample was loaded into 2 mL Lysing Matrix E (MP Biomedicals) tubes and disrupted in a Qiagen TissuelyserII. Four 1 min. cycles of 30 Hz of bead-beating with 1 min. rest times between each cycle were used to lyse the cells. The resulting emulsion was resolved by centrifuging at 3000 rcf for 5 min. at 20° C. followed by addition of 25 μL of cell wall enzyme master mix containing 0.4 mg/mL Cellulase R-10, 0.4 mg/mL Macerozyme R-10 (both from Goldbio), 5000 U/mL of Basemuncher nuclease (Abcam), and 200 mM MgCl₂ in 100 mM citrate buffer pH 3.8. The algae lysate was put on an end-over-end rotator at RT for 10 min. to degrade the cell walls. The lysate was then snap-frozen in crushed dry ice, quick thawed in a 25° C. water bath and bead-beat again in the Tissuelyser at 30 Hz for 1 min. before the freeze-thaw, and bead-beating were repeated.

The resulting frothy-emulsion was resolved by centrifuging at 3000 rcf for 5 min. at 20° C. 900 μL of supernatant was recovered into a new tube. The remaining pellet was re-extracted by adding 300 μL of AVIB and inverting the tube 10× before re-pelleting the cell debris by centrifuging at 3000 rcf for 5 minutes at 20° C. Additional re-extracted lysate (300 μL) was then pooled with the first extraction (1200 μL total) and 2.8 mL of molecular biology grade water was added to the extraction. Cell wall debris was removed from this diluted lysate by centrifugation at 3000 rcf for 15 min. at 20° C., providing the Starting Lysate Fraction shown in FIG. 15. The clarified supernatant (Starting Lysate Fraction in FIG. 15) (3.6 mL) was mixed with 1 mL of ethanol stabilized chloroform and vortexed for 1 min. The emulsion was resolved by centrifugation at 3000 rcf for 15 min. at 20° C. The de-pigmented and clarified aqueous layer, containing intact capsids, was then recovered (~3.4 mL) into a new tube. The clarified aqueous layer was then applied to 50 μL packed volume of POROS™ CaptureSelect™ AAV9 Affinity Resin beads that had previously been washed in 25 mM Trish pH 7,5, 0.01% PF-68 (Low Salt Wash Buffer).

The single-chain camelid VH (heavy chain variable region) antibodies conjugated to the POROS anti-AAV9 resin selectively and specifically recognize the five-fold symmetry peak of AAV9 (Mietzsch et al. (2020). Mol. Ther., Methods Clin. Dev. 19, p. 362-373). The AAV9 capsids were captured by placing the bead-lysate emulsion on an end-over-end mixer at 20 RPM for 30 min. at room temperature. The emulsion of beads was resolved by centrifuging at 500 rcf×5 min. at 20° C. The supernatant was removed and the beads washed with 1 mL each of Low Salt Wash Buffer, then 25 mM Tris pH 7.5, 300 mM NaCl, 0.01% PF-68 High Salt Wash Buffer, then, again, with Low Salt Wash Buffer with centrifugation at 500 rcf for 5 min. each to pellet the beads. The AAV9 particles were then eluted by addition of 300 μL of 100 mM Glycine pH 2.5, 0.01% PF-68 for 5 min. before recovery of the acidic glycine fraction and neutralization by addition of 37.5 μL of 1M Tris, pH 9.0, 1.5M NaCl.

### Dot Blot Detection of Intact AAV9 Capsids

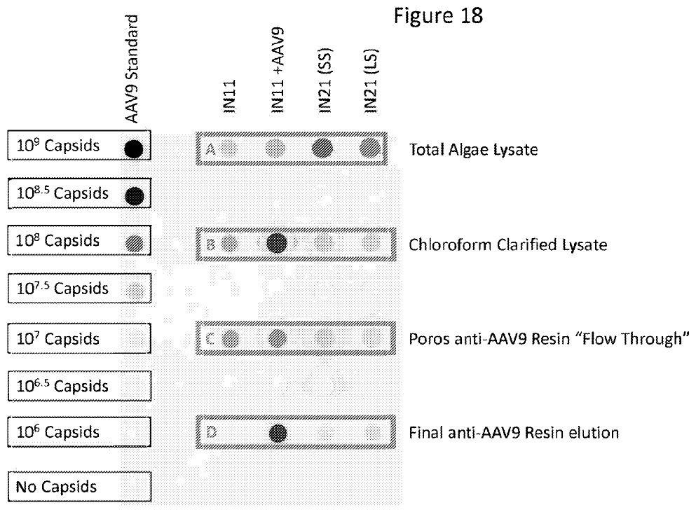

100 μL per well of (i) total algae lysate after bead-beating, (ii) chloroform clarified lysate, (iii) Poros anti-AAV9 bead-binding “flow through” (recovered supernatant after binding), or (iv) Poros anti-AAV9 bead elutant were loaded in to a 96-well vacuum-driven Bio-Dot array (Biorad) and bound to a 0.2 μm pore nitrocellulose membrane. The membrane was washed 3× with TBSMT and then probed with 1:20 dilution of anti-AAV9 ADK9 monoclonal antibody (Progen) overnight at 4° C. The membrane was washed again with TBSMT, and ADK9 binding detected using 1:1000 anti-mouse IgA alkaline phosphatase conjugated secondary antibody (Abcam) and pre-mixed BCIP/NBT developer (Thermofisher). FIG. 18 provides a dot-blot of algae lysates and purification intermediates.

In the dot-blot assay, an independent AAV9-specific monoclonal antibody (ADK9) recognized the affinity purified AAV9 particles. The ADK9 antibody specifically binds to a topologically distinct “valley” near the icosahedral 3-fold axes if of the intact AAV9 capsid (Emmanuel et al. (2022). Journal of Virology 96(3), e10251-21). These results demonstrate the isolation of intact putative AAV9 capsids from N. oceanica that are antigenically similar to AAV9 capsids produced in mammalian cell lines.

### Electron Microscopy

Poros anti-AAV9 bead elutant was negatively stained with uranyl nitrate as previously described and imaged using a scanning electron microscope.

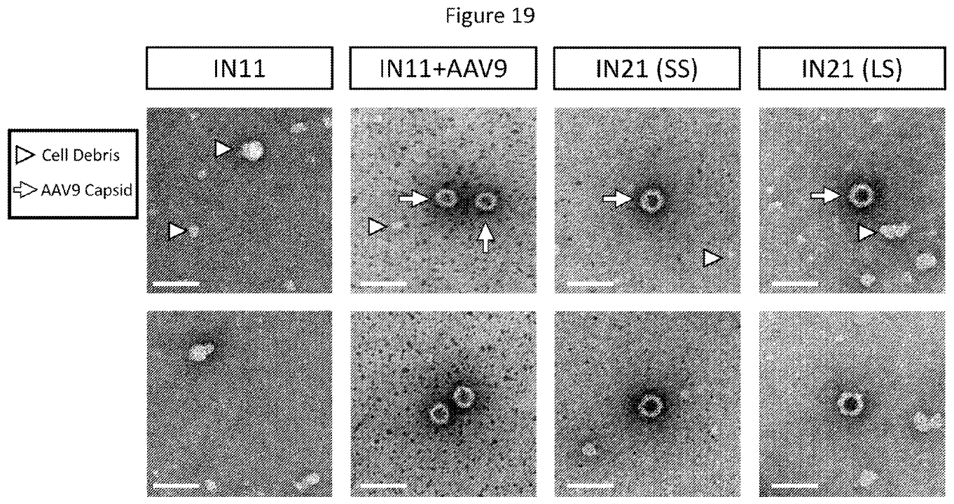

Uranyl nitrate negative staining of the putative purified AAV9 capsids demonstrates the presence of polyhedral structures matching the size and shape to AAV9 capsids produced in mammalian cells. See FIG. 19 for electron micrographs of identified capsids.

### SUMMARY

In summary, through biochemical, immunoassay, and microscopy based methods, the production and isolation of AAV9 capsids from an engineered strain of N. oceanica has been demonstrated.

### Example 3—Generation of a Rep-Protein Expression Vector in N. oceanica

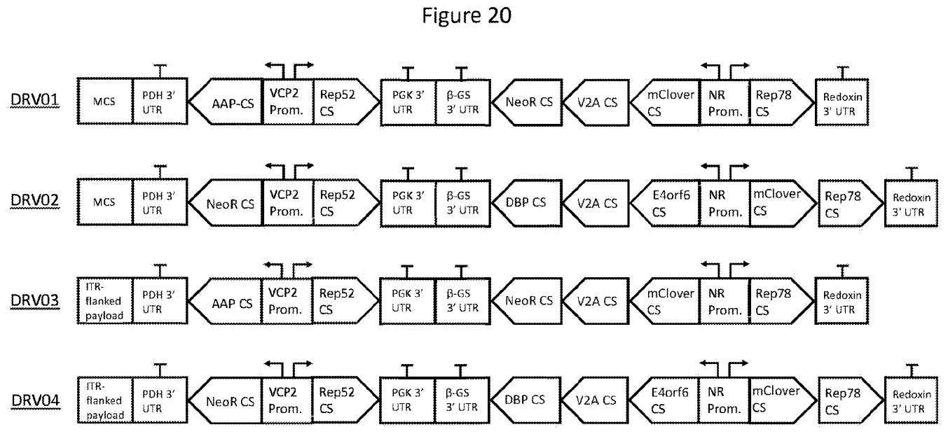

In this Example, it is reported that N. oceanica cells tolerated high levels of Rep52 expression but not Rep78. Specifically, genotypically correct clones for Ribi driven and Pol I driven Rep52 were recovered, but only the weaker Ribi promoter-driven Rep78 constructs were recovered after transformation. This led to the design of a dual-Rep78/Rep52 expression vector with an inducible promoter to drive Rep78 and a constitutively active promoter to drive Rep52. See FIG. 20. DRV01-DRV04 each uses the bi-directional N. oceanica nitrate-reductase promoter (SEQ ID NO:46) to drive a human-codon-reoptimized version of the AAV9 Rep78 coding sequence (SEQ ID NO:61) from one side of the promoter and a mClover coding sequence (SEQ ID NO: 65, coding for an enhanced Aequorea victoria Green Fluorescent Protein derivative) co-reporter from the other side of the promoter. The mClover reporter is linked by a viral 2A “skip” peptide coding sequence (SEQ ID NO: 13) to a neomycin resistance cassette (SEQ ID NO:51) for selection of transformants.

As shown in FIG. 20, DRV01-DRV04 each includes the following 3′ UTRs within the respective expression cassette. Redoxin 3′ UTR (SEQ ID NO:42), PGK 3′ UTR (SEQ ID NO: 43), PDH 3′ UTR (SEQ ID NO:44), B-GS 3′ UTR (SEQ ID NO:45).

The N. oceanica nitrate reductase promoter is active when the algae are grown on nitrate containing media with no other source of bioavailable nitrogen (e.g., no ammonium ions or urea) while the nitrate reductase promoter is off when cells are supplied with ammonium. Rep52 coding sequence in this construct (SEQ ID NO:60) was codon re-optimized to the Nicotiana benthamiana usage table to decrease homology to the Rep78 coding sequence (SEQ ID NO: 61) in order to minimize direct-repeats. The Rep52 coding sequence is driven by the constitutively active VCP2 promoter (SEQ ID NO:48) in addition to a codon re-optimized version of AAP9 containing a HA-epitope tag.

DRV01 can be transformed into IN21 cells by electroporation-mediated random integration and selection on G418-containing media to generate a capsid producing Rep52/78-expressing “general” production platform. Additionally, the DRV01 plasmid contains a multiple cloning site. This multiple cloning site was specifically designed with the intention of accepting sub-cloned sequences from common ITR-flanked gene therapy payload vectors (e.g., generating expression cassette DRV03). This ITR-payload and Rep52/78 expression vector can likewise be transformed into IN21 to produce a genome-packed capsid producing strain by use of only two large cassettes.

A variation of the dual-Rep52/78 expression vector was generated (DRV02, FIG. 20). Here, two adenoviral vector-derived gene coding sequences known to be directly involved in enhancing ITR-flanked DNA replication were included; DBP (SEQ ID NO:62) and E4orf6 (SEQ ID NO:63). These coding sequences are placed under the regulatory control of the nitrate reductase promoter while the neomycin resistance coding sequence (SEQ ID NO:51) is driven by the VCP2 promoter (SEQ ID NO:48) for transformant selection. Like DRV01, DRV02 can be integrated into a N. oceanica IN21 strain to produce a “general” Rep/Cap production platform. pDRV02 contains a multiple cloning site that can accept ITR-flanked payloads from commonly used gene therapy vectors (DRV04) and then transformed into IN21 to generate a specialized production platform. Further improvements and iterations can be made on vector pDRV02 where additional Adenoviral factors, such as the whole E1, E2A, E4, and VA genes can be added to further enhance AAV production. This can be accomplished through additional 2A-linked cDNA sequences downstream of the existing Adenoviral genes or the inclusion of additional algae promoter and 3′ UTR combinations.

When DRV01 or DRV02 are integrated into a capsid producing strain (e.g., IN21) an additional round of transformation can be used to introduce an ITR-flanked gene therapy cassette. In one example, an ITR-flanked gene therapy cassette can in integrated by random insertion (pUP01) followed by selection with blasticidin S which is an antibiotic that is orthogonal to both the neomycin resistance and hygromycin resistance selection cassettes used in other constructs.

Alternatively, CRISPR-mediated, homology-directed integration into a known locus can be used to deliver the ITR-flanked cassette (pUP02) followed by selection with blasticidin S. In the case of homology-directed integration one such integration locus is found between the 3′ UTRs of a MutS mismatch repair gene (transcript estExt_Genewise1.C_170242) and a HSP70 chaperon gene (transcript CE116060_6174). This site represents an open-chromatin locus ideal for locating an ITR-flanked payload because two constitutively transcribed “housekeeping” genes are located on each side of the locus but the site between the “facing-in” 3′UTRs is non-coding and unlikely regulatory. In a third iteration, the ITR-flanked payload can be electroporated into the Rep/Cap expressing algae as a circular plasmid containing the yeast CEN and ARS sequences that allow for autonomous replication of episomal DNA (plasmids) in N. oceanica. The episomes are maintained in the cells by continuous selection on blasticidin S.

The entire disclosure of each of the patent documents and scientific articles referred to herein is incorporated by reference for all purposes.

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

1. An expression cassette, comprising,
(i) a first Nannochloropsis-endogenous promoter comprising a first end and a second end, (ii) a first gene coding sequence operably linked to the first end of the first Nannochloropsis-endogenous promoter, (iii) a first Nannochloropsis-endogenous 3′ untranslated region (UTR) downstream of the first gene coding sequence and operably linked thereto, (iv) an antibiotic resistance gene coding sequence,
wherein the antibiotic resistance gene coding sequence is operably linked to the first Nannochloropsis-endogenous promoter or a second Nannochloropsis-endogenous promoter, and
wherein the first gene coding sequence is an adeno-associated virus (AAV) cap coding sequence.

2. The expression cassette of claim 1, wherein the AAV cap coding sequence is a VP1 coding sequence.

3. The expression cassette of claim 1, wherein the AAV cap coding sequence is a VP2 coding sequence.

4. The expression cassette of claim 1, wherein the AAV cap coding sequence is a VP3 coding sequence.

5. The expression cassette of claim 2, wherein the VP1 coding sequence is an AAV9 VP1 coding sequence.

6. The expression cassette of claim 3, wherein the VP2 coding sequence is an AAV9 VP2 coding sequence.

7. The expression cassette of claim 4, wherein the VP3 coding sequence is an AAV9 VP3 coding sequence.

8. The expression cassette of claim 5, wherein the AAV9 VP1 coding sequence comprises the nucleic acid sequence of SEQ ID NO:31.

9. The expression cassette of claim 6, wherein the AAV9 VP2 coding sequence comprises the nucleic acid sequence of SEQ ID NO:32 or 33.

10. The expression cassette of claim 7, wherein the AAV9 VP3 coding sequence comprises the nucleic acid sequence of SEQ ID NO:34 or 35.

11. The expression cassette of claim 1, wherein the AAV cap coding sequence is an AAV1, AAV2, AAV3, AAV4, AAV5, AAV6, AAV7, AAV8, AAV9, AAV10, AAVrh.74, AAVrh.8, AAVrh.10, AAV11, AAV12, AAV13 cap coding sequence, or a variant of one of the foregoing.

12. The expression cassette of claim 1, wherein the AAV cap coding sequence is an AAV9 cap gene coding sequence.

13. The expression cassette of claim 1, further comprising an assembly activating protein (AAP) coding sequence operably linked to the first Nannochloropsis-endogenous promoter, or operably linked to a second Nannochloropsis-endogenous promoter.

14. The expression cassette of claim 13, wherein the AAP coding sequence is an AAV9 AAP coding sequence.

15. The expression cassette of claim 13, wherein the AAP coding sequence comprises the nucleic acid sequence of SEQ ID NO: 57 or 76.

16. The expression cassette of any one of claims 13-15, wherein the AAP coding sequence is operably linked to the first Nannochloropsis-endogenous promoter.

17. The expression cassette of any one of claims 13-15, wherein the AAP coding sequence is operably linked to the second Nannochloropsis-endogenous promoter.

18. The expression cassette of any one of claims 13-17, wherein the AAP coding sequence is an AAV9 AAP sequence.

19. The expression cassette of any one of claims 1-18, wherein the antibiotic resistance coding sequence is a zeocin, neomycin, G418, kanamycin, rifampicin, benomyl, nystatin, spectinomycin, ampicillin, apramycin, hygromycin B or chloramphenicol resistance coding sequence.

20. The expression cassette of claim 19, wherein the antibiotic resistance coding sequence is a hygromycin B resistance coding sequence.

21. The expression cassette of claim 20, wherein the hygromycin B resistance coding sequence comprises the nucleic acid sequence of SEQ ID NO:52.

22. The expression cassette of claim 19, wherein the antibiotic resistance coding sequence is a neomycin resistance coding sequence.

23. The expression cassette of claim 20, wherein the neomycin resistance coding sequence comprises the nucleic acid sequence of SEQ ID NO:51.

24. The expression cassette of any one of claims 1-23, wherein the first Nannochloropsis-endogenous 3′ UTR comprises a lipid droplet surface protein (LDSP) 3′ UTR.

25. The expression cassette of claim 24, wherein the LDSP 3′ UTR comprises the nucleic acid sequence of SEQ ID NO:36.

26. The expression cassette of any one of claims 1-23, wherein the first Nannochloropsis-endogenous 3′ UTR is the VCP1 3′ UTR.

27. The expression cassette of any one of claims 1-23, wherein the first Nannochloropsis-endogenous 3′ UTR is the VCP2 3′ UTR.

28. The expression cassette of any one of claims 1-23, wherein the first Nannochloropsis-endogenous 3′ UTR is the heat shock protein 3′ UTR.

29. The expression cassette of any one of claims 1-23, wherein the first Nannochloropsis-endogenous 3′ UTR is the cellulose synthase (CS) 3′ UTR.

30. The expression cassette of any one of claims 1-29, wherein the first Nannochloropsis-endogenous promoter is a bidirectional promoter.

31. The expression cassette of claim 30, wherein the bidirectional promoter is the ribosomal subunit bidirectional (Ribi) promoter.

32. The expression cassette of claim 31, wherein the Ribi promoter comprises the nucleic acid sequence set forth in SEQ ID NO:1.

33. The expression cassette of claim 31, wherein the Ribi promoter comprises the nucleic acid sequence set forth in SEQ ID NO:2.

34. The expression cassette of claim 30, wherein the bidirectional promoter is a ribosomal subunit bidirectional promoter (Ribi), a nitrate reductase (NR) bidirectional promoter, or a violaxanthin chlorophyll a-binding protein (VCP) bidirectional promoter.

35. The expression cassette of claim 30, wherein the bidirectional promoter is a nitrate reductase (NR) bidirectional promoter.

36. The expression cassette of claim 30, wherein the bidirectional promoter is a violaxanthin chlorophyll a-binding protein (VCP) bidirectional promoter.

37. The expression cassette of any one of claims 1-26, wherein the first Nannochloropsis-endogenous promoter is a ubiquitin extension protein (UEP) promoter, a violaxanthin chlorophyll a-binding protein (VCP) promoter, a β-tubulin (β-tub) promoter, a lipid droplet surface protein (LDSP) promoter, an elongation factor (EF) promoter, a ribosomal Pol I promoter, or a ribosomal subunit bidirectional promoter (Ribi).

38. The expression cassette of claim 37, wherein the first Nannochloropsis-endogenous promoter is the violaxanthin chlorophyll a-binding protein (VCP) promoter.

39. The expression cassette of claim 38, wherein the VCP promoter is a VCP2 promoter comprising the nucleic acid sequence of SEQ ID NO:48.

40. The expression cassette of claim 38, wherein the VCP promoter is a VCP1 promoter.

41. The expression cassette of claim 40, wherein the VCP1 promoter comprises the nucleic acid sequence of SEQ ID NO: 47.

42. The expression cassette of claim 37, wherein the first Nannochloropsis-endogenous promoter is the ribosomal Pol I promoter.

43. The expression cassette of claim 42, wherein the ribosomal Pol I promoter comprises the nucleic acid sequence of SEQ ID NO:49.

44. The expression cassette of claim 37, wherein the first Nannochloropsis-endogenous promoter is the β-tubulin (β-tub) promoter.

45. The expression cassette of claim 37, wherein the promoter is an elongation factor (EF) promoter.

46. The expression cassette of claim 45, wherein the EF promoter comprises the nucleic acid sequence of SEQ ID NO:50.

47. The expression cassette of any one of claims 1-46, wherein the antibiotic resistance gene coding sequence is operably linked to a second Nannochloropsis-endogenous 3′ UTR.

48. The expression cassette of any one of claims 1-46, wherein the antibiotic resistance gene coding sequence is operably linked to the first Nannochloropsis-endogenous 3′ UTR.

49. The expression cassette of any one of claims 1-48, further comprising,
a first Nannochloropsis-endogenous 5′ UTR downstream of the first end of the first Nannochloropsis-endogenous promoter and upstream of the first gene coding sequence, wherein the first Nannochloropsis-endogenous 5′ UTR is operably linked to the first gene coding sequence.

50. The expression cassette of claim 49, wherein the first Nannochloropsis-endogenous 5′ UTR is derived from a different gene than the first Nannochloropsis-endogenous 3′ UTR.

51. The expression cassette of claim 49, wherein the first Nannochloropsis-endogenous 5′ UTR is derived from the same gene than the first Nannochloropsis-endogenous 3′ UTR.

52. The expression cassette of any one of claims 1-51, further comprising,
a 2A peptide coding sequence downstream of the first gene coding sequence, and a second gene coding sequence downstream of the 2A peptide coding sequence and upstream of the first Nannochloropsis-endogenous 3′ UTR, and operably linked thereto,
wherein the second gene coding sequence is operably linked to the first end of the first Nannochloropsis-endogenous promoter,
wherein the second gene coding sequence is the antibiotic resistance coding sequence, or
wherein the second gene coding sequence is an adeno-associated virus (AAV) cap coding sequence, or an AAV assembly activating protein (AAP) gene coding sequence, and
the second gene coding sequence is different from the first gene coding sequence.

53. The expression cassette of claim 52, wherein the 2A peptide encoded by the 2A peptide coding sequence comprises an amino acid sequence of SEQ ID NO:3, 4, 5, 6, 7, 8, 9 or 10.

54. The expression cassette of claim 52 or 53, wherein the second gene coding sequence is a second AAV cap coding sequence.

55. The expression cassette of claim 54, wherein the second AAV cap coding sequence is a VP1 coding sequence.

56. The expression cassette of claim 54, wherein the second AAV cap coding sequence is a VP2 coding sequence.

57. The expression cassette of claim 54, wherein the second AAV cap coding sequence is a VP3 coding sequence.

58. The expression cassette of claim 55, wherein the second AAV cap coding sequence is an AAV9 VP1 coding sequence.

59. The expression cassette of claim 56, wherein the second AAV cap coding sequence is an AAV9 VP2 coding sequence.

60. The expression cassette of claim 57, wherein the second AAV cap coding sequence is an AAV9 VP3 coding sequence.

61. The expression cassette of claim 58, wherein the second AAV cap coding sequence comprises the nucleic acid sequence of SEQ ID NO:31.

62. The expression cassette of claim 59, wherein the second AAV cap coding sequence comprises the nucleic acid sequence of SEQ ID NO:32 or 33.

63. The expression cassette of claim 60, wherein the second AAV cap coding sequence comprises the nucleic acid sequence of SEQ ID NO:34 or 35.

64. The expression cassette of claim 54, wherein the second AAV cap gene coding sequence is an AAV1, AAV2, AAV3, AAV4, AAV5, AAV6, AAV7, AAV8, AAV9, AAV10, AAVrh.74, AAVrh.8, AAVrh.10, AAV11, AAV12, AAV13 cap coding sequence or a variant of one of the foregoing.

65. The expression cassette of claim 54, wherein the second AAV cap coding sequence is an AAV9 cap gene coding sequence.

66. The expression cassette of claim 52 or 53, wherein the second gene coding sequence is an assembly activating protein (AAP) coding sequence.

67. The expression cassette of claim 66, wherein the second gene coding sequence is an AAV9 AAP coding sequence.

68. The expression cassette of claim 67, wherein the second gene coding sequence comprises the nucleic acid sequence of SEQ ID NO:57 or 76.

69. The expression cassette of claim 52 or 53, wherein the second gene coding sequence is the antibiotic resistance coding sequence.

70. The expression cassette of any one of claims 1-36 and 49-69, wherein the first Nannochloropsis-endogenous promoter is a bidirectional promoter.

71. The expression cassette of claim 70, further comprising,
a third gene coding sequence operably linked to the second end of the bidirectional promoter, and a second Nannochloropsis-endogenous 3′ UTR downstream of the third gene coding sequence and operably linked thereto, and
wherein the third gene coding sequence is the antibiotic resistance coding sequence, or,
wherein the third gene coding sequence is an adeno-associated virus (AAV) cap coding sequence or an AAV assembly activating protein (AAP) gene coding sequence, and
wherein, the third gene coding sequence is different from the first and second gene coding sequences.

72. The expression cassette of claim 71, wherein the third gene coding sequence is the antibiotic resistance coding sequence, and the second gene coding sequence is an adeno-associated virus (AAV) cap coding sequence or an AAV assembly activating protein (AAP) gene coding sequence.

73. The expression cassette of claim 71, wherein the third gene coding sequence is an AAV cap coding sequence.

74. The expression cassette of claim 73, wherein the third gene coding sequence is a VP1 coding sequence.

75. The expression cassette of claim 73, wherein the third gene coding sequence is a VP2 coding sequence.

76. The expression cassette of claim 73, wherein the third gene coding sequence is a VP3 coding sequence.

77. The expression cassette of claim 74, wherein the third gene coding sequence is an AAV9 VP1 coding sequence.

78. The expression cassette of claim 75, wherein the third gene coding sequence is an AAV9 VP2 coding sequence.

79. The expression cassette of claim 76, wherein the third gene coding sequence is an AAV9 VP3 coding sequence.

80. The expression cassette of claim 77, wherein the third gene coding sequence comprises the nucleic acid sequence of SEQ ID NO:31.

81. The expression cassette of claim 78, wherein the third gene coding sequence comprises the nucleic acid sequence of SEQ ID NO:32 or 33.

82. The expression cassette of claim 79, wherein the third gene coding sequence comprises the nucleic acid sequence of SEQ ID NO:34 or 35.

83. The expression cassette of claim 73, wherein the third gene coding sequence is an AAV1, AAV2, AAV3, AAV4, AAV5, AAV6, AAV7, AAV8, AAV9, AAV10, AAVrh.74, AAVrh.8, AAVrh.10, AAV11, AAV12, AAV13 cap coding sequence, or a variant of one of the cap coding sequence.

84. The expression cassette of claim 73, wherein the third gene coding sequence is an AAV9 cap gene coding sequence.

85. The expression cassette of claim 71, wherein the third gene coding sequence is an assembly activating protein (AAP) coding sequence.

86. The expression cassette of claim 85, wherein the third gene coding sequence is an AAV9 AAP coding sequence.

87. The expression cassette of claim 86, wherein the third gene coding sequence comprises the nucleic acid sequence of SEQ ID NO:57 or 76.

88. The expression cassette of any one of claims 71-87, further comprising a second Nannochloropsis-endogenous 5′ UTR downstream of the second end of the bidirectional promoter and upstream of the third gene coding sequence, wherein the second Nannochloropsis-endogenous 5′ UTR is operably linked to the third gene coding sequence and the second end of the bidirectional promoter.

89. The expression cassette of claim 88, wherein the second Nannochloropsis-endogenous 5′ UTR is derived from a different gene sequence than the second Nannochloropsis-endogenous 3′ UTR.

90. The expression cassette of claim 88, wherein the second Nannochloropsis-endogenous 5′ UTR is derived from the same gene sequence as the second Nannochloropsis-endogenous 3′ UTR.

91. The expression cassette of any one of claims 71-93, wherein the second 3′ UTR is the VCP1 3′ UTR, the VCP2 3′ UTR, the LDSP 3′ UTR, the heat shock protein 3′ UTR or the cellulose synthase (CS) 3′ UTR.

92. The expression cassette of any one of claims 71-91, further comprising,
a second 2A peptide coding sequence downstream of the third gene coding sequence, and a fourth gene coding sequence downstream of the second 2A peptide coding sequence and upstream of the second Nannochloropsis-endogenous 3′ UTR,
wherein the second 2A peptide coding sequence and the fourth gene coding sequence are each operably linked to the bidirectional promoter second end,
wherein the fourth gene coding sequence is the antibiotic resistance coding sequence, or,
wherein the fourth gene coding sequence is an adeno-associated virus (AAV) cap coding sequence or an AAV assembly activating protein (AAP) gene coding sequence, and the fourth gene coding sequence is different from the first, second and third gene coding sequences.

93. The expression cassette of claim 92, wherein the second 2A peptide coding sequence encodes a 2A peptide comprising an amino acid sequence set forth in SEQ ID NO:3, 4, 5, 6, 7, 8, 9 or 10.

94. The expression cassette of claim 92 or 93, wherein the fourth gene coding sequence is an AAV cap coding sequence.

95. The expression cassette of claim 94, wherein the fourth gene coding sequence is a VP1 coding sequence.

96. The expression cassette of claim 94, wherein the fourth gene coding sequence is a VP2 coding sequence.

97. The expression cassette of claim 94, wherein the fourth gene coding sequence is a VP3 coding sequence.

98. The expression cassette of claim 95, wherein the fourth gene coding sequence is an AAV9 VP1 coding sequence.

99. The expression cassette of claim 96, wherein the fourth gene coding sequence is an AAV9 VP2 protein coding sequence.

100. The expression cassette of claim 97, wherein the fourth gene coding sequence is an AAV9 VP3 coding sequence.

101. The expression cassette of claim 98, wherein the fourth gene coding sequence comprises the nucleic acid sequence of SEQ ID NO:31.

102. The expression cassette of claim 99, wherein the fourth gene coding sequence comprises the nucleic acid sequence of SEQ ID NO:32 or 33.

103. The expression cassette of claim 100, wherein the fourth gene coding sequence comprises the nucleic acid sequence of SEQ ID NO:34 or 35.

104. The expression cassette of claim 94, wherein the AAV cap coding sequence is an AAV1, AAV2, AAV3, AAV4, AAV5, AAV6, AAV7, AAV8, AAV9, AAV10, AAVrh.74, AAVrh.8, AAVrh.10, AAV11, AAV12, AAV13 cap gene coding sequence or a variant of one of the foregoing.

105. The expression cassette of claim 94, wherein the AAV cap gene coding sequence is an AAV9 cap gene coding sequence.

106. The expression cassette of claim 92 or 93, wherein the fourth gene coding sequence is an assembly activating protein (AAP) coding sequence.

107. The expression cassette of claim 106, wherein the fourth gene coding sequence is an AAV9 AAP coding sequence.

108. The expression cassette of claim 106, wherein the fourth gene coding sequence comprises the nucleic acid sequence of SEQ ID NO:57 or 76.

109. The expression cassette of any one of claims 1-108, further comprising a second Nannochloropsis-endogenous promoter operably linked to a fifth gene coding sequence, wherein a 3′ UTR is downstream of the fifth gene coding and operably linked thereto, wherein the fifth gene coding sequence is different from the first, second, third and fourth gene coding sequences.

110. The expression cassette of claim 109, wherein the promoter is a unidirectional promoter.

111. The expression cassette of claim 109 or 110, wherein the second promoter is the ribosomal Pol I promoter.

112. The expression cassette of claim 111, wherein the ribosomal Pol I promoter comprises the nucleic acid sequence of SEQ ID NO:49.

113. The expression cassette of claim 109 or 110, wherein the second promoter is the β-tubulin (β-tub) promoter.

114. The expression cassette of claim 109 or 110, wherein the second promoter is an elongation factor (EF) promoter.

115. The expression cassette of claim 114, wherein the elongation factor (EF) promoter is the EF 1 promoter.

116. The expression cassette of claim 115, wherein the EF 1 promoter comprises the nucleic acid sequence of SEQ ID NO:50.

117. The expression cassette of any one of claims 109-116, wherein the fifth gene coding sequence is an AAV cap gene coding sequence.

118. The expression cassette of claim 117, wherein the fifth gene coding sequence is a VP1 coding sequence.

119. The expression cassette of claim 117, wherein the fifth gene coding sequence is a VP2 coding sequence.

120. The expression cassette of claim 117, wherein the fifth gene coding sequence is a VP3 coding sequence.

121. The expression cassette of claim 118, wherein the fifth gene coding sequence is an AAV9 VP1 coding sequence.

122. The expression cassette of claim 119, wherein the fifth gene coding sequence is an AAV9 VP2 coding sequence.

123. The expression cassette of claim 120, wherein the fifth gene coding sequence is an AAV9 VP3 coding sequence.

124. The expression cassette of claim 121, wherein the AAV9 VP1 coding sequence comprises the nucleic acid sequence of SEQ ID NO:31.

125. The expression cassette of claim 122, wherein the AAV9 VP2 coding sequence comprises the nucleic acid sequence of SEQ ID NO:32 or 33.

126. The expression cassette of claim 123, wherein the AAV9 VP3 coding sequence comprises the nucleic acid sequence of SEQ ID NO:34 or 35.

127. The expression cassette of claim 117, wherein the fifth gene coding sequence is an AAV cap gene coding sequence selected from an AAV1, AAV2, AAV3, AAV4, AAV5, AAV6, AAV7, AAV8, AAV9, AAV10, AAVrh.74, AAVrh.8, AAVrh.10, AAV11, AAV12, and an AAV13 cap gene coding sequence, or a variant of one of the cap gene coding sequence.

128. The expression cassette of claim 117, wherein the fifth gene coding sequence is an AAV9 cap gene coding sequence.

129. The expression cassette of any one of claims 109-116, wherein the fifth gene coding sequence is an assembly activating protein (AAP) coding sequence.

130. The expression cassette of claim 129, wherein the fifth gene coding sequence is an AAV9 AAP coding sequence.

131. The expression cassette of claim 129, wherein the fifth gene coding sequence is an AAP coding sequence comprising the nucleic acid sequence of SEQ ID NO: 57 or 76.

132. The expression cassette of any one of claims 109-116, wherein the fifth gene coding sequence is the antibiotic resistance coding sequence.

133. The expression cassette of any one of claims 109-132, wherein the 3′ UTR downstream of the fifth coding sequence is an alpha-tubulin 3′ UTR.

134. The expression cassette of claim 133, wherein the alpha-tubulin 3′ UTR comprises the nucleic acid sequence of SEQ ID NO:37 or 38.

135. The expression cassette of any one of claims 109-134, wherein the 3′ UTR downstream of the fifth coding sequence is a dox9 3′ UTR.

136. The expression cassette of any one of claims 1-135, further comprising a third Nannochloropsis-endogenous promoter operably linked to a sixth nucleic acid coding sequence, wherein a third Nannochloropsis-endogenous 3′ UTR is downstream of the sixth gene coding and operably linked thereto, and the sixth gene coding sequence is different from the first, second, third, fourth and fifth gene coding sequences.

137. The expression cassette of claim 136, wherein the third promoter is a unidirectional promoter.

138. The expression cassette of claim 136, wherein the third promoter is the ribosomal Pol I promoter.

139. The expression cassette of claim 138, wherein the ribosomal Pol I promoter comprises the nucleic acid sequence of SEQ ID NO:49.

140. The expression cassette of claim 136, wherein the third promoter is the β-tubulin (β-tub) promoter.

141. The expression cassette of claim 136, wherein the third promoter is an elongation factor (EF) promoter.

142. The expression cassette of claim 141, wherein the elongation factor (EF) promoter is the EF 1 promoter.

143. The expression cassette of claim 142, wherein the EF 1 promoter comprises the nucleic acid sequence of SEQ ID NO:50.

144. The expression cassette of any one of claims 136-143, wherein the sixth gene coding sequence is an AAV cap coding sequence.

145. The expression cassette of claim 144, wherein the sixth gene coding sequence is a VP1 protein coding sequence.

146. The expression cassette of claim 144, wherein the sixth gene coding sequence is a VP2 coding sequence.

147. The expression cassette of claim 144, wherein the sixth gene coding sequence is a VP3 coding sequence.

148. The expression cassette of claim 145, wherein the sixth gene coding sequence is a AAV9 VP1 coding sequence.

149. The expression cassette of claim 146, wherein the sixth gene coding sequence is a AAV9 VP2 protein coding sequence.

150. The expression cassette of claim 147, wherein the sixth gene coding sequence is a AAV9 VP3 protein coding sequence.

151. The expression cassette of claim 148, wherein the sixth gene coding sequence is an AAV9 VP1 coding sequence comprising the nucleic acid sequence of SEQ ID NO:31.

152. The expression cassette of claim 149, wherein the sixth gene coding sequence is an AAV9 VP2 protein coding sequence comprising the nucleic acid sequence of SEQ ID NO:32 or 33.

153. The expression cassette of claim 150, wherein the sixth gene coding sequence is an AAV9 VP3 protein coding sequence comprising the nucleic acid sequence of SEQ ID NO:34 or 35.

154. The expression cassette of claim 144, wherein the sixth gene coding sequence is an AAV cap gene coding sequence selected from an AAV1, AAV2, AAV3, AAV4, AAV5, AAV6, AAV7, AAV8, AAV9, AAV10, AAVrh.74, AAVrh.8, AAVrh.10, AAV11, AAV12, and an AAV13 cap gene coding sequence, or a variant of one of the foregoing.

155. The expression cassette of claim 144, wherein the sixth gene coding sequence is an AAV9 cap coding sequence.

156. The expression cassette of any one of claims 136-143, wherein the sixth gene coding sequence is an assembly activating protein (AAP) coding sequence.

157. The expression cassette of claim 156, wherein the sixth gene coding sequence is an AAV9 AAP coding sequence.

158. The expression cassette of claim 157, wherein the sixth gene coding sequence is an AAP coding sequence comprising the nucleic acid sequence of SEQ ID NO:57 or 72.

159. The expression cassette of any one of claims 136-143, wherein the sixth gene coding sequence is the antibiotic resistance coding sequence.

160. The expression cassette of any one of claims 1-159, further comprising a 3′ flanking ribosomal DNA (rDNA) compatible homology arm and a 5′ flanking rDNA-compatible homology arm.

161. The expression cassette of any one of claims 1-160, wherein the Nannochloropsis species is Nannochloropsis australis, Nannochloropsis gaditana, Nannochloropsis granulata, Nannochloropsis limnetica, Nannochloropsis oceanica, Nannochloropsis oculate, or Nannochloropsis salina.

162. The expression cassette of any one of claims 1-160, wherein the Nannochloropsis species is Nannochloropsis oceanica.

163. The expression cassette of claim 1, wherein the first Nannochloropsis-endogenous promoter is the ribosomal subunit bidirectional promoter (Ribi), the first gene coding sequence is an AAV9 VP1, VP2 or VP3 coding sequence, and the 3′ UTR downstream of the first coding sequence comprises the lipid droplet surface protein (LDSP) 3′ UTR.

164. The expression cassette of claim 163, wherein the antibiotic resistance gene coding sequence is operably linked to the second end of the Ribi promoter, and wherein the expression cassette further comprises (i) a 2A peptide downstream of the antibiotic resistance gene coding sequence and operably linked thereto, (ii) an assembly activating protein (AAP) gene coding sequence downstream of 2A peptide and operably linked to the second end of the Ribi promoter, and (iii) a 3′ UTR downstream of the AAP gene coding sequence and operably linked thereto.

165. The expression cassette of claim 163 or 164, further comprising a second Nannochloropsis-endogenous promoter operably linked to an AAV VP2 coding sequence, and operably linked to a 3′ untranslated region (UTR) downstream of the AAV VP2 coding sequence, wherein the first gene coding sequence is an AAV VP1 coding sequence.

166. The expression cassette of claim 163 or 164, further comprising a second Nannochloropsis-endogenous promoter operably linked to an AAV VP3 coding sequence, and operably linked to a 3′ untranslated region (UTR) downstream of the AAV VP3 coding sequence, wherein the first gene coding sequence is an AAV VP1 coding sequence.

167. The expression cassette of claim 163 or 164, further comprising a second Nannochloropsis-endogenous promoter operably linked to an AAV VP1 coding sequence, and a operably linked to a 3′ untranslated region (UTR) downstream of the AAV VP1 coding sequence, wherein the first gene coding sequence is an AAV VP2 coding sequence.

168. The expression cassette of claim 163 or 164, further comprising a second Nannochloropsis-endogenous promoter operably linked to an AAV VP3 coding sequence, and a operably linked to a 3′ untranslated region (UTR) downstream of the AAV VP3 coding sequence, wherein the first gene coding sequence is an AAV VP2 coding sequence.

169. The expression cassette of claim 163 or 164, further comprising a second Nannochloropsis-endogenous promoter operably linked to an AAV VP1 coding sequence, and a operably linked to a 3′ untranslated region (UTR) downstream of the AAV VP1 coding sequence, wherein the first gene coding sequence is an AAV VP3 coding sequence.

170. The expression cassette of claim 163 or 164, further comprising a second Nannochloropsis-endogenous promoter operably linked to an AAV VP2 coding sequence, and a operably linked to a 3′ untranslated region (UTR) downstream of the AAV VP2 coding sequence, wherein the first gene coding sequence is an AAV VP3 coding sequence.

171. The expression cassette of any one claims 165-170, wherein the 3′ untranslated region (UTR) operably linked to the second Nannochloropsis-endogenous promoter is an alpha tubulin 3′ UTR.

172. The expression cassette of claim 165, further comprising a third Nannochloropsis-endogenous promoter operably linked to an AAV VP3 coding sequence.

173. The expression cassette of claim 166, further comprising a third Nannochloropsis-endogenous promoter operably linked to an AAV VP2 coding sequence.

174. The expression cassette of claim 167, further comprising a third Nannochloropsis-endogenous promoter operably linked to an AAV VP3 coding sequence.

175. The expression cassette of claim 168, further comprising a third promoter operably linked to an AAV VP1 coding sequence.

176. The expression cassette of claim 169, further comprising a third promoter operably linked to an AAV VP2 coding sequence.

177. The expression cassette of claim 170, further comprising a third promoter operably linked to an AAV VP1 coding sequence.

178. The expression cassette of any one of claims 164-177, wherein the second promoter is the ribosomal Pol I promoter.

179. The expression cassette of claim 178, wherein the ribosomal Pol I promoter comprises the nucleic acid sequence of SEQ ID NO:49.

180. The expression cassette of any one of claims 164-177, wherein the second promoter is the β-tubulin (β-tub) promoter.

181. The expression cassette of any one of claims 164-177, wherein the second promoter is an elongation factor (EF) promoter.

182. The expression cassette of claim 181, wherein the elongation factor (EF) promoter is the EF 1 promoter.

183. The expression cassette of claim 182, wherein the EF 1 promoter comprises the nucleic acid sequence of SEQ ID NO:50.

184. The expression cassette of any one of claims 172-183, wherein the third Nannochloropsis-endogenous promoter is the ribosomal Pol I promoter.

185. The expression cassette of claim 184, wherein the ribosomal Pol I promoter comprises the nucleic acid sequence of SEQ ID NO:49.

186. The expression cassette of any one of claims 172-183, wherein the third Nannochloropsis-endogenous promoter is the β-tubulin (β-tub) promoter.

187. The expression cassette of any one of claims 172-183, wherein the third Nannochloropsis-endogenous promoter is an elongation factor (EF) promoter.

188. The expression cassette of claim 187, wherein the elongation factor (EF) promoter is the EF 1 promoter.

189. The expression cassette of claim 188, wherein the EF 1 promoter comprises the nucleic acid sequence of SEQ ID NO:50.

190. An expression cassette, comprising,
(i) a constitutively active Nannochloropsis-endogenous bidirectional promoter comprising a first end and a second end,
(ii) a first gene coding sequence operably linked to the first end of the constitutively active Nannochloropsis-endogenous bidirectional promoter, wherein the first gene coding sequence is a Rep52 coding sequence,
(iii) a first Nannochloropsis-endogenous 3′ untranslated region (UTR) downstream of the first gene coding sequence and operably linked thereto,
(iv) a second gene coding sequence operably linked to the second end of the constitutively active Nannochloropsis-endogenous bidirectional promoter, wherein the second gene coding sequence is selected from an AAV assembly activating protein (AAP) gene coding sequence, and an antibiotic resistance gene coding sequence,
(v) a second Nannochloropsis-endogenous 3′ untranslated region (UTR) downstream of the second gene coding sequence and operably linked thereto,
(vi) an inducible Nannochloropsis-endogenous bidirectional promoter comprising a first end and a second end,
(vii) a third gene coding sequence operably linked to the first end of the inducible Nannochloropsis-endogenous bidirectional promoter, wherein the third gene coding sequence is a Rep78 coding sequence,
(viii) a third Nannochloropsis-endogenous 3′ untranslated region (UTR) downstream of the third coding sequence and operably linked thereto,
(ix) a fourth gene coding sequence operably linked to the second end of the inducible Nannochloropsis-endogenous bidirectional promoter, wherein the fourth gene coding sequence is selected from an adenoviral gene coding sequence and an antibiotic resistance gene coding sequence, and
(x) a fourth Nannochloropsis-endogenous 3′ untranslated region (UTR) downstream of the fourth coding sequence and operably linked thereto,
wherein the expression cassette comprises a single antibiotic resistance gene coding sequence.

191. The expression cassette of claim 190, wherein the first, second, third and fourth Nannochloropsis-endogenous 3′ UTR are each selected from the phosphoglycerate kinase (PGK), pyruvate dehydrogenase (PDH), 1,3-β-D-glucan synthase (β-GS) and the Redoxin 3′ UTR.

192. The expression cassette of claim 190 or 191, wherein the first Nannochloropsis-endogenous 3′ UTR is the phosphoglycerate kinase (PGK) 3′ UTR.

193. The expression cassette of claim 192, wherein the phosphoglycerate kinase (PGK) 3′ UTR comprises the nucleic acid sequence of SEQ ID NO:43.

194. The expression cassette of any one of claims 190-193, wherein the second Nannochloropsis-endogenous 3′ UTR is the pyruvate dehydrogenase (PDH) 3′ UTR.

195. The expression cassette of claim 194, wherein the pyruvate dehydrogenase (PDH) 3′ UTR comprises the nucleic acid sequence of SEQ ID NO:44.

196. The expression cassette of any one of claims 190-195, wherein the third Nannochloropsis-endogenous 3′ UTR is the redoxin 3′ UTR.

197. The expression cassette of claim 196, wherein the redoxin 3′ UTR comprises the nucleic acid sequence of SEQ ID NO:42.

198. The expression cassette of any one of claims 190-197, wherein the fourth Nannochloropsis-endogenous 3′ UTR is the 1,3-β-D-glucan synthase (β-GS) 3′ UTR.

199. The expression cassette of claim 198, wherein the B-GS 3′ UTR comprises the nucleic acid sequence of SEQ ID NO:45.

200. The expression cassette of any one of claims 190-199, wherein the fourth gene coding sequence is an adenovirus E4orf6 coding sequence.

201. The expression cassette of claim 200, wherein the adenovirus E4orf6 coding sequence comprises the nucleic acid sequence of SEQ ID NO:63.

202. The expression cassette of any one of claims 190-199, wherein the fourth gene coding sequence is an adenovirus DNA binding protein (DBP) coding sequence.

203. The expression cassette of claim 202, wherein the adenovirus DBP coding sequence comprises the nucleic acid sequence of SEQ ID NO:62.

204. The expression cassette of any one of claims 190-203, further comprising,
a 2A peptide coding sequence downstream of the third gene coding sequence, and a fifth gene coding sequence downstream of the 2A peptide coding sequence and upstream of the third Nannochloropsis-endogenous 3′ UTR,
wherein the fifth gene coding sequence is operably linked to the first end of the inducible Nannochloropsis-endogenous bidirectional promoter, and is selected from an adenoviral gene coding sequence and an antibiotic resistance gene coding sequence.

205. The expression cassette of any one of claims 190-204, further comprising,
a 2A peptide coding sequence downstream of the fourth gene coding sequence, and a sixth gene coding sequence downstream of the 2A peptide coding sequence and upstream of the fourth Nannochloropsis-endogenous 3′ UTR,
wherein the sixth gene coding sequence is operably linked to the second end of the inducible Nannochloropsis-endogenous bidirectional promoter, and is selected from an adenoviral gene coding sequence and an antibiotic resistance gene coding sequence.

206. The expression cassette of claim 204 or 205, wherein the 2A peptide coding sequence encodes a peptide comprising an amino acid sequence of SEQ ID NO:3, 4, 5, 6, 7, 8, 9 or 10.

207. The expression cassette of claim 205 or 206, wherein the sixth gene coding sequence is different from the fifth gene coding sequence and is an E4orf6 or DBP coding sequence.

208. The expression cassette of any one of claims 190-207, further comprising an adeno-associated virus (AAV) transgene comprising a promoter operably linked to a therapeutic gene coding sequence, wherein the promoter and therapeutic gene coding sequence are flanked by a 3′ AAV inverted terminal repeat (ITR) and a 5′ AAV ITR.

209. A Nannochloropsis packaging cell comprising an expression cassette of any one of claims 1-189.

210. A Nannochloropsis packaging cell comprising an expression cassette of any one of claims 190-208.

211. A Nannochloropsis packaging cell comprising an expression cassette of any one of claims 1-189 and an expression cassette of any one of claims 190-208.

212. A Nannochloropsis packaging cell comprising an expression cassette of any one of claims 1-189 and an expression cassette of any one of claims 190-207.

213. The Nannochloropsis packaging cell of claim 212, further comprising an adeno-associated virus (AAV) transgene comprising a promoter operably linked to a therapeutic gene coding sequence, wherein the promoter and therapeutic gene coding sequence are flanked by a 3′ AAV inverted terminal repeat (ITR) and a 5′ AAV ITR.

214. The Nannochloropsis packaging cell of any one of claims 210-211 and 213, wherein the 5′ AAV ITR is a 5′ AAV2 ITR.

215. The Nannochloropsis packaging cell of any one of claims 210-211 and 213-214, wherein the 3′ AAV ITR is a 3′ AAV2 ITR.

216. The Nannochloropsis packaging cell of any one of claims 209-215, wherein the Nannochloropsis packaging cell is a Nannochloropsis australis, Nannochloropsis gaditana, Nannochloropsis granulata, Nannochloropsis limnetica, Nannochloropsis oceanica, Nannochloropsis oculate, or a Nannochloropsis salina packaging cell.

217. The Nannochloropsis packaging cell of claim 216, wherein the Nannochloropsis packaging cell is a Nannochloropsis granulata packaging cell.

218. The Nannochloropsis packaging cell of claim 216, wherein the Nannochloropsis packaging cell is Nannochloropsis limnetica packaging cell.

219. The Nannochloropsis packaging cell of claim 216, wherein the Nannochloropsis packaging cell is Nannochloropsis oceanica packaging cell.

220. A Nannochloropsis microalgae that produces a recombinant adeno-associated virus (rAAV).

221. The Nannochloropsis microalgae of claim 220, comprising an expression cassette of any one of claims 1-189, an expression cassette of any one of claims 190-207 and a therapeutic transgene comprising a promoter operably linked to a therapeutic gene coding sequence, wherein the promoter and therapeutic gene coding sequence are flanked by a 3′ AAV inverted terminal repeat (ITR) and a 5′ AAV ITR.

222. The Nannochloropsis microalgae of claim 220, comprising an expression cassette of any one of claims 1-189, and an expression cassette of any one of claims 190-208.

223. The Nannochloropsis microalgae of any one of claims 220-222, wherein the Nannochloropsis microalgae is Nannochloropsis australis, Nannochloropsis gaditana, Nannochloropsis granulata, Nannochloropsis limnetica, Nannochloropsis oceanica, Nannochloropsis oculate, or Nannochloropsis salina.

224. The Nannochloropsis microalgae of claim 223, wherein the Nannochloropsis microalgae is Nannochloropsis granulata.

225. The Nannochloropsis microalgae of claim 223, wherein the Nannochloropsis microalgae is Nannochloropsis limnetica.

226. The Nannochloropsis microalgae of claim 223, wherein the Nannochloropsis microalgae is Nannochloropsis oceanica.

227. The Nannochloropsis microalgae of any one of claims 220-226, wherein the 5′ AAV ITR is a 5′ AAV2 ITR.

228. The Nannochloropsis microalgae of any one of claims 220-227, wherein the 3′ AAV ITR is a 3′ AAV2 ITR.

229. A method of producing a recombinant adeno-associated virus (rAAV), comprising, growing the Nannochloropsis packaging cell of any one of claims 209-219.

230. The method of claim 229, wherein the rAAV is a gene therapy vector or a vaccine vector.

231. The method of claim 229 or 230, further comprising purifying the recombinant virus.
